(19) United States
(12) Patent Application Publication
(10) Pub. No.: US 2010/0069388 A1
(43) Pub. Date: Mar. 18, 2010

(54) N-HYDROXYACRYLAMIDE COMPOUNDS

(75) Inventors: Yoshikazu Inoue, Osaka (JP); Yoshiteru Eikyu, Tokyo (JP); Fumiyuki Shirai, Tokyo (JP)

Correspondence Address: OBLON, SPIVAK, MCCLELLAND MAIER & NEUSTADT, L.L.P., 1940 DUKE STREET, ALEXANDRIA, VA, 22314, US

(73) Assignee: ASTELLAS PHARMA INC., TOKYO (JP)

(21) Appl. No.: 12/517,290

(22) PCT Filed: Dec. 14, 2007
(86) PCT No.: PCT/JP2007/074605

§ 371 Date: Jun. 2, 2009

Related U.S. Application Data
(60) Provisional application No. 60/870,229, filed on Dec. 15, 2006.

(51) Int. Cl. A61K 31/5377; C07D 211/72; C07D 241/02; C07D 413/12; A61K 31/44; A61K 31/4965; A61P 3/10

(52) U.S. Cl. 514/235.8; 546/312; 544/336; 544/120; 514/352; 514/255.06

(57) ABSTRACT

A compound having the following formula (I): wherein —R¹ is hydrogen, optionally substituted lower alkyl, cyclo(lower)alkyl, cyclo(higher)alkyl, optionally substituted aryl, optionally substituted heterocyclyl, or aryl-fused cyclo(lower)alkyl, R² is hydrogen or halogen, Z is CH or N, X is formula (II) R³ is lower alkyl which may be substituted with —OH or optionally substituted aryl, or lower alkanoyl, R⁴ is hydrogen or lower alkyl, Y is optionally substituted lower alkylene, or a salt thereof. The compound is useful as a histone deacetylase inhibitor.

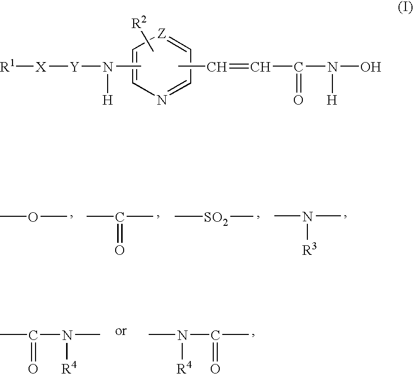

### TECHNICAL FIELD

The present invention relates to a compound useful as a medicament, and to a pharmaceutical composition comprising the same.

### BACKGROUND ART

Histone deacetylase (hereinafter also referred to as HDAC) is known to play an essential role in the transcriptional machinery for regulating gene expression, induce histone hyperacetylation and to affect the gene expression. Therefore, it is useful as a therapeutic or prophylactic agent for diseases caused by abnormal gene expression such as inflammatory disorders, diabetes, diabetic complications, homozygous thalassemia, fibrosis, cirrhosis, acute promyelocytic leukaemia (APL), organ transplant rejections, autoimmune diseases, protozoal infections, tumors, etc.

Many compounds which can inhibit the functions of the enzymes (HDAC inhibitors) has been studied extensively (see, e.g., WO2004/024160, US2004/087631, WO2004/063169, US2004/092558, WO2005/086898, WO2006/016680, WO2006/102760, WO2006/105979, WO2006/117548, WO2006/122319 etc).

For example, WO 01/38322 discloses an inhibitor of histone deacetylase represented by the following formula:

Cy-L¹-Ar—Y¹—C(O)—NH—Z

wherein
Cy is cycloalkyl, aryl, heteroaryl or heterocyclyl, each of which is optionally substituted;
L¹ is —(CH₂)ₘ—W— wherein m is an integer of 0 to 4, and W is selected from the group consisting of —C(O)NH—, —S(O)₂NH—, etc.;
Ar is optionally substituted arylene, which is optionally fused to an aryl, heteroaryl ring, etc.;
Y¹ is a chemical bond or a straight- or branched-chain saturated alkylene, wherein said alkylene is optionally substituted; and
Z is selected from the group consisting of anilinyl, pyridyl, thiadiazolyl and —O-M wherein M is H or a pharmaceutically acceptable cation.

WO 02/22577 discloses the following hydroxamate compound as a deacetylase inhibitor:

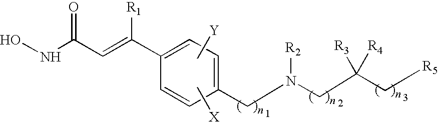

wherein
R₁ is H, halo or a straight chain C₁-C₆ alkyl;
R₂ is selected from H, C₁-C₁₀ alkyl, C₄-C₉ cycloalkyl, C₄-C₉ heterocycloalkyl, C₄-C₉ heterocycloalkylalkyl, cycloalkylalkyl, aryl, heteroaryl, etc.;
R₃ and R₄ are the same or different and independently H, C₁-C₆ alkyl, acyl or acylamino, or
R₃ and R₄ together with the carbon to which they are bound to represent C═O, C═S, etc., or
R₂together with the nitrogen to which it is bound and R₃ together with the carbon to which it is bound to form a C₄-C₉ heterocycloalkyl, a heteroaryl, a polyheteroaryl, anon-aromatic polyheterocycle, or a mixed aryl and non-aryl polyheterocycle ring;
R₅ is selected from H, C₁-C₆ alkyl, etc.;
n, n₁, n₂ and n₃ are the same or different and independently selected from 0-6, when n₁ is 1-6, each carbon atom can be optionally and independently substituted with R₃ and/or R₄;
X and Y are the same or different and independently selected from H, halo, C₁-C₄ alkyl, etc.;
or a pharmaceutically acceptable salt thereof.

### SUMMARY OF THE INVENTION

The present invention relates to a novel compound useful as a medicament, and to a pharmaceutical composition comprising the same.

More particularly, the present invention relates to a compound having a potent inhibitory effect on the activity of histone deacetylase.

The inventors of the present invention have also found that histone deacetylase inhibitors, such as a compound of the formula (I) (hereinafter compound (I)), have a potent immunosuppressive effect and potent antitumor effect. Therefore, a histone deacetylase inhibitors such as compound (I) is useful as an active ingredient for an immunosuppressant and an antitumor agent, and useful as an active ingredient for a therapeutic or prophylactic agent for diseases such as inflammatory disorders, diabetes, diabetic complications, homozygous thalassemia, fibrosis, cirrhosis, acute promyelocytic leukaemia (APL), organ transplant rejections, autoimmune diseases, protozoal infections, tumors, etc.

Accordingly, one object of the present invention is to provide a compound having biological activities for treating or preventing the diseases as stated above.

A further object of the present invention is to provide a pharmaceutical composition containing the compound (I) as an active ingredient.

A yet further object of the present invention is to provide use of the histone deacetylase inhibitors, such as compound (I), for treating and preventing the diseases as stated above.

A yet further object of the present invention is to provide a commercial package comprising the pharmaceutical composition containing the compound (I) and a written matter associated therewith, the written matter stating that the pharmaceutical composition may or should be used for treating or preventing the diseases as stated above.

Thus, the present invention provides a compound having the following formula (I):

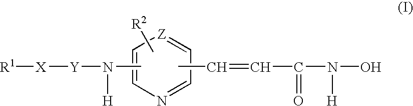

wherein
R¹ is hydrogen, optionally substituted lower alkyl, cyclo(lower)alkyl, cyclo(higher)alkyl, optionally substituted aryl, optionally substituted heterocyclyl, or aryl-fused cyclo(lower)alkyl,
R² is hydrogen or halogen,
Z is CH or N,
X is

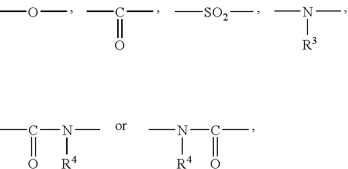

R³ is lower alkyl which may be substituted with —OH or optionally substituted aryl, or lower alkanoyl,
R⁴ is hydrogen or lower alkyl,
Y is optionally substituted lower alkylene,
or a salt thereof.

The above-mentioned compound or a salt thereof can be prepared by the process as illustrated in the following reaction scheme or by the methods disclosed in the Preparations and Examples.

In the above and subsequent descriptions of the present specification, suitable examples and illustration of the various definitions which the present invention intends to include within the scope thereof are explained in detail as follows.

The compound (I) of the present invention is obtained from compound (A), for example, according to the following process or methods disclosed in the Examples.

### Process 1

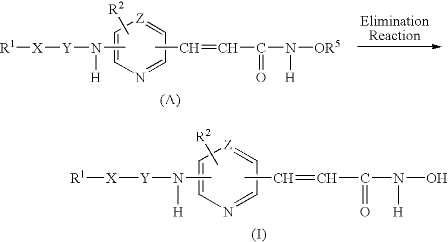

wherein R¹, R², X, Y and Z are each as defined above, and R⁵ is hydroxy protecting group.

### Process 1

The compound (I) is obtained by subjecting the compound (A) to the elimination reaction of hydroxy protecting group in the presence of an acid.

The acid includes such as hydrogen chloride solution (e.g. hydrogen chloride in solvent such as methanol, dioxane, ethyl acetate, diethyl ether, etc.), acetic acid, p-toluenesulfonic acid, boric acid, etc.

Optionally, one or more suitable solvent(s) for the deprotection is(are) used. Such solvent includes such as methanol, ethanol, ethyl acetate, dioxane, diethyl ether, acetic acid, etc.

The temperature of the reaction is not critical, and the reaction is usually carried out under cooling to heating.

The compound (I) may be a salt, which is also encompassed in the scope of the present invention. For example, when a basic group such as an amino group is present in a molecule, the salt is exemplified by an acid addition salt (e.g. salt with an inorganic acid such as hydrochloric acid, hydrobromic acid, sulfuric acid, etc., salt with an organic acid such as methanesulfonic acid, benzenesulfonic acid, 4-toluenesulfonic acid, camphorsulfonic acid (e.g., [(1S,4R)-7,7-dimethyl-2-oxobicyclo[2.2.1] hept-1-yl]methanesulfonic acid or an enantiomer thereof, etc.), fumaric acid, maleic acid, mandelic acid, citric acid, salicylic acid, malonic acid, glutaric acid, succinic acid, etc.), etc., and when an acidic group such as carboxyl group is present, the salt is exemplified by a basic salt (e.g. salt with a metal such as lithium, sodium, potassium, calcium, magnesium, aluminium, etc., a salt with amino acid such as lysine, etc.), etc.

In addition, solvates (e.g. hydrate, ethanolate, etc.), anhydrous forms and other polymorphic forms or pharmaceutically acceptable salts of the compound (I) are also encompassed in the scope of the present invention.

When the compound (I) has stereoisomers based on asymmetric carbon atom (s) or double bond (s), such as an optically active form, a geometric isomer and the like, such isomers and mixtures thereof are also encompassed in the scope of the present invention.

It is also to be noted that pharmaceutical acceptable prodrugs of the compound (I) are included within the scope of the present invention. Pharmaceutical acceptable prodrug means compound having functional groups which can be converted to —COOH, —NH₂, —OH etc. in physiological condition to form the compound (I) of the present invention.

In the above and subsequent descriptions of the present specification, suitable examples and illustration of the various definitions which the present invention intends to include within the scope thereof, are explained in detail as follows.

The term “halogen” means fluorine, chlorine, bromine and iodine.

The term “lower” used in the description is intended to mean 1 to 6 carbon atom(s) “C₁-C₆” unless otherwise indicated.

The term “higher” used in the description is intended to mean 7 to 11 carbon atom(s) unless otherwise indicated.

Suitable “one or more” may include the number of 1 to 6, preferably 1 to 3.

Suitable “lower alkyl” and “lower alkyl” moiety may include straight or branched alkyl having 1 to 6 carbon atom(s) such as methyl, ethyl, propyl, isopropyl, butyl, isobutyl, sec-butyl, tert-butyl, pentyl, tert-pentyl, neopentyl, hexyl, isohexyl, etc.

Suitable “cyclo(lower)alkyl” and “cyclo(lower)alkyl” moiety may include cycloalkyl having 3 to 6 carbon atoms such as cyclopropyl, cyclobutyl, cyclopentyl, cyclohexyl, etc.

Suitable “cyclo(higher)alkyl” and “cyclo(higher)alkyl” moiety may include cycloalkyl having 7 to 11 carbon atoms such as cycloheptyl, cyclooctyl, adamantyl, etc.

Suitable “lower alkylene” may include straight or branched alkylene having 1 to 6 carbon atom (s) such as methylene, ethylene, trimethylene, tetramethylene, pentamethylene, hexamethylene, methylmethylene, ethylmethylene, propylmethylene, isopropylmethylene, butylmethylene, isobutylmethylene, propylene, ethylethylene, 1,2-dimethylethylene, 1,1,2,2-tetramethylethylene, etc.

Suitable “aryl” or “ar” moiety may include C₆-C₁₆ aryl such as phenyl, naphthyl, anthryl, pyrenyl, phenanthryl, azulenyl, etc., and this “aryl” or “ar” moiety may be substituted with one or more substituent(s) selected from the group consisting of halogen and heterocyclyl(lower)alkyl.

Suitable “ar(lower)alkyl” may include phenyl(C₁-C₆)alkyl such as benzyl, phenethyl, phenylpropyl, phenylbutyl, phenylhexyl, etc., naphthyl(C₁-C₆)alkyl such as naphthylmethyl, naphthylethyl, naphthylpropyl, naphthylbutyl, naphthylpentyl, naphtylhexyl, etc.

Suitable “lower alkoxy” and “lower alkoxy” moiety may include straight or branched alkoxy having 1 to 6 carbon atom(s) such as methoxy, ethoxy, propoxy, isopropoxy, butoxy, isobutoxy, sec-butoxy, tert-butoxy, pentyloxy, tert-pentyloxy, neopentyloxy, hexyloxy, isohexyloxy, etc.

Suitable “ar(lower)alkoxy” may include phenyl (C₁-C₆) alkoxy such as benzyloxy, phenethyloxy, phenylpropoxy, phenylbutoxy, phenylhexyloxy, etc., naphthyl(C₁-C₆)alkoxy such as naphthylmethoxy, naphthylethoxy, naphthylpropoxy, naphthylbutoxy, naphthylpentyloxy, naphtylhexyloxy, etc.

Suitable “aryl-fused cyclo(lower)alkyl” and “aryl-fused cyclo(lower)alkyl” moiety may include aryl-fused cycloalkyl having 8 to 12 carbon atoms such as tetrahydronaphthyl, indanyl, benzocyclobutanyl, etc.

Suitable “lower alkanoyl” may include formyl and alkanoyl in which the alkyl portion is straight or branched alkyl having 1 to 5 carbon atom(s) such as acetyl, ethylcarbonyl, propylcarbonyl, isopropylcarbonyl, butylcarbonyl, isobutylcarbonyl, sec-butylcarbonyl, tert-butylcarbonyl, pentylcarbonyl, tert-pentylcarbonyl, neopentylcarbonyl, etc.

Suitable “carbamoyl optionally mono- or di-substituted with lower alkyl(s)” includes carbamoyl; N-(lower)alkylcarbamoyl in which the alkyl portion is alkyl having 1 to 6 carbon atom(s) such as N-methylcarbamoyl, N-ethylcarbamoyl, N-propylcarbamoyl, N-butylcarbamoyl, N-isobutylcarbamoyl, N-tert-butylcarbamoyl, N-pentylcarbamoyl, N-neopentylcarbamoyl, N-isopentylcarbamoyl, N-hexylcarbamoyl, etc.; N,N-di(lower)alkylcarbamoyl in which the alkyl portions are each alkyl having 1 to 6 carbon atom(s) such as N,N-dimethylcarbamoyl, N,N-diethylcarbamoyl, N,N-dipropylcarbamoyl, N,N-dibutylcarbamoyl, N,N-diisobutylcarbamoyl, N,N-di-tert-butylcarbamoyl, N,N-dipentylcarbamoyl, N,N-dineopentylcarbamoyl, N,N-diisopentylcarbamoyl, N,N-dihexylcarbamoyl, N-ethyl-N-methylcarbamoyl, N-methyl-N-propylcarbamoyl, N-butyl-N-methylcarbamoyl, N-methyl-N-isobutylcarbamoyl, etc. Each of these carbamoyl is optionally substituted by one or more suitable substituent(s).

Suitable “suitable substituent(s)” may include lower alkyl, aryl, cyclo(lower)alkyl, and the like.

Suitable example of “heteroaryl” and “heteroaryl” moiety may include unsaturated 3 to 8-membered (more preferably 5 or 6-membered) heteromonocyclic group containing 1 to 4 nitrogen atom(s), for example, pyrrolyl, pyrrolinyl, imidazolyl, pyrazolyl, pyridyl, dihydropyridyl, pyrimidyl, pyrazinyl, pyridazinyl, triazolyl (e.g., 4H-1,2,4-triazolyl, 1H-1,2,3-triazolyl, 2H-1,2,3-triazolyl, etc.), tetrazolyl (e. 1H-tetrazolyl, 2H-tetrazolyl, etc.), etc.;

Suitable example of “heterocyclyl” or “heterocyclyl” moiety may include

saturated 3 to 8-membered (more preferably 5 or 6-membered) heteromonocyclic group containing 1 to 4 nitrogen atom(s), for example, pyrrolidinyl, imidazolidinyl, piperidyl, piperazinyl, azetidinyl, etc.;

saturated 3 to 8-membered (more preferably 5 or 6-membered) heteromonocyclic group containing 1 or 2 oxygen atom(s) and 1 to 3 nitrogen atom(s), for example, morpholino, etc.;

and this “heterocyclyl” or “heterocyclyl” moiety may be substituted with one or more lower alkyl.

Suitable “hydroxy protecting group” is as follows:
lower alkyl (e.g., methyl, ethyl, propyl, isopropyl, butyl, isobutyl, t-butyl, pentyl, hexyl, etc.), preferably methyl;
lower alkoxy(lower)alkyl (e.g. methoxymethyl, etc.);
lower alkoxy(lower)alkoxy(lower)alkyl (e.g. 2-methoxyethoxymethyl, etc.);
ar(lower)alkyl in which the aryl portion is optionally substituted with one or more suitable substituent(s) (e.g. benzyl (Bn), p-methoxybenzyl, m,p-dimethoxybenzyl, etc.), preferably benzyl;
ar(lower)alkoxy(lower)alkyl in which the aryl portion is optionally substituted with one or more suitable substituent(s) (e.g. benzyloxymethyl, p-methoxybenzyloxymethyl, etc.);
(lower)alkylthio(lower)alkyl (e.g. methylthiomethyl, ethylthiomethyl, propylthiomethyl, isopropylthiomethyl, butylthiomethyl, isobutylthiomethyl, hexylthiomethyl, etc.), etc., preferably methylthiomethyl;
trisubstituted silyl such as tri(lower)alkylsilyl (e.g. trimethylsilyl, triethylsilyl, tributylsilyl, tert-butyldimethylsilyl, tri-tert-butylsilyl, etc.), lower alkyldiarylsilyl (e.g. methyldiphenylsilyl, ethyldiphenylsilyl, propyldiphenylsilyl, tert-butyldiphenylsilyl (TBDPS), etc.), etc., preferably tert-butyldimethylsilyl (TBDMS) and tert-butyldiphenylsilyl;
heterocyclic group (e.g. tetrahydropyranyl, etc.);
acyl as described below [e.g. aliphatic acyl such as lower alkanoyl (e.g. acetyl, propanoyl, pivaloyl, etc.); aromatic acyl (e.g. benzoyl (Bz), toluoyl, naphthoyl, fluorenylcarbonyl, etc.);
lower alkoxy-carbonyl (e.g. methoxycarbonyl, ethoxycarbonyl, propoxycarbonyl, isopropoxycarbonyl, butoxycarbonyl, isobutoxycarbonyl, t-butoxycarbonyl, pentyloxycarbonyl, hexyloxycarbonyl, etc.), etc.;
ar(lower)alkoxycarbonyl in which the aryl portion is optionally substituted with one or more suitable substituent(s) (e.g. benzyloxycarbonyl, bromobenzyloxycarbonyl, etc.);
lower alkylsulfonyl (e.g. methylsulfonyl, ethylsulfonyl, etc.);
lower alkoxysulfonyl (e.g. methoxysulfonyl, ethoxysulfonyl, etc.);
ar(lower)alkanoyl (e.g. phenylacetyl, phenylpropanoyl, phenylbutanoyl, phenylisobutanoyl, phenylpentanoyl, phenylhexanoyl, naphthylacetyl, naphthylpropanoyl, naphthylbutanoyl, naphthylisobutanoyl, naphthylpentanoyl, naphthylhexanoyl, etc.);
ar(lower)alkenoyl such as ar(C₃-C₆)alkenoyl (e.g. phenylpropenoyl, phenylbutenoyl, phenylmethacryloyl, phenylpentenoyl, phenylhexenoyl, naphthylpropenoyl, naphthylbutenoyl, naphthylmethacryloyl, naphthylpentenoyl, naphthylhexenoyl, etc.), etc.];
lower alkenyl (e.g. vinyl, allyl, etc.); etc.

The preferable hydroxy protecting group for the present invention is, for example, tetrahydropyranyl, trimethylsilyl, t-butyldimethylsilyl, etc.

The preferred embodiment of the present invention is shown as follow.

The compound having the formula (I), wherein

(1) a compound of the following formula (I′)

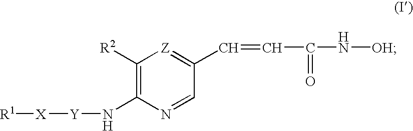

(2) R¹ is hydrogen, lower alkyl, cyclo(lower)alkyl(lower) alkyl, cyclo(higher)alkyl(lower)alkyl, optionally substituted ar(lower)alkyl, heteroaryl(lower)alkyl, cyclo(lower)alkyl, cyclo(higher)alkyl, optionally substituted aryl, lower alkyl heterocyclyl, aryl-fused cyclo (lower) alkyl and preferably, R¹ is cyclo(lower)alkyl(lower)alkyl, ar(lower)alkyl which may be substituted with halogen, cyclo(lower)alkyl, cyclo(higher)alkyl, or aryl which may be substituted with halogen, and more preferably, R¹ is cyclohexylmethyl, benzyl, chlorobenzyl, cyclopentyl, cyclohexyl, cycloheptyl, adamantyl, phenyl or chlorophenyl.;

(3) R² is hydrogen or halogen, and Z is CH or N, and preferably R² is hydrogen and Z is N, or R² is halogen and Z is CH, and more preferably, R² is hydrogen and Z is N, or R² is fluorine or chlorine and Z is CH.;

(4) X is

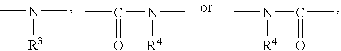

in which R³ is preferably lower alkyl which may be substituted with —OH or aryl substituted with halogen, or lower alkanoyl, and more preferably, R³ is lower alkyl or lower alkanoyl, and more preferably, R³ is methyl or acetyl, and most preferably, R³ is methyl, and R⁴ is hydrogen or lower alkyl, and more preferably, R⁴ is hydrogen or methyl, and most preferably, R⁴ is hydrogen.

(5) Y is lower alkylene which may be substituted with hydroxy, aryl, aryl(lower)alkoxy, or carbamoyl optionally mono- or di-substituted with lower alkyl(s), and preferably Y is lower alkylene, and more preferably, Y is ethylene, methylmetylene, ethylmethylene, isopropylmethylene, propylene or isobutylmethylene.;

(6) a compound that combined two or more of above-mentioned (1)-(5).

(7) a compound of above-mentioned (1) wherein
R¹ is hydrogen, lower alkyl, cyclo(lower)alkyl(lower)alkyl, cyclo(higher)alkyl(lower)alkyl, optionally substituted ar(lower)alkyl, heteroaryl(lower)alkyl, cyclo(lower)alkyl, cyclo(higher)alkyl, optionally substituted aryl, lower alkyl heterocyclyl, aryl-fused cyclo(lower)alkyl,
R² is hydrogen or halogen,
Z is CH or N,
X is

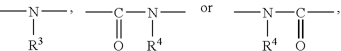

R³ is lower alkyl which may be substituted with —OH or aryl substituted with halogen, or lower alkanoyl,
R⁴ is hydrogen or lower alkyl,
Y is lower alkylene which may be substituted with hydroxy, aryl, aryl(lower)alkoxy, or carbamoyl optionally mono- or di-substituted with lower alkyl(s).

(8) a compound of above-mentioned (7) wherein
R¹ is cyclo(lower)alkyl(lower)alkyl, ar(lower)alkyl which may be substituted with halogen, cyclo(lower)alkyl, cyclo(higher)alkyl, or aryl which may be substituted with halogen,
R² is hydrogen and Z is N, or R² is halogen and Z is CH,
X is

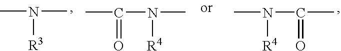

R³ is lower alkyl or lower alkanoyl,
R⁴ is hydrogen or lower alkyl,
Y is lower alkylene.

(9) a compound of above-mentioned (8) wherein
R¹ is cyclohexylmethyl, benzyl, chlorobenzyl, cyclopentyl, cyclohexyl, cycloheptyl, adamantyl, phenyl or chlorophenyl,
R² is hydrogen and Z is N, or R² is fluorine or chlorine and Z is CH,
X is

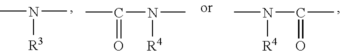

R³ is methyl or acetyl,
R⁴ is hydrogen or methyl,
Y is ethylene, methylmetylene, ethylmethylene, isopropylmethylene, propylene or isobutylmethylene.

### Test Method

In order to show the usefulness of the compound (I) of the invention, the pharmacological test result of the representative compound of the present invention is shown in the following.

### Test 1: Determination of Histone Deacetylase Inhibitor Activity

The partial purification of human histone deacetylase, the preparation of [³H] acetyl histones, and the assay for histone deacetylase activity were performed basically according to the method as proposed by Yoshida et al. as follows.

### Partial Purification of Human Histone Deacetylase

The human histone deacetylase was partially purified from human T cell leukemia Jurkat cells. Jurkat cells (5×10⁸ cells) were suspended in 40 mL of the HDA buffer consisting of 15 mM potassium phosphate, pH 7.5, 5% glycerol and 0.2 mM EDTA. After homogenization, nuclei were collected by centrifugation (35,000×g, 10 min) and homogenized in 20 mL of the same buffer supplemented with 1 M (NH₄)₂SO₄. The viscous homogenate was sonicated and clarified by centrifugation (35,000×g, 10 min), and the deacetylase was precipitated by raising the concentration of (NH₄)₂SO₄ to 3.5 M. The precipitated protein was dissolved in 10 mL of the HDA buffer and dialyzed against 4 liters of the same buffer. The dialyzate was then loaded onto a DEAE-cellulose (Whatman DE52) column (25×85 mm) equilibrated with the same buffer and eluted with 300 mL of a linear gradient (0-0.6 M) of NaCl. A single peak of histone deacetylase activity appeared between 0.3 and 0.4 M NaCl.

### Preparation of [³H] Acetyl Histone

To obtain [³H] acetyl-labeled histone as the substrate for the histone deacetylase assay, 1×10⁸ cells of Jurkat in 20 mL of RPMI-1640 medium (supplemented with 10% FBS, penicillin (50 units/mL) and streptomycin (50 μg/mL)) were incubated with 300 MBq [³H] sodium acetate in the presence of 5 mM sodium butyrate for 30 minutes in 5% CO₂-95% air atmosphere at 37° C. in a 75 cm² flask, harvested into a centrifuge tube (50 mL), collected by centrifugation at 1000 rpm for 10 minutes, and washed once with phosphate-buffered saline. The washed cells were suspended in 15 mL of ice-cold lysis buffer (10 mM Tris-HCl, 50 mM sodium bisulfite, 1% Triton X-100, 10 mM MgCl₂, 8.6% sucrose, pH 6.5). After Dounce homogenization (30 stroke), the nuclei were collected by centrifugation at 1000 rpm for 10 minutes, washed 3 times with 15 mL of the lysis buffer, and once with 15 mL of ice-cooled washing buffer (10 mM Tris-HCl, 13 mM EDTA, pH 7.4) successively. The pellet was suspended in 6 mL of ice-cooled water using a mixer, and 68 μl of H₂SO₄ was added to the suspension to give a concentration of 0.4 N. After incubation at 4° C. for 1 hour, the suspension was centrifuged for 5 minutes at 15,000 rpm, and the supernatant was taken and mixed with 60 mL of acetone. After overnight incubation at −20° C., the coagulated material was collected by microcentrifugation, air-dried, and stored at −80° C.

### Assay for Histone Deacetylase Activity

For the standard assay, 10 μl of [³H] acetyl-labeled histones were added to 90 μl of the enzyme fraction, and the mixture was incubated at 25° C. for 30 minutes. The reaction was stopped by addition of 10 μl of HCl aq. The released [³H] acetic acid was extracted with 1 mL of ethyl acetate, and 0.9 mL of the solvent layer was taken into 10 mL of toluene scintillation solution for determination of radioactivity.

### Test 2: Determination of T-Cell Growth Inhibitor Activity

The T lymphocyte blastogenesis test was performed in microtiter plates with each well containing 1.5×10⁵ splenic cells of Lewis rats in 0.1 mL RPMI-1640 medium supplemented with 10% fetal bovine serum (FBS), 50 mM 2-mercaptoethanol, penicilln (100 units/mL) and streptomycin (100 μg/mL), to which Concanavalin A (1 μg/mL) was added. The cells were incubated at 37° C. in a humidified atmosphere of 5% CO₂ for 72 hours. After the culture period, suppressive activities of the test compounds in T lymphocyte blastogenesis were quantified by AlamarBlue (trademark) Assay. The test samples were dissolved in DMSO and further diluted with RPMI-1640 medium and added to the culture. The activities of the test compounds were expressed as IC₅₀.

The results of those tests are shown in the Table 1.

TABLE 1
HDAC inhibitory activity and T-cell growth inhibitory
activity of the compound of the present invention

| | | Test 2: |
|---|---|---|
| | Test 1: | T-cell |
| | HDAC | growth |
| | inhibitory | inhibitory |
| | activity | activity |
| Examples | IC₅₀ (nM) | IC₅₀ (nM) |
| Example 3 | 1.7 | 18 |
| Example 10 | 6.8 | 17 |
| Example 11 | 8.8 | 6.2 |
| Example 23 | 6.0 | 21 |
| Example 36 | 4.0 | 1.5 |
| Example 39 | 0.78 | 0.23 |
| Example 49 | 25 | 17 |
| Example 57 | 9.1 | 4.7 |
| Example 66 | 3.9 | 21 |
| Example 86 | 8.2 | 28 |
| Example 87 | 2.3 | 3.2 |
| Example 88 | 2.7 | 1.5 |
| Example 91 | 1.5 | 2.6 |

An Ames examination is negative, and the object compounds are expected to be without decrease of a blood platelet/neutrophile, without decrease of blood pressure and without increase of heart rate at a dose of the efficacy of them.

The pharmaceutical composition of the present invention comprising histone deacetylase inhibitor such as the compound (I) is useful as,a therapeutic or prophylactic agent for diseases caused by abnormal gene expression, such as inflammatory disorders, diabetes, diabetic complications, homozygous thalassemia, fibrosis, cirrhosis, acute promyelocytic leukaemia (APL), protozoal infection, etc. Furthermore, it is useful as an antitumor agent or immunosuppressant, which prevents an organ transplant rejection and autoimmune diseases as exemplified below:
rejection reactions by transplantation of organs or tissues such as the heart, kidney, liver, bone marrow, skin, cornea, lung, pancreas, small intestine, limb, muscle, nerve, intervertebral disc, trachea, myoblast, cartilage, etc.;
graft-versus-host reactions following bone marrow transplantation;
autoimmune diseases such as rheumatoid arthritis, systemic lupus erythematosus, Hashimoto's thyroiditis, multiple sclerosis, myasthenia gravis, type I diabetes, etc.; and
infections caused by pathogenic microorganisms (e.g. Aspergillus fumigatus, Fusarium oxysporum, Trichophyton asteroides, etc.).

Furthermore, pharmaceutical preparations of the histone deacetylase inhibitor, such as the compound (I), are useful for the therapy or prophylaxis of the following diseases.

Inflammatory or hyperproliferative skin diseases or cutaneous manifestations of immunologically-mediated diseases (e.g. psoriasis, atopic dermatitis, contact dermatitis, eczematoid dermatitis, seborrheic dermatitis, lichen planus, pemphigus, bullous pemphigoid, epidermolysis bullosa, urticaria, angioedema, vasculitides, erythema, dermal eosinophilia, lupus erythematosus, acne, alopecia areata, etc.);.
autoimmune diseases of the eye (e.g. keratoconjunctivitis, vernal conjunctivitis, uveitis associated with Behcet's disease, keratitis, herpetic keratitis, conical keratitis, corneal epithelial dystrophy, keratoleukoma, ocular premphigus, Mooren's ulcer, scleritis, Grave's ophthalmopathy, Vogt-Koyanagi-Harada syndrome, keratoconjunctivitis sicca (dry eye), phlyctenule, iridocyclitis, sarcoidosis, endocrine ophthalmopathy, etc.);
reversible obstructive airways diseases [asthma (e.g. bronchial asthma, allergic asthma, intrinsic asthma, extrinsic asthma, dust asthma, etc.), particularly chronic or inveterate asthma (e.g. late asthma, airway hyper-responsiveness, etc.), bronchitis, etc.];
mucosal or vascular inflammations (e.g. gastric ulcer, ischemic or thrombotic vascular injury, ischemic bowel diseases, enteritis, necrotizing enterocolitis, intestinal damages associated with thermal burns, leukotriene B4-mediated diseases, etc.);
intestinal inflammations/allergies (e.g. coeliac diseases, proctitis, eosinophilic gastroenteritis, mastocytosis, Crohn's disease, ulcerative colitis, etc.);
food-related allergic diseases with symptomatic manifestation remote from the gastrointestinal tract (e.g. migraine, rhinitis, eczema, etc.);
renal diseases (e.g. intestitial nephritis, Goodpasture's syndrome, hemolytic uremic syndrome, diabetic nephropathy, etc.);
nervous diseases (e.g. multiple myositis, Guillain-Barre syndrome, Meniere's disease, multiple neuritis, solitary neuritis, cerebral infarction, Alzheimer's disease, Parkinson's disease, amyotrophic lateral sclerosis (ALS), radiculopathy, etc.);
cerebral ischemic diseases (e.g., head injury, hemorrhage in brain (e.g., subarachnoid hemorrhage, intracerebral hemorrhage, etc.), cerebral thrombosis, cerebral embolism, cardiac arrest, stroke, transient ischemic attack (TIA), hypertensive encephalopathy, etc.);
endocrine diseases (e.g. hyperthyroidism, Basedow's disease, etc.);
hematic diseases (e.g. pure red cell aplasia, aplastic anemia, hypoplastic anemia, idiopathic thrombocytopenic purpura, autoimmune hemolytic anemia, agranulocytosis, pernicious anemia, megaloblastic anemia, anerythroplasia, etc.);
bone diseases (e.g. osteoporosis, etc.);
respiratory diseases (e.g. sarcoidosis, pulmonary fibrosis, idiopathic interstitial pneumonia, etc.);
skin diseases (e.g. dermatomyositis, leukoderma vulgaris, ichthyosis vulgaris, photosensitivity, cutaneous T-cell lymphoma, etc.);
circulatory diseases (e.g. arteriosclerosis, atherosclerosis, aortitis syndrome, polyarteritis nodosa, myocardosis, etc.);
collagen diseases (e.g. scleroderma, Wegener's granuloma, Sjögren's syndrome, etc.);
adiposis;
eosinophilic fasciitis;
periodontal diseases (e.g. damage to gingiva, periodontium, alveolar bone or substantia ossea dentis, etc.);
nephrotic syndrome (e.g. glomerulonephritis, etc.);
male pattern alopecia, alopecia senile;
muscular dystrophy;
pyoderma and Sezary syndrome;
chromosome abnormality-associated diseases (e.g. Down's syndrome, etc.);
Addison's disease;
active oxygen-mediated diseases (e.g. organ injury [e.g. ischemic circulation disorders of organs (e.g. heart, liver, kidney, digestive tract, etc.) associated with preservation, transplantation, ischemic diseases (e.g. thrombosis, cardial infarction, etc.), etc.];
intestinal diseases (e.g. endotoxin shock, pseudomembranous colitis, drug- or radiation-induced colitis, etc.);
renal diseases (e.g. ischemic acute renal insufficiency, chronic renal failure, etc.);
pulmonary diseases (e.g. toxicosis caused by pulmonary oxygen or drugs (e.g. paracort, bleomycin, etc.), lung cancer, pulmonary emphysema, etc.);
ocular diseases (e.g. cataracta, iron-storage disease (siderosis bulbi), retinitis, pigmentosa, senile plaques, vitreous scarring, corneal alkali burn, etc.); dermatitis (e.g. erythema multiforme, linear immunoglobulin A bullous dermatitis, cement dermatitis, etc.); and
other diseases (e.g. gingivitis, periodontitis, sepsis, pancreatitis, diseases caused by environmental pollution (e.g. air pollution, etc.), aging, carcinogen, metastasis of carcinoma, hypobaropathy, etc.));
diseases caused by histamine release or leukotriene C4 release; restenosis of coronary artery following angioplasty and prevention of postsurgical adhesions;
autoimmune diseases and inflammatory conditions (e.g., primary mucosal edema, autoimmune atrophic gastritis, premature menopause, male sterility, juvenile diabetes mellitus, pemphigus vulgaris, pemphigoid, sympathetic ophthalmitis, lens-induced uveitis, idiopathic leukopenia, active chronic hepatitis, idiopathic cirrhosis, discoid lupus erythematosus, autoimmune orchitis, arthritis (e.g. arthritis deformans, etc.), polychondritis, etc.);
Human Immunodeficiency Virus (HIV) infection, AIDS;
allergic conjunctivitis;
hypertrophic cicatrix, keloid due to trauma, burn or surgery, vascular intimal hyperplasia, etc.

Furthermore, as an antiproliferative agent, HDAC inhibitor may have potential in the treatment of coronary artery disease, particularly in preventing restenosis in patients undergoing percutaneous transluminal coronary angiography (PTCA).

Therefore, the pharmaceutical composition of the present invention is useful for the therapy and prophylaxis of liver diseases [e.g. immunogenic diseases (e.g. chronic autoimmune liver diseases such as autoimmune hepatic diseases, primary biliary cirrhosis, sclerosing cholangitis, etc.), partial liver resection, acute liver necrosis (e.g. necrosis caused by toxins, viral hepatitis, shock, anoxia, etc.), hepatitis B, non-A non-B hepatitis, hepatocirrhosis, hepatic failure (e.g. fulminant hepatitis, late-onset hepatitis, “acute-on-chronic” liver failure (acute liver failure on chronic liver diseases, etc.), etc.), etc.].

The pharmaceutical composition of the present invention can be used in the form of pharmaceutical preparation, for example, in a solid, semisolid or liquid form, which contains the histone deacetylase inhibitor, such as the compound (I), as an active ingredient in admixture with an organic or inorganic carrier or excipient suitable for external, enteral or parenteral administrations. The active ingredient may be compounded, for example, with the usual non-toxic, pharmaceutically acceptable carriers for tablets, pellets, capsules, suppositories, solutions, emulsions, suspensions, injections, ointments, liniments, eye drops, lotion, gel, cream, and any other form suitable for use.

The carriers those can be used for the present invention include water, glucose; lactose, gum acacia, gelatin, mannitol, starch paste, magnesium trisilicate, talc, corn starch, keratin, colloidal silica, potato starch, urea and other carriers suitable for use in manufacturing preparations in a solid, semisolid, or liquid form. Furthermore, auxiliary, stabilizing, thickening, solubilizing and coloring agents and perfumes may be used.

For applying the composition to human, it is preferable to apply it by intravenous, intramuscular, topical or oral administration, or by a vascular stent impregnated with the compound (I). While the dosage of therapeutically effective amount of the histone deacetylase inhibitor, such as the compound (I), varies from and also depends upon the age and condition of each individual patient to be treated, when an individual patient is to be treated, in the case of intravenous administration, a daily dose of 0.01-10 mg of the histone deacetylase inhibitor, such as the compound (I), per kg weight of human being, in the case of intramuscular administration, a daily dose of 0.1-10 mg of the histone deacetylase inhibitor, such as the compound of the formula (I), per kg weight of human being, and in the case of oral administration, a daily dose of 0.5-50 mg of the histone deacetylase inhibitor, such as the compound (I), per kg weight of human being, is generally given for treatment.

During the preparation of the above-mentioned pharmaceutical administration forms, the compound (I) or a salt thereof can also be used together with other immunosuppressive substances, for example rapamycin, mycophenolic acid, cyclosporin A, tacrolimus or brequinar sodium.

Hereinafter the reactions in each Preparations and Examples for preparing the compound (I) of the present invention are explained in more detail. The invention should not be restricted by the following Preparations and Examples in any way.

The following abbreviations are also used in the present specification: HCl (hydrogen chloride); MeOH (methanol); EtOH (ethanol); IPE (diisopropyl ether); AcOH (acetic acid); AcOEt (ethyl acetate); HOBT (1-hydroxybenzotriazole); WSCD (1-ethyl-3-(3′-dimethylaminopropyl)carbodiimide); DMF (N,N-dimethylformamide); DMA (N,N-dimethylacetamide); aq. (aqueous solution); Et₃N (triethylamine); DIEA (diisopropylethylamine); NaOH (sodium hydroxide); NaH (sodium hydride); THF (tetrahydrofuran); DIBAL (diisobutylaluminiumhydride); LAH (lithium aluminium hydride); LiBH₄ (lithium borohydride); NaBH₄ (sodium borohydride); MnO₂ (manganese(IV) oxide).

### Preparation 1

To a solution of ethyl 5-chloro-6-[(2-phenoxyethyl)amino]nicotinate (1.6 g) in THF (24.0 mL) was added dropwise a solution of 0.94M DIBAL solution of hexane (15.9 mL) at 0° C. under nitrogen atmosphere and the mixture was stirred at the same temperature for 1 hour. After addition of MeOH (3.0 mL) and Potassium sodium tartrate tetrahydrate (4.2 g) at 0° C. and a mixture was stirred at ambient temperature for 1 hour. The isolated precipitate was filtered off and the solvent was removed by concentration to give {5-chloro-6-[(2-phenoxyethyl)amino]-3-pyridinyl}methanol (1.26 g).

The compounds disclosed in Preparations 2, 3, 4, 5, 6, 7, 8, 9 and 10 were obtained in a similar manner to that of Preparation 1.

### Preparation 11

A solution of (2R)-2-amino-N-benzyl-N-methylpropanamide (1.7 g) in THF (5.1 mL) was added dropwise to a mixture of LAH (1.68 g) in THF (34.0 mL) at 50° C. under nitrogen atmosphere and the mixture was stirred heated under reflux for 2 hours. After addition of water (1.68 mL), 4N-NaOH aq. (1.68 mL) and water (5.04 mL) under ice-cooling. The isolated precipitate was filtered off and the solvent was removed by concentration to give (2R)-N¹-benzyl-N¹-methyl-1,2-propanediamine (1.43 g).

The compounds disclosed in Preparations 12, 13, 14, 15, 16, 17, 18, 19, 20, 21 and 22 were obtained in a similar manner to that of Preparation 11.

### Preparation 23

LiBH₄ (1.2 g) was added a solution of ethyl 5-chloro-6-({2-[(4-fluorobenzyl)(methyl)amino]-2-oxoethyl}amino)nicotinate (3.5 g) in THF (70 mL) under ice-cooling and the mixture was stirred at ambient temperature for 40 hours. After addition of 1N-HCl aq. (60.0 mL) under ice-cooling and the mixture was adjusted to pH 9 with potassium carbonate. The mixture was extracted with AcOEt and extract layer was evaporated in vacuo. The residue was purified by column chromatography on silica gel using a mixture of chloroform and MeOH (19:1 v/v) as an eluant. The eluted fractions containing the desired product were collected and evaporated in vacuo to give 2-{[3-chloro-5-(hydroxymethyl)-2-pyridinyl]amino}-N-(4-fluorobenzyl)-N-methylacetamide (0.71 g).

The compounds disclosed in Preparations 24, 25, 26, 27 and 28 were obtained in a similar manner to that of Preparation 23.

### Preparation 29

To the mixture of ethyl 5-chloro-6-({2-[(4-fluorobenzoyl)amino]-2-methylpropyl}amino)nicotinate (1.25 g) in THF (25 mL) was added a LiBH₄ (0.84 g) under ice-cooling and the mixture was stirred at ambient temperature for 20 hours. To the reaction mixture was added dropwise a 1N-HCl aq. (44.4 mL) under ice-cooling. After a mixture was poured into a mixture of AcOEt and ice water and the mixture was adjusted to pH 9.0 with 20% aqueous potassium carbonate. The separated organic layer was washed with water, dried over magnesium sulfate and evaporated in vacuo to give N-(2-{[3-chloro-5-(hydroxymethyl)-2-pyridinyl]amino}-1,1-dimethylethyl)-4-fluorobenzamide (1.08 g).

### Preparation 30

A mixture of {5-chloro-6-[(2-phenoxyethyl)amino]-3-pyridinyl}methanol (1.2 g) and MnO₂ (3.7 g) in chloroform (24.0 mL) was stirred at 50° C. for 4 hours. The manganese oxide was filtered off and the solvent was removed by concentration. The residue was triturated with IPE and hexane to give 5-chloro-6-[(2-phenoxyethyl)amino]nicotinaldehyde (0.78 g).

The compounds disclosed in Preparations 31, 32, 33, 34, 35, 36, 37, 38, 39, 40, 41, 42, 43, 44, 45 and 46 were obtained in a similar manner to that of Preparation 30.

### Preparation 47

A mixture of (6-{[2-(4-fluorophenoxy)ethyl]amino}-3-pyridinyl)methanol (0.55 g) and MnO₂ (1.8 g) in chloroform (11.0 mL) was stirred at 60° C. for 2 hours. The manganese oxide was filtered off and the solvent was removed by concentration to give 6-{[2-(4-fluorophenoxy)ethyl]amino}nicotinaldehyde (0.53 g).

The compounds disclosed in Preparations 48 and 49 were obtained in a similar manner to that of Preparation 47.

### Preparation 50

To the mixture of ethyl 5-chloro-6-[(2-methoxyethyl)amino]nicotinate (2.0 g) and NaBH4 (1.2 g) in THF (20 mL) was added dropwise a MeOH (6.3 mL) under reflux and the mixture was stirred at the same temperature for 4 hours. The solvent was removed by concentration. The residue was added a water and extracted with AcOEt. The extract layer was washed with water, dried over magnesium sulfate and evaporated in vacuo to give (5-chloro-6-[(2-methoxyethyl)amino]-3-pyridinyl}methanol (1.46 g).

The compound disclosed in Preparation 51 was obtained in a similar manner to that of Preparation 50.

### Preparation 52

MeOH (6.4 mL) was added dropwise to a mixture of ethyl 6-({2-[(tert-butoxycarbonyl)amino]ethyl}amino)-5-chloronicotinate (5.4 g) and NaBH₄ (2.4 g) in THF (54.0 mL) at 50 to 56° C. and the mixture was stirred heated under reflux for 2.5 hours. The solvent was removed by concentration and to the residue was added a mixture of AcOEt and water. The separated organic layer was washed with water, dried over magnesium sulfate and evaporated in vacuo. The residue was purified by column chromatography on silica gel using a mixture of chloroform and AcOEt as an eluant. The eluted fractions containing the desired product were collected and evaporated in vacuo to give tert-butyl (2-{[3-chloro-5-(hydroxymethyl)-2-pyridinyl]amino}ethyl)carbamate (3.51 g).

### Preparation 53

1-(3-dimethylaminopropyl)-3-ethylcarbodiimide (0.33 g) was added to a mixture of (2E)-3-{5-[(2-phenoxyethyl)amino]-2-pyrazinyl}acrylic acid (0.5 g), O-(tetrahydro-2H-pyran-2-yl)hydroxylamine (0.25 g) and HOBT (0.28 g) in DMF (10.0 ml) and the mixture was stirred at ambient temperature for 20 hours. The reaction mixture was poured into a mixture of IPE (50 mL) and water (30 mL) and stirred for 30 minutes. The isolated precipitate was collected by filtration to give (2E)-3-{5-[(2-phenoxyethyl)amino]-2-pyrazinyl}-N-(tetrahydro-2H-pyran-2-yloxy)acrylamide (0.62 g).

### Preparation 54

1-(3-dimethylaminopropyl)-3-ethylcarbodiimide (0.35 g) was added to a mixture of (2E)-3-{5-chloro-6-[(2-phenoxyethyl)amino]-3-pyridinyl}acrylic acid (0.6 g), O-(tetrahydro-2H-pyran-2-yl) hydroxylamine (0.27 g) and HOBT (0.31 g) in DMF (9.0 ml) and the mixture was stirred at ambient temperature for 20 hours. The reaction mixture was poured into a mixture of AcOEt and water. The separated organic layer was washed with water, dried over magnesium sulfate and evaporated in vacuo to give (2E)-3-{5-chloro-6-[(2-phenoxyethyl)amino]-3-pyridinyl}-N-(tetrahydro-2H-pyran-2-yloxy)acrylamide (0.76 g).

The compounds disclosed in Preparations 55, 56, 57, 58, 59, 60, 61, 62, 63, 64, 65, 66, 67, 68, 69, 70, 71 and 75 were obtained in a similar manner to that of Preparation 54.

### Preparation 72

WSCD (0.25 g) was added to a mixture of (2E)-3-[5-chloro-6-({2-[(4-chlorobenzoyl)amino]ethyl}amino)-3-pyridinyl]acrylic acid (0.5 g), O-(tetrahydro-2H-pyran-2-yl)hydroxylamine (0.19 g) and HOBT (0.21 g) in DMF (10.0 ml) and the mixture was stirred at ambient temperature for 20 hours. The reaction mixture was poured into a mixture of AcOEt, THF and water. The separated organic layer was washed with water, dried over magnesium sulfate and evaporated in vacuo. The residue was triturated with ether to give 4-chloro-N-{2-[(3-chloro-5-{(1E)-3-oxo-3-[(tetrahydro-2H-pyran-2-yloxy)amino]-1-propen-1-yl}-2-pyridinyl)amino]ethyl}benzamide (0.55 g).

The compounds disclosed in Preparations 73 and 74 were obtained in a similar manner to that of Preparation 72.

### Preparation 76

WSCD (4.9 g) was added to a mixture of 5,6-dichloronicotinic acid (5.0 g) and N-methoxymethanamine hydrochloride (3.1 g) in dichloromethane (50 mL) and the mixture was stirred at ambient temperature for 2 hours. The reaction mixture was washed with water, dried over magnesium sulfate and evaporated in vacuo. The residue was triturated with IPE and hexane to give 5,6-dichloro-N-methoxy-N-methylnicotinamide (4.77 g).

### Preparation 77

To the stirring mixture of ethyl diethoxyphosphorylacetate, (2.67 mL) and 60% NaH (0.54 g) in THF (27 mL) was added dropwise a solution of tert-butyl {2-[(3-chloro-5-formyl-2-pyridinyl)amino]ethyl}carbamate (3.1 g) in THF (10 mL) under ice-cooling and after the mixture was stirred at ambient temperature for 2.5 hours. The reaction mixture was poured into a mixture of AcOEt and water. The separated organic layer was washed with water, dried over magnesium sulfate and evaporated in vacuo to give ethyl (2E)-3-[6-({2-[(tert-butoxycarbonyl)amino]ethyl}amino)-5-chloro-3-pyridinyl]acrylate (3.8 g).

### Preparation 78

A mixture of 5-chloro-6-[(2-phenoxyethyl)amino]nicotinaldehyde (0.70 g), malonic acid (0.53 g) and piperidine (54 mg) in pyridine (6.1 mL) was stirred at 100° C. for 4 hours. The solvent was removed by concentration and to the residue was added a mixture of AcOEt, THF and water. The separated organic layer was washed with water, dried over magnesium sulfate and evaporated in vacuo. The residue was triturated with IPE and hexane to give (2E)-3-{5-chloro-6-[(2-phenoxyethyl)amino]-3-pyridinyl}acrylic acid (0.79 g).

The compounds disclosed in Preparations 79, 80, 81, 82, 83, 84, 85, 86, 87, 88, 89, 90, 91, 92, 93 and 94 were obtained in a similar manner to that of Preparation 78.

### Preparation 95

A mixture of 6-{[2-(4-fluorophenoxy)ethyl]amino}nicotinaldehyde (0.5 g), malonic acid (0.4 g) and piperidine (41 mg) in pyridine (4.7 mL) was stirred at 100° C. for 3 hours. The solvent was removed by concentration and to the residue was added a mixture of AcOEt (5 mL), IPE (15 mL) and water (15 mL) under stirring. The isolated precipitate was collected by filtration to give (2E)-3-(6-{[2-(4-fluorophenoxy)ethyl]amino}-3-pyridinyl)acrylic acid (0.42 g).

### Preparation 96

A mixture of N-{2-[(3-chloro-5-formyl-2-pyridinyl)amino]-1,1-dimethylethyl}-4-fluorobenzamide (0.90 g), malonic acid (0.54 g) and piperidine (55 mg) in pyridine (6.2 mL) was stirred at 100° C. for 3 hours. The solvent was removed by concentration and to the residue was added a mixture of AcOEt and water. The separated organic layer was washed with water, dried over magnesium sulfate and evaporated in vacuo. The residue was triturated with IPE to give (2E)-3-[5-chloro-6-({2-[(4-fluorobenzoyl)amino]-2-methylpropyl}amino)-3-pyridinyl]acrylic acid (0.72 g).

### Preparation 97

A solution of methyl (2E)-3-(5-chloro-2-pyrazinyl)acrylate (1.0 g), (2R)-2-amino-N-(cyclohexylmethyl)butanamide (1.5 g) and Et₃N (2.11 mL) in DMA (10 mL) was stirred at 115° C. for 10 hours. The reaction mixture was poured into a mixture of AcOEt and water. The separated organic layer was washed with water, dried over magnesium sulfate and evaporated in vacuo. The residue was triturated with IPE to give methyl (2E)-3-[5-({(1R)-1-[(cyclohexylmethyl)carbamoyl]propyl}amino)pyrazin-2-yl]acrylate (1.23 g).

The compounds disclosed in Preparations 98, 99, 100, 101, 102, 103, 104, 105, 106, 107, 108, 109, 110, 111, 112, 113, 114, 115, 116, 117, 118, 119, 120, 121, 122, 123, 124, 245, 246, 247, 248, 249, 250, 251, 252, 253, 254, 255, 256, 257, 258, 259 and 260 were obtained in a similar manner to that of Preparation 97.

### Preparation 125

A solution of ethyl 5,6-dichloronicotinate (3.0 g), 2-methyl-1,2-propanediamine (1.7 g) and DIEA (5.2 mL) in 1,3-dimethyl-2-imidazolidinone (30.0 mL) was stirred at 100° C. for 3.5 hours. The reaction mixture was poured into a mixture of AcOEt and water. The separated organic layer was washed with water, dried over magnesium sulfate and evaporated in vacuo to give ethyl 6-[(2-amino-2-methylpropyl)amino]-5-chloronicotinate (3.15 g).

### Preparation 126

A solution of methyl (2E)-3-(5-chloro-2-pyrazinyl)acrylate (0.5 g), (2R)-N¹-benzyl-N¹-methyl-1,2-propanediamine (0.67 g) and Et₃N (1.05 mL) in DMA (5.0 mL) was stirred at 100° C. for 7 hours. The reaction mixture was poured into a mixture of saturated sodium hydrogen carbonate aq. and extracted with mixture of AcOEt and THF. The extract layer was washed with water, dried over magnesium sulfate and evaporated in vacuo. The residue was purified by column chromatography on silica gel using a mixture of chloroform and MeOH (19:1 v/v) as an eluant. The eluted fractions containing the desired product were collected and evaporated in vacuo to give methyl (2E)-3-[5-({(1R)-2-[benzyl(methyl)amino]-1-methylethyl}amino)-2-pyrazinyl]acrylate (0.71 g).

The compounds disclosed in Preparations 127, 128, 129, 130, 131, 132, 133, 134, 135, 136 and 137 were obtained in a similar manner to that of Preparation 126.

### Preparation 138

The solution of methyl (2E)-3-(5-chloro-2-pyrazinyl)acrylate (0.5 g), [2-(2-chlorophenoxy)ethyl]amine (0.65 g) and Et₃N (1.05 mL) in DMA (5.0 mL) was stirred at 100° C. for 5 hours. The reaction mixture was poured into a mixture of AcOEt and water. The separated organic layer was washed with water, dried over magnesium sulfate and evaporated in vacuo to give methyl (2E)-3-(5-{[2-(2-chlorophenoxy)ethyl]amino}-2-pyrazinyl)acrylate (0.72 g).

The compounds disclosed in Preparations 139, 140, 141, 142, 143, 144, 145, 146 and 147 were obtained in a similar manner to that of Preparation 138.

### Preparation 148

The mixture of methyl (2E)-3-(6-chloro-2-pyrazinyl)acrylate (0.6 g), (2-phenoxyethyl)amine (0.48 mL), cesium carbonate (1.48 g), 1,1’-binaphthalene-2,2′-diylbis(diphenylphosphine) (0.19 g) and palladium(II) acetate (34.0 mg) in dioxane (12.0 mL) was heated under reflux for 2 hours. The reaction mixture was poured into a mixture of AcOEt and water. The separated organic layer was washed with water, dried over magnesium sulfate and evaporated in, vacuo. The residue was purified by column chromatography on silica gel using a mixture of hexane and AcOEt (7:3 v/v) as an eluant. The eluted fractions containing the desired product were collected and evaporated in vacuo to give methyl (2E)-3-{6-[(2-phenoxyethyl) amino]-2-pyrazinyl}acrylate (0.75 g).

### Preparation 149

A solution of methyl (2E)-3-(5-chloro-2-pyrazinyl)acrylate (1.1 g), tert-butyl [(2R)-2-aminopropyl]carbamate (1.45 g) and Et₃N (2.32 mL) in DMA (11 mL) was stirred at 100° C. for 12 hours. The reaction mixture was poured into a mixture of AcOEt and water. The separated organic layer was washed with water, dried over magnesium sulfate and evaporated in vacuo. The residue was triturated with IPE to give methyl (2E)-3-[5-({(1R)-2-[(tert-butoxycarbonyl)amino]-1-methylethyl}amino)pyrazin-2-yl]acrylate (1.0 g).

The compounds disclosed in Preparations 150 and 151 were obtained in a similar manner to that of Preparation 149.

### Preparation 152

A solution of ethyl 5,6-dichloronicotinate (1.2 g), 2-phenoxyethanamine (0.79 mL) and potassium carbonate (2.26 mL) in DMF (12.0 mL) was stirred at 100° C. for 4 hours. The reaction mixture was poured into a mixture of AcOEt and water. The separated organic layer was washed with water, dried over magnesium sulfate and evaporated in vacuo to give ethyl 5-chloro-6-[(2-phenoxyethyl)amino]nicotinate (1.67 g).

The compounds disclosed in Preparations 153, 154, 155, 156, 157, 158, 159, 160, 161 and 162 were obtained in a similar manner to that of Preparation 152.

### Preparation 163

The mixture of ethyl 5,6-dichloronicotinate (5.0 g), N-(4-fluorobenzyl)-N-methylglycinamide hydrochloride (6.3 g) and DIEA (8.7 mL) in 1,3-dimethyl-2-imidazolidinone (50.0 mL) was stirred at 100° C. for 4.5 hours. The reaction mixture was poured into a mixture of water and extracted with AcOEt. The extract layer was washed with water, dried over magnesium sulfate and evaporated in vacuo. The residue was triturated with IPE to give ethyl 5-chloro-6-({2-[(4-fluorobenzyl)(methyl)amino]-2-oxoethyl}amino)nicotinate (7.57 g).

The compounds disclosed in Preparations 164, 165, 166, 167 and 168 were obtained in a similar manner to that of Preparation 163.

### Preparation 169

A solution of methyl (2E)-3-(5,6-dichloropyridin-3-yl)acrylate (0.5 g), Et₃N (0.9 mL) and (2R)-2-amino-N-(cyclohexylmethyl)propanamide (0.6 g) in DMA (5.0 mL) was stirred at 145° C. for 12 hours. The reaction mixture was poured into a mixture of AcOEt and water. The separated organic layer was washed with water, dried over magnesium sulfate and evaporated in vacuo. The residue was purified by column chromatography on silica gel using a mixture of dichloromethane and AcOEt (4:1 v/v) as an eluant. The eluted fractions containing the desired product were collected and evaporated in vacuo to give methyl (2E)-3-[5-chloro-6-({(1R)-2-[(cyclohexylmethyl)amino]-1-methyl-2-oxoethyl}amino)pyridin-3-yl]acrylate (0.32 g).

The compound disclosed in Preparation 170 was obtained in a similar manner to that of Preparation 169.

### Preparation 171

A solution of ethyl 5,6-dichloronicotinate (5.0 g), tert-butyl (2-aminoethyl)carbamate (4.0 g) and potassium carbonate (9.4 g) in DMF (50.0 mL) was stirred at 100° C. for 3.5 hours. The reaction mixture was poured into a mixture of AcOEt and water. The separated organic layer was washed with water, dried over magnesium sulfate and evaporated in vacuo. The residue was triturated with IPE to give ethyl 6-({2-[(tert-butoxycarbonyl)amino]ethyl}amino)-5-chloronicotinate (5.55 g).

### Preparation 172

The mixture of methyl (2E)-3-{5-[(2-phenoxyethyl)amino]-2-pyrazinyl}acrylate (0.55 g) and 1N-NaOH aq. (5.5 mL) in a solution of MeOH (11.0 mL) and THF (8.0 mL) was stirred at ambient temperature for 18 hours. The solvent was removed by concentration. The residue was added a mixture of AcOEt and brine and the mixture was adjusted to pH 5 with 1N-HCl aq. The separated organic layer was dried over magnesium sulfate and evaporated in vacuo to give (2E)-3-{5-[(2-phenoxyethyl)amino]-2-pyrazinyl}acrylic acid (0.51 g).

### Preparation 173

The mixture of ethyl (2E)-3-[5-chloro-6-({2-[(4-chlorobenzoyl)amino]ethyl}amino)-3-pyridinyl]acrylate (0.6 g) and 1N-NaOH aq. (7.3 mL) in MeOH (12 mL) was stirred at 60° C. for 2 hours. The solvent was removed by concentration. The residue was added a mixture of AcOEt and water and the mixture was adjusted to pH 5 with 1N-HCl. The separated organic layer was dried over magnesium sulfate and evaporated in vacuo. The residue was triturated with IPE to give (2E)-3-[5-chloro-6-({2-[(4-chlorobenzoyl)amino]ethyl}amino)-3-pyridinyl]acrylic acid (0.52 g).

The compounds disclosed in Preparations 174 and 175 were obtained in a similar manner to that of Preparation 173.

### Preparation 176

The mixture of methyl (2E)-3-[5-({(1R)-1-[(cyclohexylmethyl)carbamoyl]propyl}amino)pyrazin-2-yl]acrylate (1.2 g) and 1N-NaOH aq. (8.3 mL) in MeOH (24 mL) was stirred at 60° C. for 2.5 hours. To the reaction mixture was neutralized with 1N—HCl aq. (8.3 mL) and the mixture was evaporated in vacuo. To the residue in DMF (12 ml) was added O-(tetrahydro-2H-pyran-2-yl)hydroxylamine (0.59 g), HOBT (0.68 g) and WSCD (0.78 g) and the mixture was stirred at ambient temperature for 20 hours. The reaction mixture was poured into a mixture of IPE and 2% sodium hydrogen carbonate aq. under stirring. The isolated precipitate was collected by filtration to give (2R)-N-(cyclohexylmethyl)-2-[(5-{(1E)-3-oxo-3-[(tetrahydro-2H-pyran-2-yloxy)amino]prop-1-en-1-yl}pyrazin-2-yl)amino]butanamide (0.82 g).

The compounds disclosed in Preparations 177, 178, 179, 180, 181, 182, 183, 184, 185, 186, 187, 188, 189, 190, 191, 192, 193, 194, 195, 196, 197, 198, 261, 262, 263, 264, 265, 266, 267, 268, 269, 270, 271, 272, 273, 274, 275 and 276 were obtained in a similar manner to that of Preparation 176.

### Preparation 199

The mixture of methyl (2E)-3-[5-({(1R)-1-[benzylcarbamoyl]-4-methylpentyl}amino)pyrazin-2-yl]acrylate (1.1 g) and 1N—NaOH aq. (29 mL) in MeOH (60 mL) was stirred at 60° C. for 3 hours. The reaction mixture was neutralized with 1N—HCl aq. (29 mL) and evaporated under reduced pressure. The residue was extracted twice with chloroform. Combined organic layer was dried over magnesium sulfate, filtered and evaporated. To the residue in DMF (20 ml) was added O-(tetrahydro-2H-pyran-2-yl)hydroxylamine (404 mg), HOBT (388 mg) and WSCD (670 mg) and the mixture was stirred at ambient temperature for 12 hours. A mixture of ethyl acetate and water was poured into the reaction mixture. Aqueous layer was separated and extracted twice with AcOEt. The combined organic layer was washed twice with water, dried over magnesium sulfate, filtered and evaporated. The residue was column chromatographed by Yamazen packed column (35×100 mm, chloroform/AcOEt) to give (2R)-N-benzyl-2-[(5-{(1E)-3-oxo-3-[(tetrahydro-2H-pyran-2-yloxy)amino]prop-1-en-1-yl)pyrazin-2-yl)amino]-4-methyl pentanamide (883 mg) as amorphous.

The compounds disclosed in Preparations 200, 201, 202 and 203 were obtained in a similar manner to that of Preparation 199.

### Preparation 204

The mixture of methyl (2E)-3-[5-({(1R)-2-[benzyl(methyl)amino]-1-methylethyl}amino)-2-pyrazinyl]acrylate (0.6 g) and 1N—NaOH aq. (3.5 mL) in MeOH (12 mL) was stirred at 55° C. for 2.5 hours. To the reaction mixture was neutralized with 1N—HCl aq. (3.5 mL) and the mixture was evaporated in vacuo. To the residue in DMF (10 ml) was added O-(tetrahydro-2H-pyran-2-yl)hydroxylamine (0.31 g), HOBT (0.36 g) and WSCD (0.41 g) and the mixture was stirred at ambient temperature for 20 hours. The reaction mixture was poured into water and extracted with mixture of AcOEt and THF. The extract layer was washed with water, dried over magnesium sulfate and evaporated in vacuo. The residue was purified by column chromatography on silica gel using a mixture of chloroform and MeOH (19:1 v/v) as an eluant. The eluted fractions containing the desired product were collected and evaporated in vacuo to give (2E)-3-[5-({(1R)-2-[benzyl(methyl)amino]-1-methylethyl}amino)-2-pyrazinyl]-N-(tetrahydro-2H-pyran-2-yloxy)acrylamide (0.71 g).

The compounds disclosed in Preparations 205, 206, 207, 208, 209, 210, 211, 212, 213, 214 and 215 were obtained in a similar manner to that of Preparation 204.

### Preparation 216

The mixture of methyl (2E)-3-(5-{[2-(2-chlorophenoxy)ethyl]amino}-2-pyrazinyl)acrylate (0.7 g) and 1N—NaOH aq. (4.2 mL) in a solution of MeOH (7.0 mL) and THF (7.0 mL) was stirred at 50° C. for 1 hour. To the reaction mixture was neutralized with 1N—HCl aq. (4.2 mL) and the mixture was evaporated in vacuo.

To the residue in DMF (10.5 ml) was added O-(tetrahydro-2H-pyran-2-yl)hydroxylamine (0.37 g), HOBT (0.43 g) and WSCD (0.49 g) and the mixture was stirred at ambient temperature for 20 hours. The reaction mixture was poured into a mixture of AcOEt and water. The separated organic layer was washed with water, dried over magnesium sulfate and evaporated in vacuo. The residue was triturated with IPE to give (2E)-3-(5-{[2-(2-chlorophenoxy)ethyl]amino}-2-pyrazinyl)-N-(tetrahydro-2H-pyran-2-yloxy)acrylamide (0.7 g)

The compounds disclosed in Preparations 217, 218, 219, 220, 221, 222, 223, 224 and 225 were obtained in a similar manner to that of Preparation 216.

### Preparation 226

The mixture of methyl (2E)-3-[5-({(1R)-2-[(4-chlorobenzoyl)amino]-1-methylethyl}amino)pyrazin-2-yl]acrylate (0.47 g) and 1N—NaOH aq. (3.8 mL) in MeOH (9.4 mL) was stirred at 60° C. for 2.5 hours. To the reaction mixture was neutralized with 1N—HCl aq. (3.8 mL) and the mixture was evaporated in vacuo. To the residue in DMF (10 ml) was added O-(tetrahydro-2H-pyran-2-yl)hydroxylamine (0.22 g), HOBT (0.25 g) and WSCD (0.29 g) and the mixture was stirred at ambient temperature for 20 hours. The reaction mixture was poured into a 2% sodium hydrogen carbonate aq. and extracted with a solution of AcOEt and THF. The extract layer was washed with brine, dried over magnesium sulfate and evaporated in vacuo. The residue was purified by column chromatography on silica gel using a mixture of AcOEt and THF (9:1 v/v) as an eluant. The eluted fractions containing the desired product were collected and evaporated in vacuo to give 4-chloro-N-{(2R)-2-[(5-{(1E)-3-oxo-3-[(tetrahydro-2H-pyran-2-yloxy)amino]prop-1-en-1-yl}pyrazin-2-yl)amino]propyl}benzamide (0.45 g).

The compounds disclosed in Preparations 227, 228, 229 and 230 were obtained in a similar manner to that of Preparation 226.

### Preparation 231

The mixture of methyl (2E)-3-[5-chloro-6-({(1R)-2-[(cyclohexylmethyl)amino]-1-methyl-2-oxoethyl}amino)pyridin-3-yl]acrylate (0.45 g) and 4N—NaOH aq. (0.89 mL) in MeOH (9.0 mL) was stirred at 55° C. for 3.5 hours. To the reaction mixture was neutralized with 1N—HCl aq. (3.55 mL) and the mixture was evaporated in vacuo. To the residue in DMF (9.0 ml) was added O-(tetrahydro-2H-pyran-2-yl)hydroxylamine (0.21 g), HOBT (0.24 g) and WSCD (0.28 g) and the mixture was stirred at ambient temperature for 20 hours. The reaction mixture was poured into a mixture of AcOEt and water. The separated organic layer was washed with water, dried over magnesium sulfate and evaporated in vacuo. The residue was purified by column chromatography on silica gel using a mixture of AcOEt and hexane (3:1 v/v) as an eluant. The eluted fractions containing the desired product were collected and evaporated in vacuo to give N²-(3-chloro-5-{(1E)-3-oxo-3-[(tetrahydro-2H-pyran-2-yloxy)amino]prop-1-en-1-yl}pyridin-2-yl)-N¹-(cyclohexylmethyl)-D-alaninamide (0.33 g).

The compound disclosed in Preparation 232 was obtained in a similar manner to that of Preparation 231.

### Preparation 233

1) To a solution of 5,6-dichloro-N-methoxy-N-methylnicotinamide (4.7 g) in toluene (141.0 mL) was added dropwise a solution of 0.99M diisobutylaluminium hydride solution of toluene (22.2 mL) at −30° C. under nitrogen atmosphere and the mixture was stirred at the same temperature for 30 minutes. The reaction mixture was quenched with MeOH (4.1 mL) and stirred at 0° C. for 30 minutes. (Solution A)

2) To a solution of methyl (dimethoxyphosphoryl)acetate (3.4 mL) in toluene (103 mL) was added portionwise 60% NaH (0.96 g) at 20 to 30° C. under nitrogen atmosphere and the mixture was stirred at the same temperature for 30 minutes. To the mixture was added dropwise above Solution A at 0 to 10° C. and the mixture was stirred at ambient temperature for 1 hour. The reaction mixture was poured into a mixture of AcOEt and water and adjusted to pH 2 with 1N—HCl aq. The separated organic layer was washed with saturated sodium hydrogen carbonate aq., dried over magnesium sulfate and evaporated in vacuo. The residue was triturated with IPE to give methyl (2E)-3-(5,6-dichloropyridin-3-yl)acrylate (2.83 g).

### Preparation 234

1-(3-dimethylaminopropyl)-3-ethylcarbodiimide (0.31 g) was added a mixture of methyl (2E)-3-(5-{[(1R)-2-amino-1-methylethyl]amino}pyrazin-2-yl)acrylate dihydrochloride (0.52 g), Et₃N (0.47 mL), 4-chlorobenzoic acid (0.32 g), and HOBT (0.27 g) in DMF (5.0 mL) and the mixture was stirred at ambient temperature for 18 hours. The reaction mixture was poured into a water and IPE and isolated precipitate was collected by filtration to give methyl (2E)-3-[5-({(1R)-2-[(4-chlorobenzoyl)amino]-1-methylethyl}amino)pyrazin-2-yl]acrylate (0.48 g).

The compounds disclosed in Preparations 235 and 236 were obtained in a similar manner to that of Preparation 234.

### Preparation 237

1-(3-dimethylaminopropyl)-3-ethylcarbodiimide (0.27 g) was added a mixture of methyl (2E)-3-(5-{[2-(benzylamino)ethyl]amino}-2-pyrazinyl)acrylate (0.50 g), AcOH (96 mg), and HOBT (0.24 g) in dichloromethane (10.0 mL) and the mixture was stirred at ambient temperature for 20 hours. The reaction mixture was poured into a water and extracted with dichloromethane. The extract layer was washed with water, dried over magnesium sulfate and evaporated in vacuo. The residue was triturated with ether to give methyl (2E)-3-[5-({2-[acetyl(benzyl)amino]ethyl}amino)-2-pyrazinyl]acrylate (0.47 g).

### Preparation 238

1-(3-dimethylaminopropyl)-3-ethylcarbodiimide (0.50 g) was added a mixture of ethyl (2E)-3-{6-[(2-aminoethyl)amino]-5-chloro-3-pyridinyl}acrylate dihydrochloride (1.0 g), 4-chlorobenzoic acid (0.5 g), Et₃N (0.85 mL) and HOBT (0.43 g) in DMF (20.0 mL) and the mixture was stirred at ambient temperature for 20 hours. The reaction mixture was poured into a mixture of saturated sodium hydrogen carbonate aq. and AcOEt. The separated organic layer was washed with water, dried over magnesium sulfate and evaporated in vacuo. The residue was triturated with IPE and hexane to give ethyl (2E)-3-[5-chloro-6-({2-[(4-chlorobenzoyl)amino]ethyl}amino)-3-pyridinyl]acrylate (1.18 g).

The compounds disclosed in Preparations 239 and 240 were obtained in a similar manner to that of Preparation 238.

### Preparation 241

4-fluorobenzoyl chloride (0.44 mL) was added dropwise to a mixture of ethyl 6-[(2-amino-2-methylpropyl)amino]-5-chloronicotinate (1.0 g) and Et3N (0.62 mL) in dichloromethane (10.0 mL) under ice-cooling and the mixture was stirred at the same temperature for 1.5 hours. The reaction mixture was poured into a mixture of saturated sodium hydrogen carbonate aq. and chloroform. The separated organic layer was washed with water, dried over magnesium sulfate and evaporated in vacuo. The residue was purified by column chromatography on silica gel using a mixture of hexane and AcOEt (1:1 v/v) as an eluant. The eluted fractions containing the desired product were collected and evaporated in vacuo to give ethyl 5-chloro-6-({2-[(4-fluorobenzoyl)amino]-2-methylpropyl}amino)nicotinate (1.30 g).

### Preparation 242

To a mixture of ethyl 5-chloro-6-{[2-(4-fluorophenoxy)ethyl]amino}nicotinate (1.5 g) and Et₃N (0.68 mL) in a solution of MeOH (15.0 mL) and THF (10.0 mL) was added 10% palladium-on-charcoal (1.5 g, 50% wet). The reaction mixture was stirred at ambient temperature for 6 hours under hydrogen atmosphere. The catalyst was filtered off and the solvent was removed by concentration. To the residue was added a mixture of AcOEt and water. The separated organic layer was washed with water, dried over magnesium sulfate and evaporated in vacuo. The residue was triturated with IPE and hexane to give ethyl 6-{[2-(4-fluorophenoxy)ethyl]amino}nicotinate (0.78 g).

### Preparation 243

To a solution of methyl (2E)-3-[5-({(1R)-2-[(tert-butoxycarbonyl)amino]-1-methylethyl}amino)pyrazin-2-yl]acrylate (0.96 g) in MeOH (4.8 mL) was added a 4N—HCl in AcOEt (14.3 mL) and the mixture was stirred at ambient temperature for 5 hours. After addition of AcOEt (48 mL) and isolated precipitate was collected by filtration to give methyl (2E)-3-(5-{[(1R)-2-amino-1-methylethyl]amino}pyrazin-2-yl)acrylate dihydrochloride (0.80 g).

### Preparation 244

To a solution of ethyl (2E)-3-[6-({2-[(tert-butoxycarbonyl)amino]ethyl}amino)-5-chloro-3-pyridinyl]acrylate (3.8 g) in EtOH (38.0 mL) was added a 4N—HCl in AcOEt (25.7 mL) and the mixture was stirred at ambient temperature for 4 hours. After addition of IPE (100 mL) and isolated precipitate was collected by filtration to give ethyl (2E)-3-{6-[(2-aminoethyl)amino]-5-chloro-3-pyridinyl}acrylate dihydrochloride (3.2 g).

### Preparation 277

A solution of ethyl 2,6-dichloro-5-fluoronicotinate (820 mg), N-(cyclohexylmethyl)-D-valinamide (914 mg) and Et₃N (1.44 mL) in DMA (8.2 mL) was stirred at 90° C. for 5 hours. The reaction mixture was poured into a mixture of AcOEt and water. The separated organic layer was washed with 7% aqueous sodium chloride, dried over anhydrous magnesium sulfate, filtered and evaporated under reduced pressure. The residue was column chromatographed by high performanced liquid chromatography (Yamazen packed Hi-Flash column, 26×150 mm (Silica gel), hexane/AcOEt=90/10 to 40/60) to give ethyl 2-chloro-6-({(1R)-1-[(cyclohexylmethyl)carbamoyl]-2-methylpropyl}amino)-5-fluoronicotinate (980 mg).

### Preparation 278

Under nitrogen atmosphere, a solution of ethyl 2-chloro-6-({(1R)-1-[(cyclohexylmethyl)carbamoyl]-2-methylpropyl}amino)-5-fluoronicotinate (970 mg), ammonium formate (1.03 g) and palladium-10 wt. % on activated carbon (50% water) (300 mg) in EtOH (19 mL) was refluxed with stirring for 45 minutes. The reaction mixture was filtered, evaporated under reduced pressure, and poured into a mixture of AcOEt and water. The separated organic layer was washed with 5% aqueous sodium chloride, dried over anhydrous magnesium sulfate, filtered and evaporated under reduced pressure to give ethyl 6-({(1R)-1-[(cyclohexylmethyl)carbamoyl]-2-methylpropyl}amino)-5-fluoronicotinate (910 mg).

### Preparation 279

Ethyl 6-({(1R)-1-[(cyclohexylmethyl)carbamoyl]-2-methylpropyl}amino)-5-fluoronicotinate (300 mg) was dissolved in a mixed solvent of THF (2.4 ml) and MeOH (1.2 ml). 1M-NaOH aq. (1.58 mL) was added to the solution at ambient temperature. The mixture was stirred at 50° C. for 1.5 hour. The reaction mixture was evaporated under reduced pressure, the resulting residue was poured into a mixture of water, AcOEt and THF. The pH of the aqueous layer was adjusted to ca.2 with 1M-HCl aq. The organic layer was separated, washed with 5% aqueous sodium chloride, dried over anhydrous magnesium sulfate and evaporated under reduced pressure to give 6-({(1R)-1-[(cyclohexylmethyl)carbamoyl]-2-methylpropyl}amino)-5-fluoronicotinic acid (265 mg).

### Preparation 280

Under atmospheric pressure of nitrogen, isobutyl chlorocarbonate (0.12 ml) was added dropwise to a solution of 6-({(1R)-1-[(cyclohexylmethyl)carbamoyl]-2-methylpropyl}amino)-5-fluoronicotinic acid (260 mg) and 4-methylmorpholine (0.122 ml) in 1,2-dimethoxyethane (2.6 ml) with stirring below 0° C., and the reaction mixture was stirred below 0° C. for 30 minutes. Insoluble material was removed by filtration, and a suspension of NaBH₄ (98 mg) in water (2 ml) was added to the filtrate below 0° C. at one portion, and the mixture was stirred at ambient temperature for 30 mimutes. A suspension of NaBH₄ (80 mg) in water (1.5 ml) was added to it again. The reaction mixture was stirred at ambient temperature for 30 mimutes, and poured into a mixed solution of water, AcOEt and THF. The pH of the aqueous layer was adjusted to ca.2 with 1M-HCl aq. The organic layer was separated, washed with 10% aqueous sodium chloride, dried over anhydrous magnesium sulfate and evaporated under reduced pressure. The residue was column chromatographed by high performanced liquid chromatography (Yamazen packed Hi-Flash column, 20×65 mm (Silica gel), chloroform/MeOH=94/6 to 88/12) to give N¹-(cyclohexylmethyl)-N²-[3-fluoro-5-(hydroxymethyl)pyridin-2-yl]-D-valinamide (255 mg).

### Preparation 281

To a solution of N¹-(cyclohexylmethyl)-N²-[3-fluoro-5-(hydroxymethyl)pyridin-2-yl]-D-valinamide (245 mg) in AcOEt (5.6 mL) was added activated MnO₂ (568 mg) at ambient temperature. After stirring at 70° C. for 2 hours, activated MnO₂ (140 mg) was added to the mixture, and it was stirred at 70° C. for 1 hour. After cooling, anhydrous magnesium sulfate was added to the reaction mixture, and it was stirred at ambient temperature for 10 minutes. Insoluble material was removed by filtration, washed with AcOEt, chloroform. The filtrate and washings were combined, and evaporated under reduced pressure. The resulting residue was evaporated with toluene in vacuo to give syrup.

On the other hand, to an ice-cooled suspension of 60% sodium hydride (33.4 mg) in THF (4 ml) was added a solution of ethyl (diethoxyphosphoryl)acetate (0.16 ml) in THF (1 ml), then the mixture was stirred at ambient temperature for 15 minutes. The above syrup was added to the mixture at ambient temperature, the reaction mixture was stirred at ambient temperature for 2 hours. The mixture was poured into a mixture of AcOEt and 5% aqueous sodium chloride. The separated organic layer was washed with brine, dried over anhydrous magnesium sulfate and evaporated under reduced pressure. The residue was column chromatographed by high performanced liquid chromatography (Yamazen packed Hi-Flash column, 20×65 mm (Silica gel), hexane/AcOEt=86/14 to 34/66) to give ethyl (2E)-3-[6-({(1R)-1-[(cyclohexylmethyl)carbamoyl]-2-methylpropyl}amino)-5-fluoropyridin-3-yl]acrylate (247 mg).

### Preparation 282

To a solution of ethyl (2E)-3-[6-({(1R)-1-[(cyclohexylmethyl)carbamoyl]-2-methylpropyl}amino)-5-fluoropyridin-3-yl]acrylate (240 mg) in a mixed solvent of MeOH (0.96 mL) and THF (1.92 ml) was added 1M-NaOH aq. (1.18 mL) at ambient temperature, and the mixture was stirred at 50° C. for 1.5 hours. The reaction mixture was neutralized with 1M-HCl aq. (1.18 mL) and evaporated under reduced pressure. The residue was poured into a mixture of AcOEt, THF, and 5% aqueous sodium chloride. The separated organic layer was washed with brine, dried over anhydrous magnesium sulfate and evaporated under reduced pressure. To the residue in DMF (3.6 ml) were added O-(tetrahydro-2H-pyran-2-yl)hydroxylamine (104 mg), HOBT (120 mg) and WSCD (138 mg) at ambient temperature, and the mixture was stirred at ambient temperature for 62 hours. The reaction mixture was poured into a mixture of AcOEt and water. The separated organic layer was washed with 10% aqueous sodium chloride, dried over anhydrous magnesium sulfate, filtered and evaporated under reduced pressure. The residue was column chromatographed by high performanced liquid chromatography (Yamazen packed Hi-Flash column, 20×65 mm (Silica gel), hexane/AcOEt=50/50 to 10/90) to give colorless foam. The obtained foam was triturated with AcOEt to give N¹-(cyclohexylmethyl)-N²-(3-fluoro-5-{(1E)-3-oxo-3-[(tetrahydro-2H-pyran-2-yloxy)amino]prop-1-en-1-yl}pyridin-2-yl)-D-valinamide (229 mg).

TABLE 2
Preparation number and chemical structure
Pr: Preparation number; Str.: chemical structure;

| Pr | Str. |
|---|---|
| 1 | 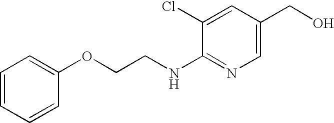 |
| 2 | 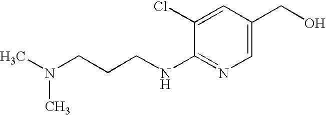 |
| 3 | 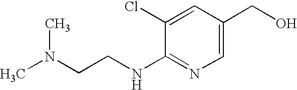 |
| 4 | 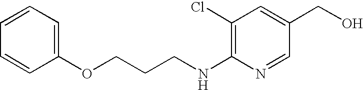 |
| 5 | 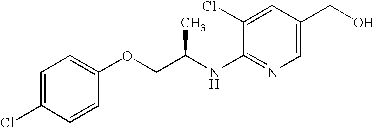 |
| 6 | 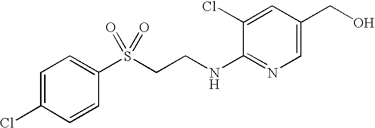 |
| 7 | 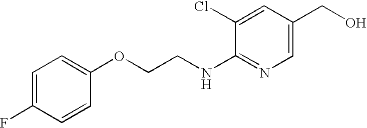 |
| 8 | 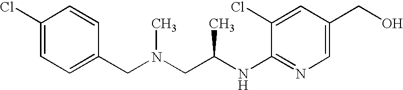 |
| 9 | 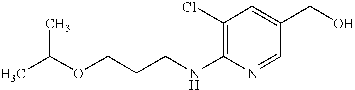 |
| 10 | 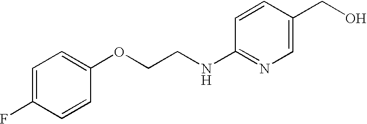 |
| 11 | 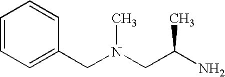 |
| 12 | 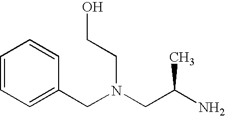 |
| 13 | 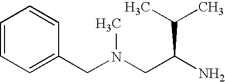 |
| 14 | 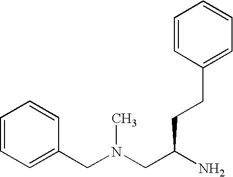 |
| 15 | 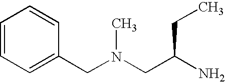 |
| 16 | 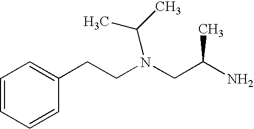 |
| 17 | 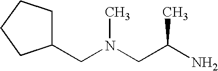 |
| 18 | 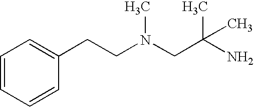 |
| 19 | 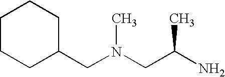 |
| 20 | 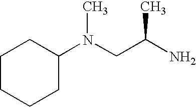 |
| 21 | 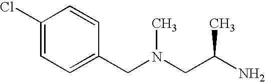 |
| 22 | 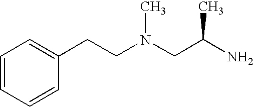 |
| 23 | 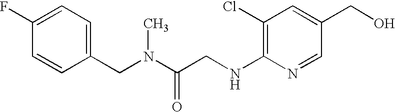 |
| 24 | 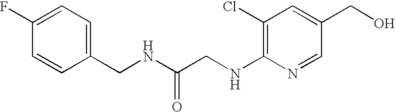 |
| 25 | 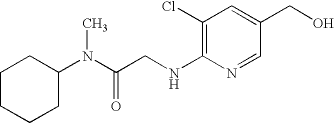 |
| 26 | 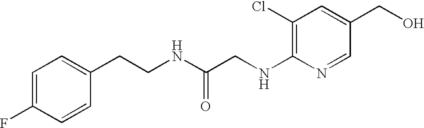 |
| 27 | 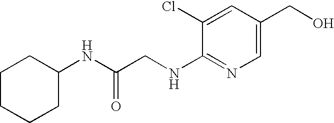 |
| 28 | 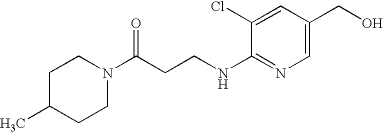 |
| 29 | 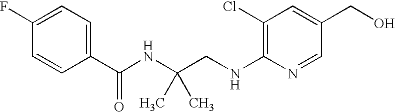 |
| 30 | 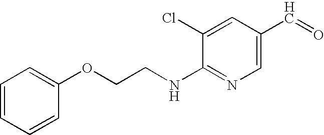 |
| 31 | 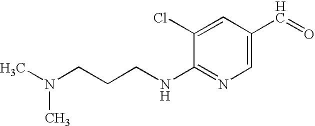 |
| 32 | 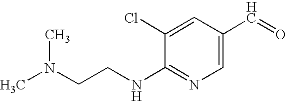 |
| 33 | 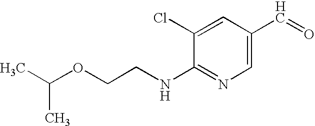 |
| 34 | 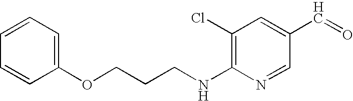 |
| 35 | 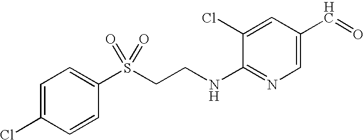 |
| 36 | 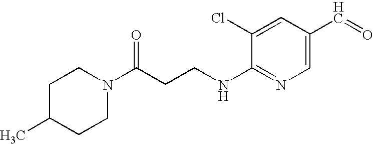 |
| 37 | 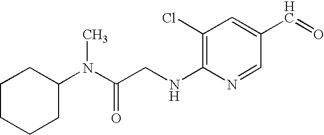 |
| 38 | 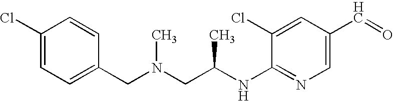 |
| 39 | 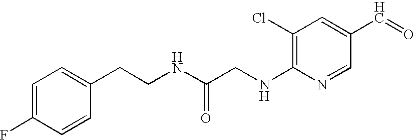 |
| 40 | 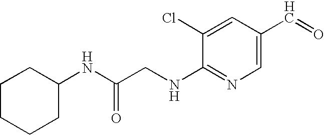 |
| 41 | 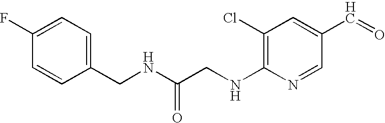 |
| 42 | 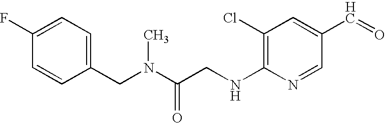 |
| 43 | 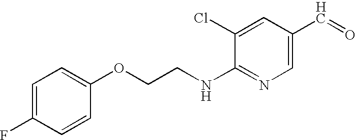 |
| 44 | 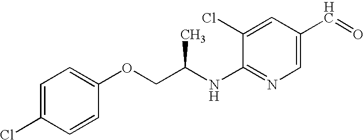 |
| 45 | 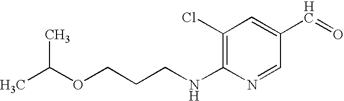 |
| 46 | 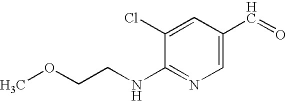 |
| 47 | 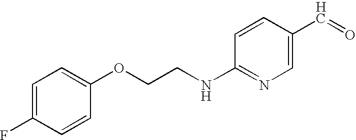 |
| 48 | 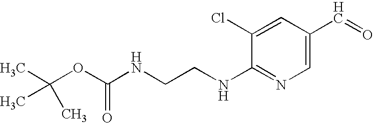 |
| 49 | 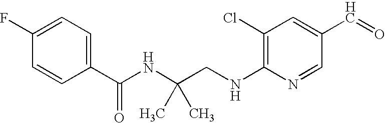 |
| 50 | 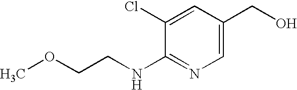 |
| 51 | 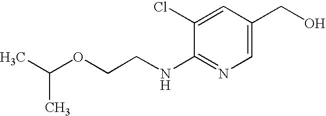 |
| 52 | 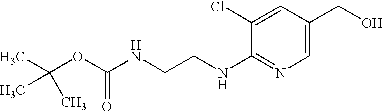 |
| 53 | 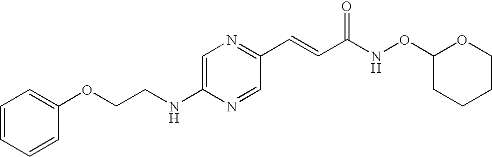 |
| 54 | 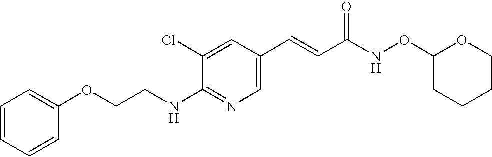 |
| 55 | 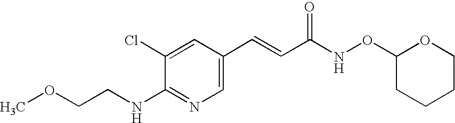 |
| 56 | 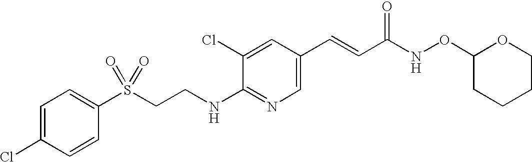 |
| 57 | 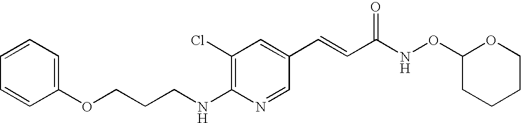 |
| 58 | 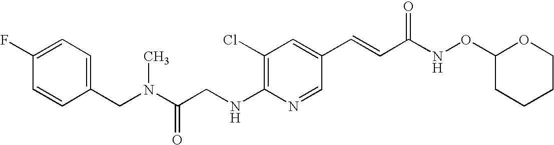 |
| 59 | 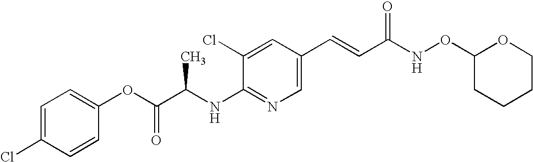 |
| 60 | 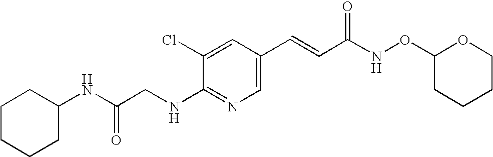 |
| 61 | 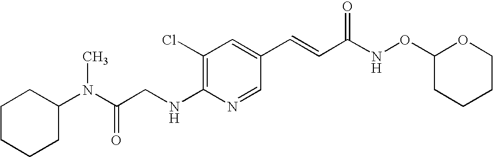 |
| 62 | 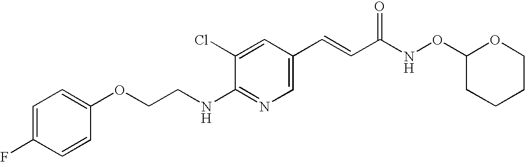 |
| 63 | 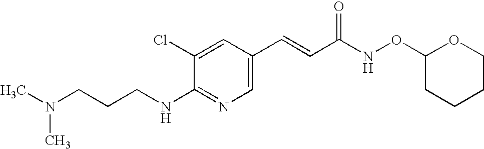 |
| 64 | 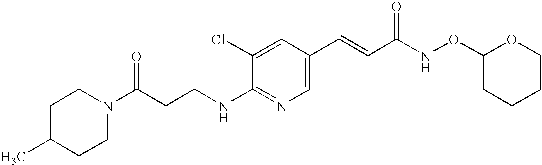 |
| 65 | 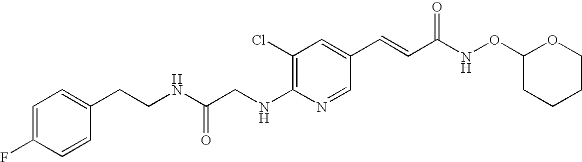 |
| 66 | 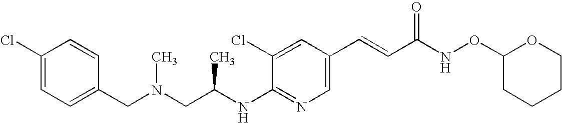 |
| 67 | 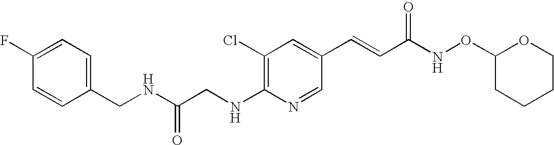 |
| 68 | 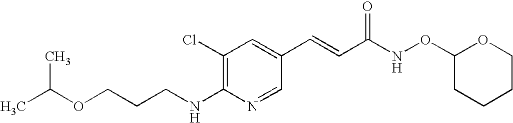 |
| 69 | 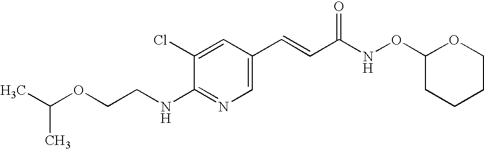 |
| 70 | 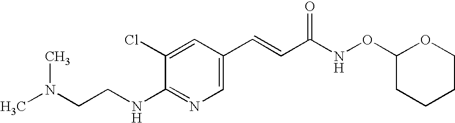 |
| 71 | 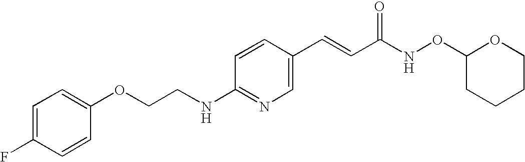 |
| 72 | 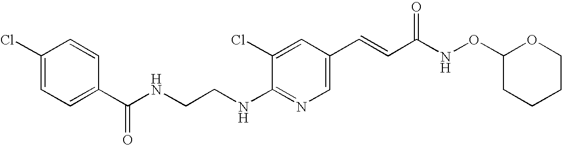 |
| 73 | 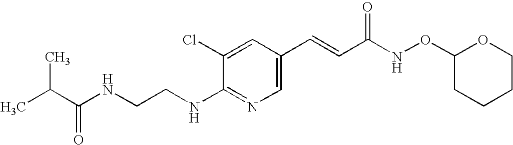 |
| 74 | 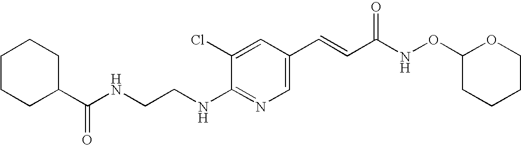 |
| 75 | 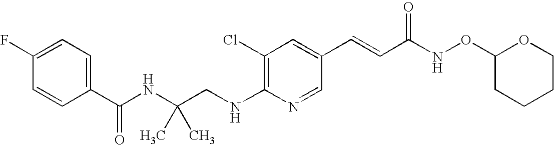 |
| 76 | 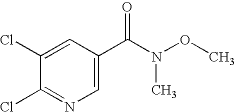 |
| 77 | 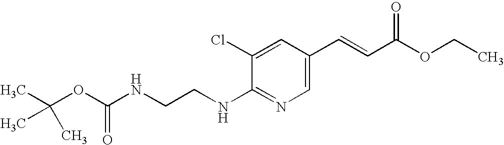 |
| 78 | 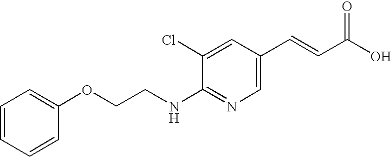 |
| 79 | 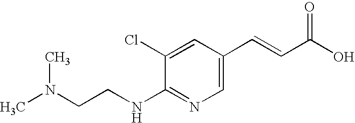 |
| 80 | 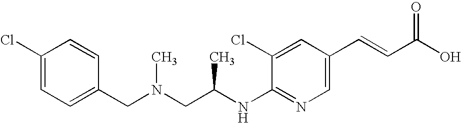 |
| 81 | 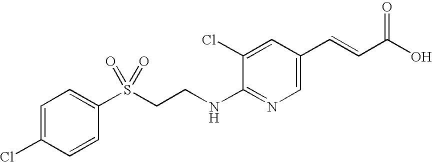 |
| 82 | 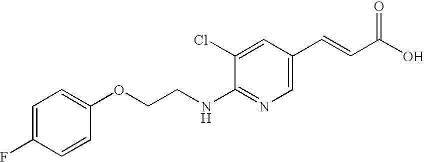 |
| 83 | 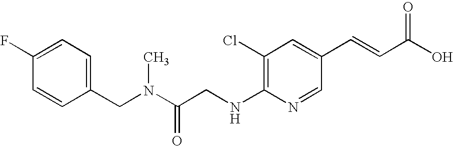 |
| 84 | 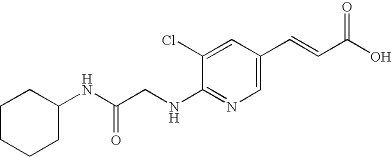 |
| 85 | 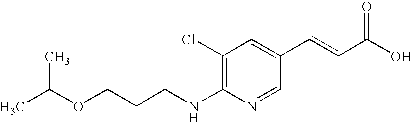 |
| 86 | 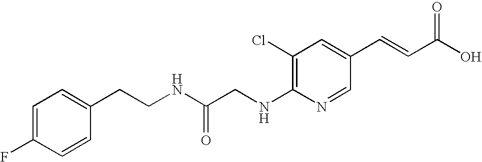 |
| 87 | 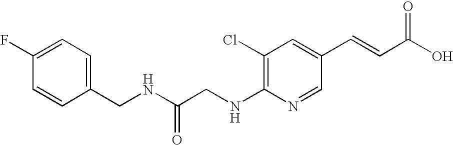 |
| 88 | 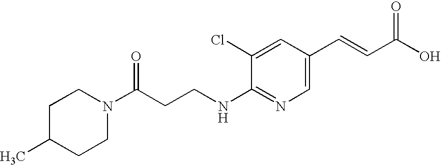 |
| 89 | 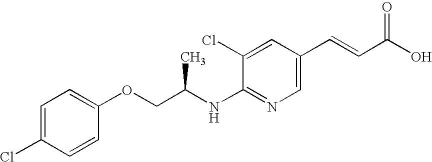 |
| 90 | 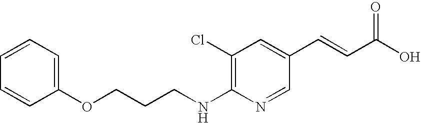 |
| 91 | 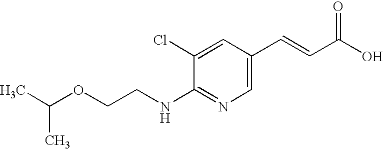 |
| 92 | 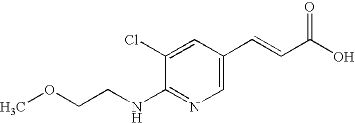 |
| 93 | 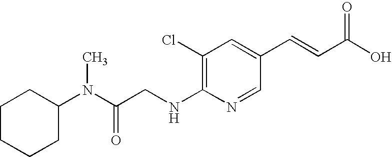 |
| 94 | 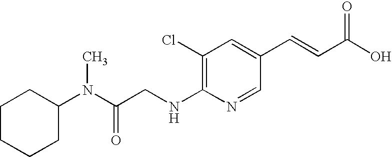 |
| 95 | 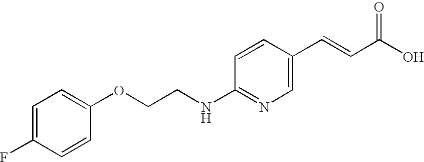 |
| 96 | 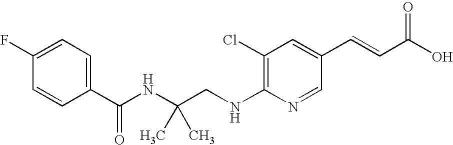 |
| 97 | 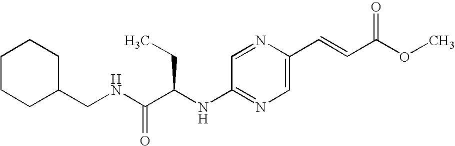 |
| 98 | 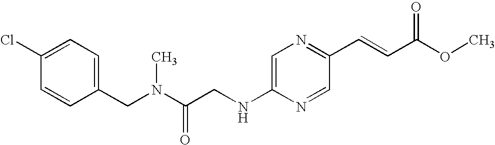 |
| 99 | 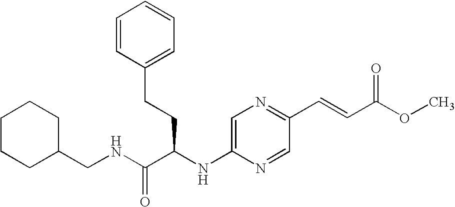 |
| 100 | 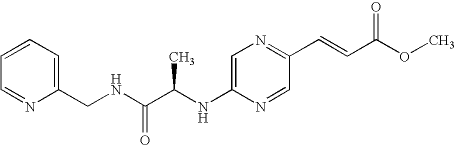 |
| 101 | 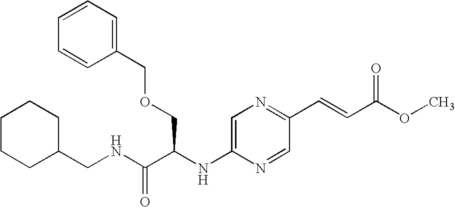 |
| 102 | 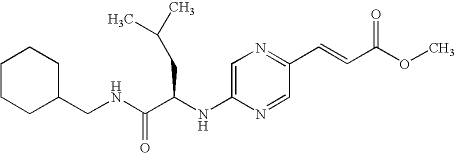 |
| 103 | 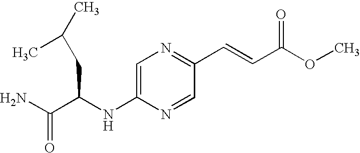 |
| 104 | 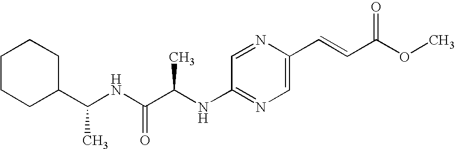 |
| 105 | 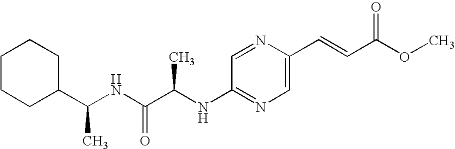 |
| 106 | 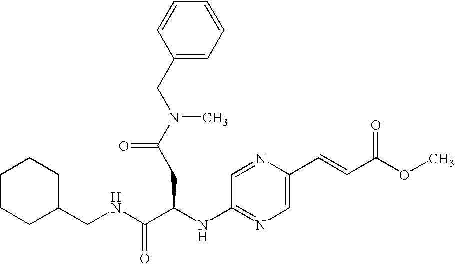 |
| 107 | 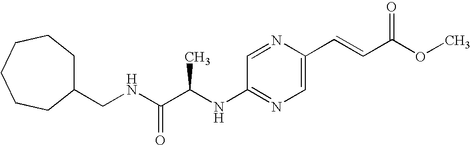 |
| 108 | 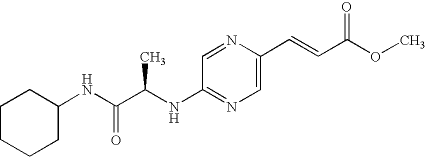 |
| 109 | 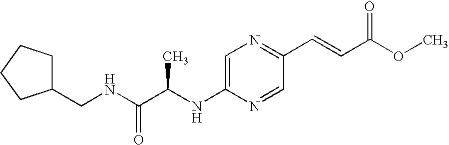 |
| 110 | 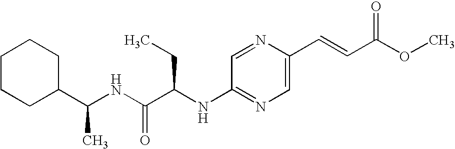 |
| 111 | 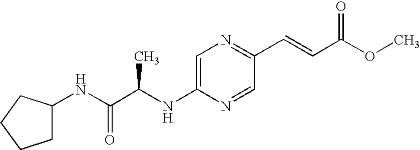 |
| 112 | 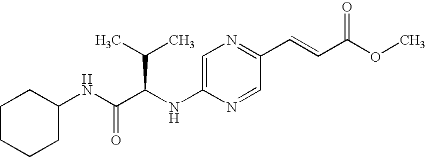 |
| 113 | 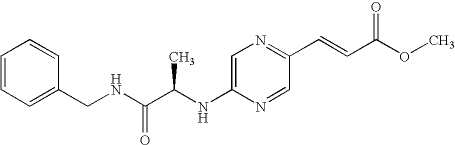 |
| 114 | 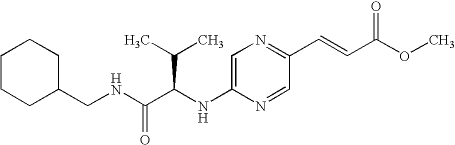 |
| 115 | 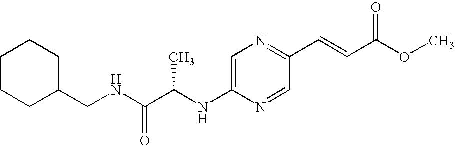 |
| 116 | 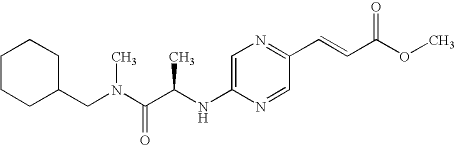 |
| 117 | 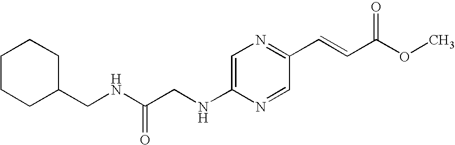 |
| 118 | 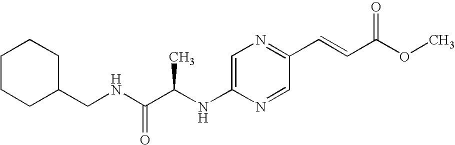 |
| 119 | 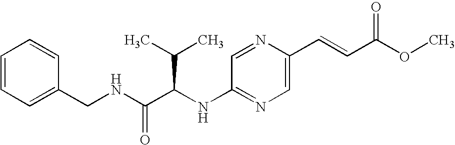 |
| 120 | 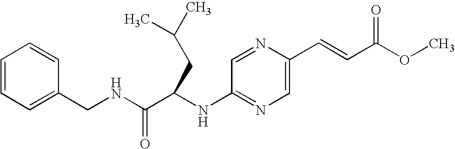 |
| 121 | 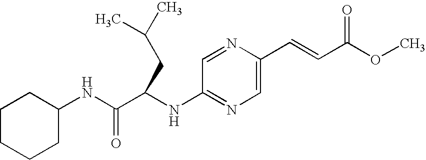 |
| 122 | 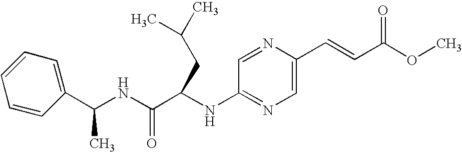 |
| 123 | 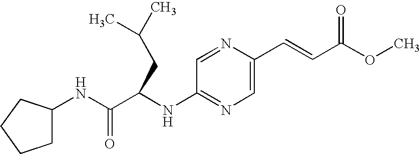 |
| 124 | 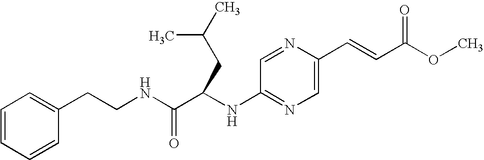 |
| 125 | 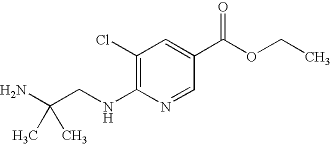 |
| 126 | 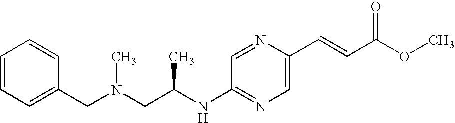 |
| 127 | 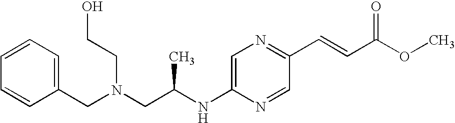 |
| 128 | 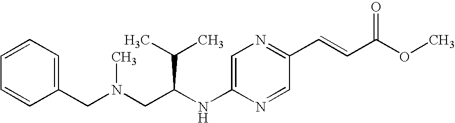 |
| 129 | 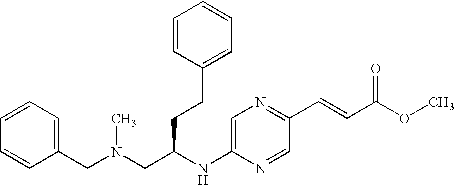 |
| 130 | 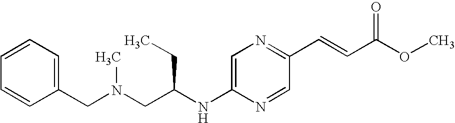 |
| 131 | 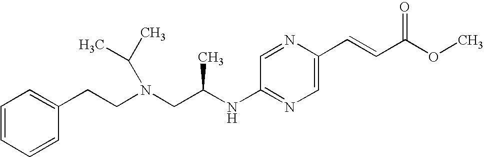 |
| 132 | 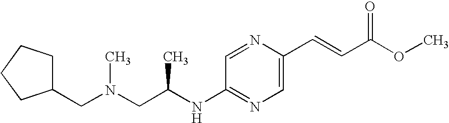 |
| 133 | 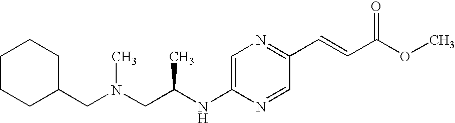 |
| 134 | 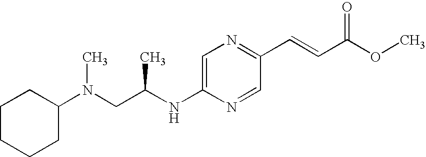 |
| 135 | 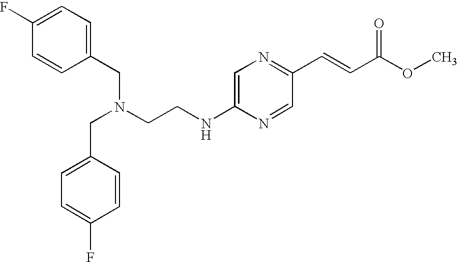 |
| 136 | 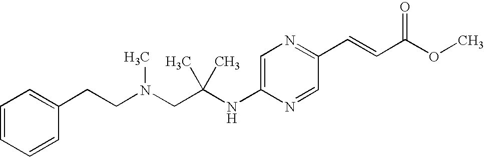 |
| 137 | 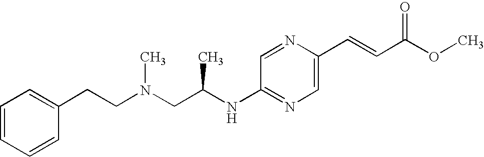 |
| 138 | 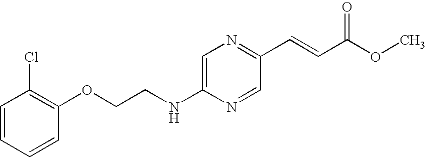 |
| 139 | 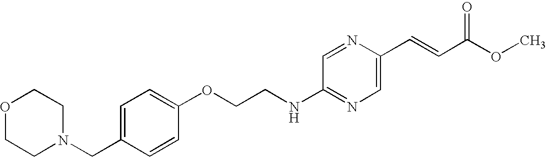 |
| 140 | 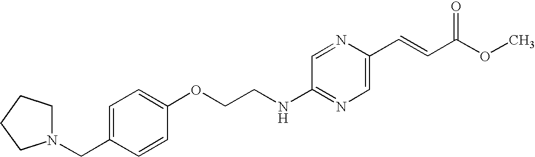 |
| 141 | 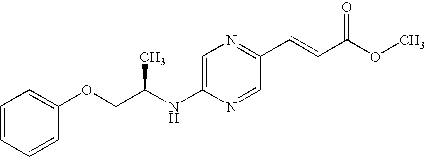 |
| 142 | 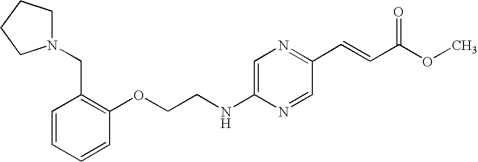 |
| 143 | 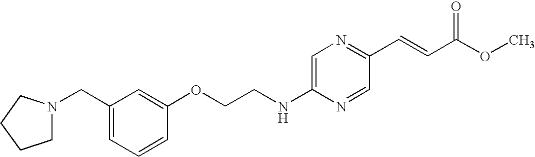 |
| 144 | 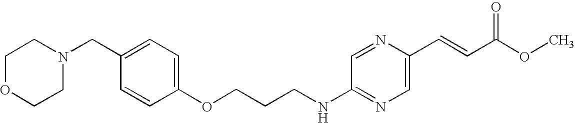 |
| 145 | 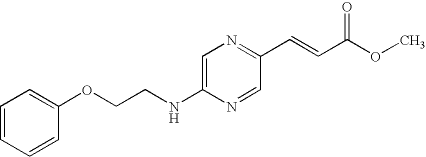 |
| 146 | 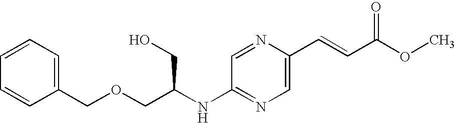 |
| 147 | 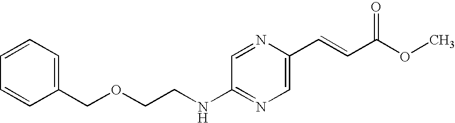 |
| 148 | 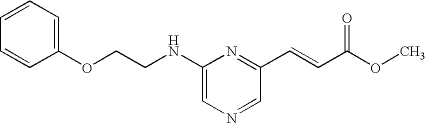 |
| 149 | 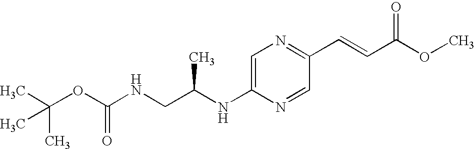 |
| 150 | 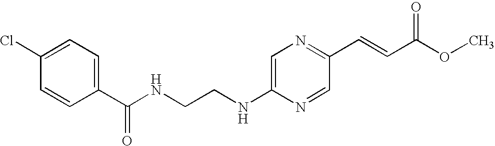 |
| 151 | 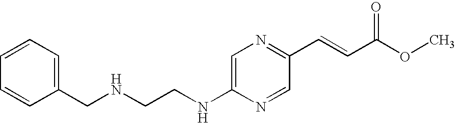 |
| 152 | 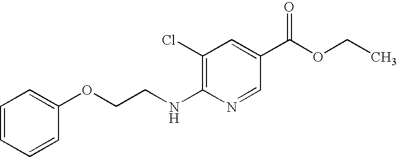 |
| 153 | 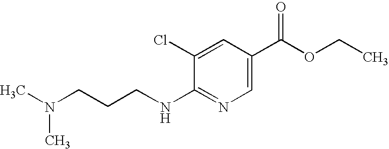 |
| 154 | 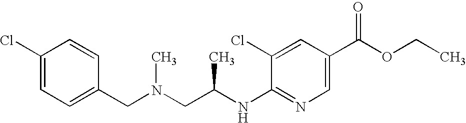 |
| 155 | 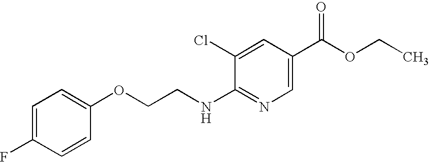 |
| 156 | 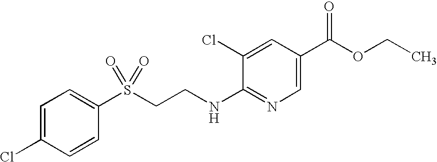 |
| 157 | 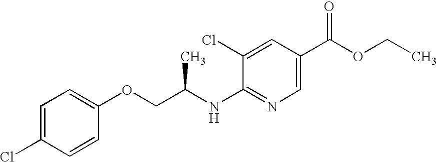 |
| 158 | 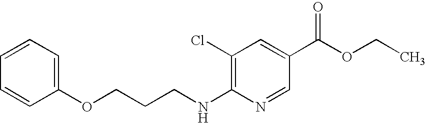 |
| 159 | 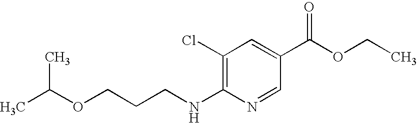 |
| 160 | 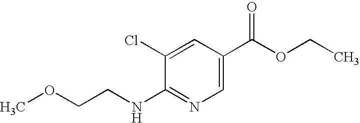 |
| 161 | 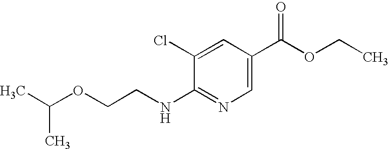 |
| 162 | 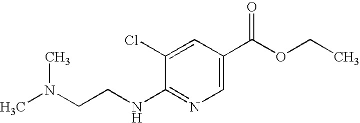 |
| 163 | 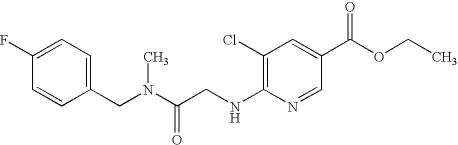 |
| 164 | 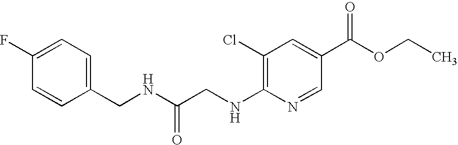 |
| 165 | 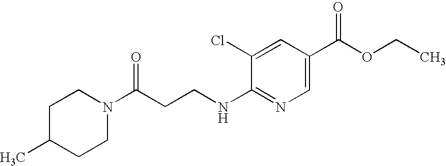 |
| 166 | 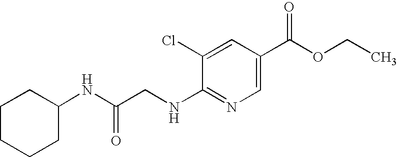 |
| 167 | 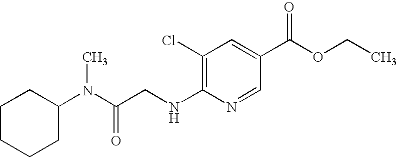 |
| 168 | 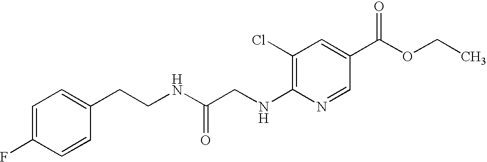 |
| 169 | 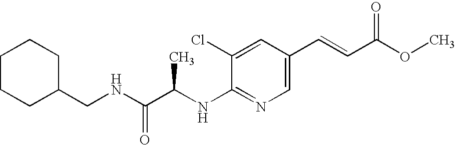 |
| 170 | 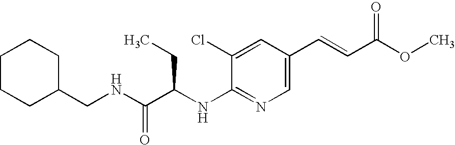 |
| 171 | 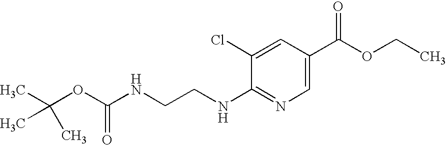 |
| 172 | 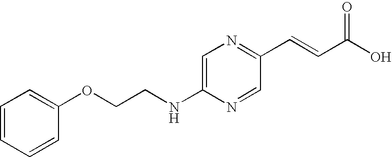 |
| 173 | 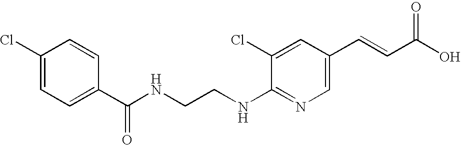 |
| 174 | 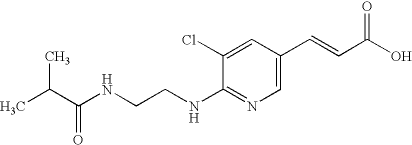 |
| 175 | 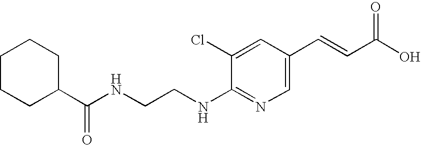 |
| 176 | 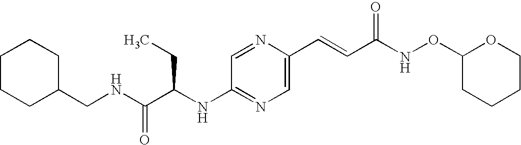 |
| 177 | 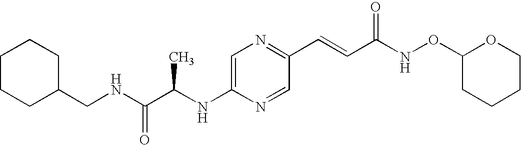 |
| 178 | 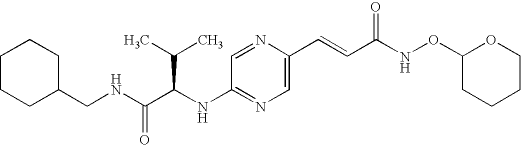 |
| 179 | 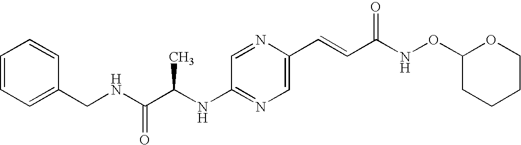 |
| 180 | 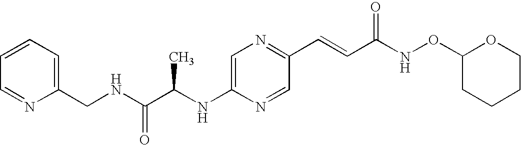 |
| 181 | 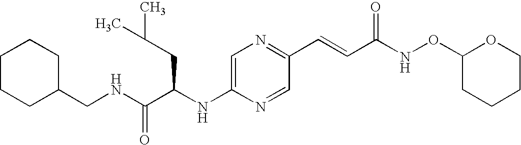 |
| 182 | 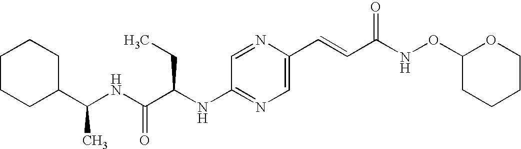 |
| 183 | 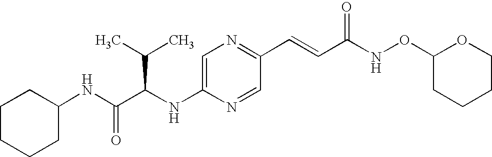 |
| 184 | 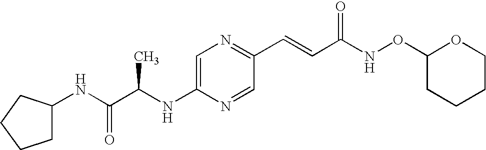 |
| 185 | 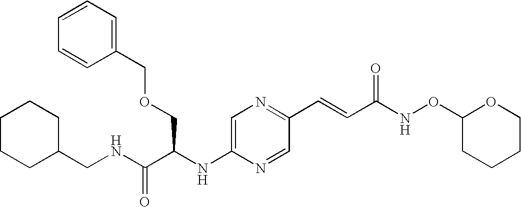 |
| 186 | 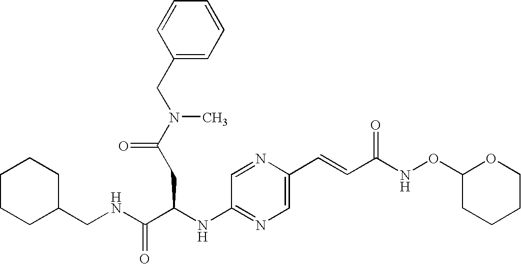 |
| 187 | 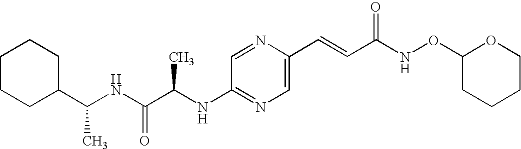 |
| 188 | 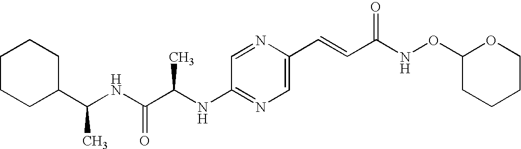 |
| 189 | 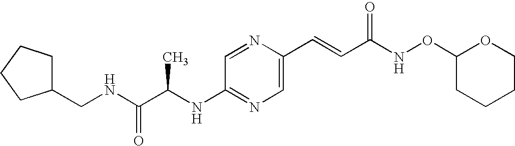 |
| 190 | 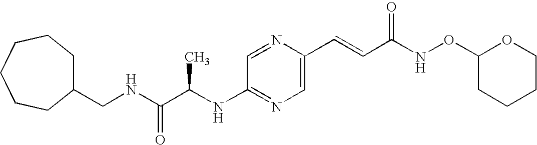 |
| 191 | 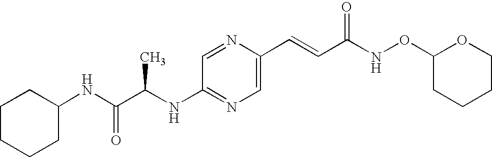 |
| 192 | 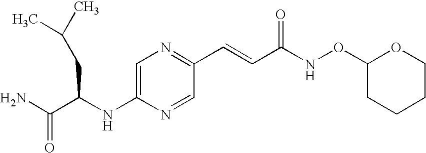 |
| 193 | 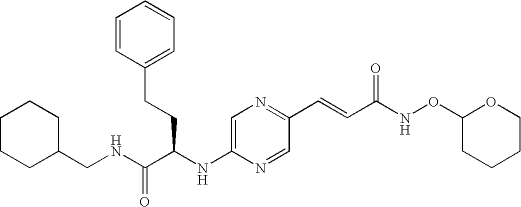 |
| 194 | 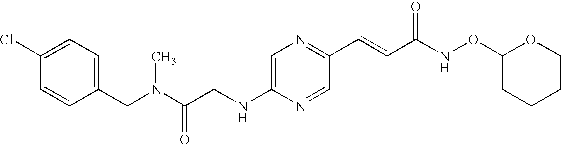 |
| 195 | 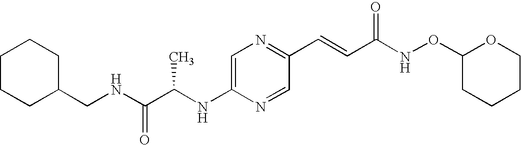 |
| 196 | 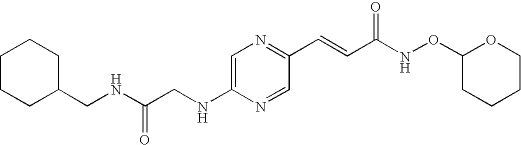 |
| 197 | 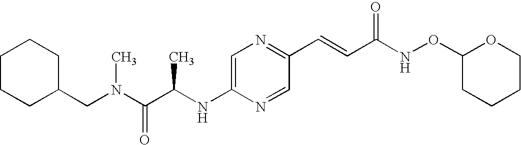 |
| 198 | 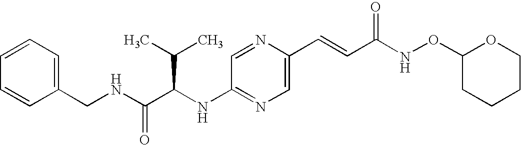 |
| 199 | 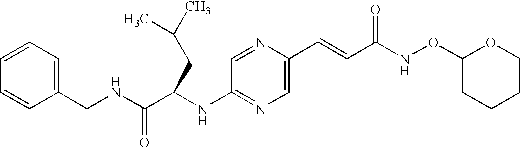 |
| 200 | 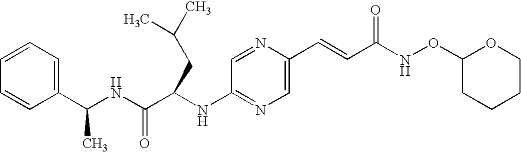 |
| 201 | 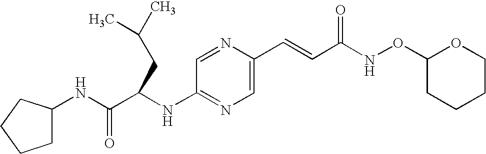 |
| 202 | 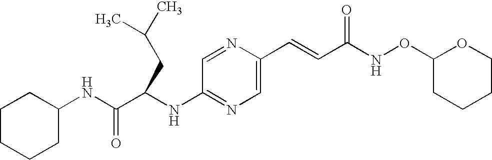 |
| 203 | 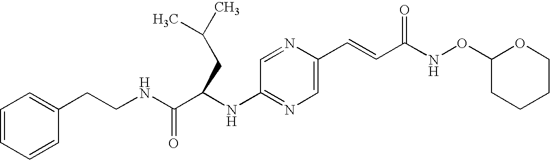 |
| 204 | 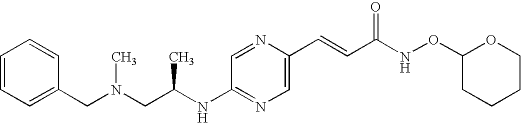 |
| 205 | 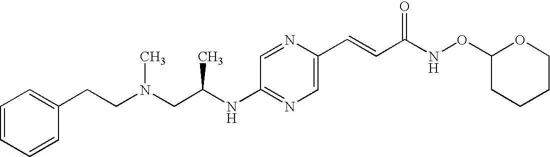 |
| 206 | 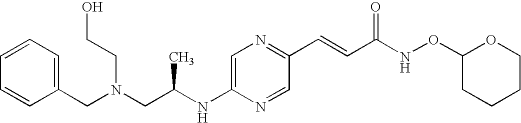 |
| 207 | 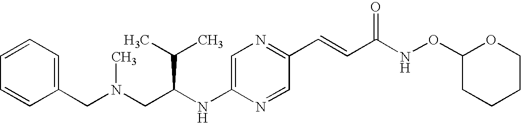 |
| 208 | 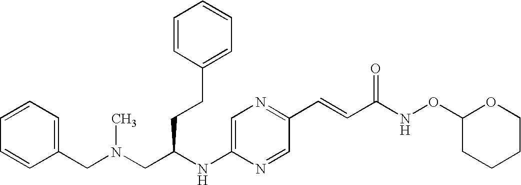 |
| 209 | 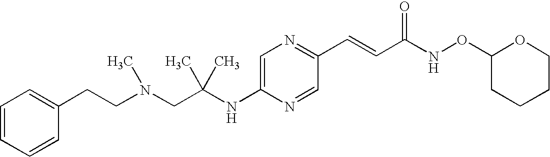 |
| 210 | 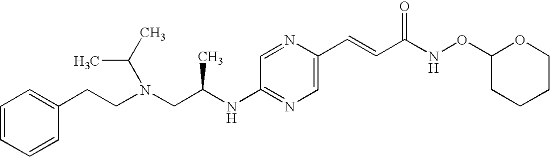 |
| 211 | 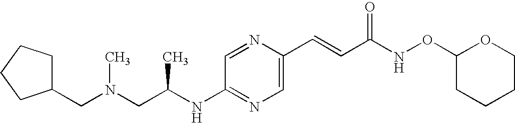 |
| 212 | 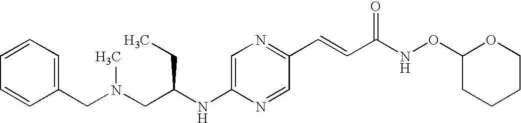 |
| 213 | 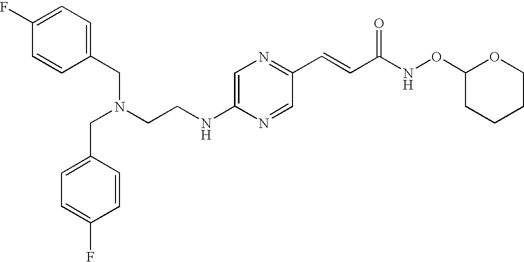 |
| 214 | 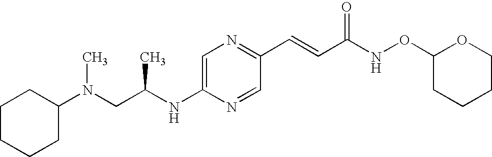 |
| 215 | 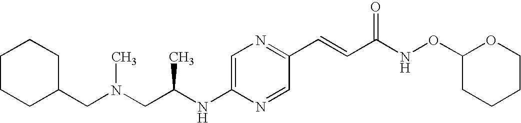 |
| 216 | 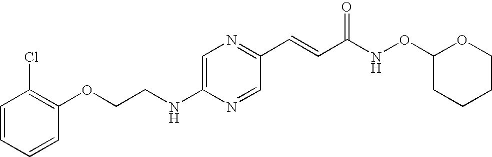 |
| 217 | 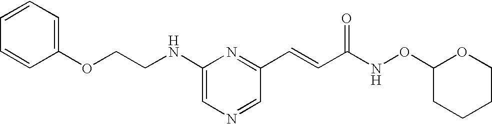 |
| 218 | 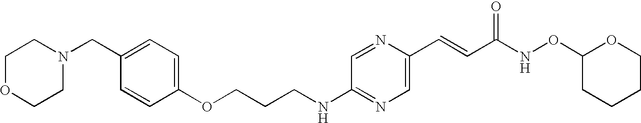 |
| 219 | 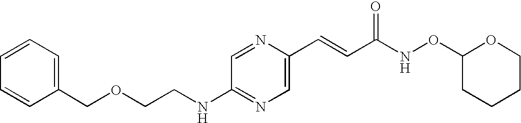 |
| 220 | 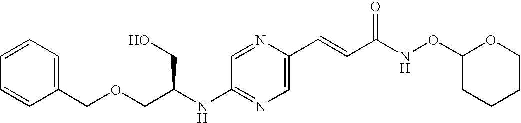 |
| 221 | 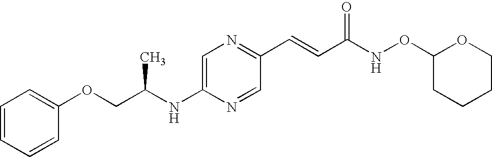 |
| 222 | 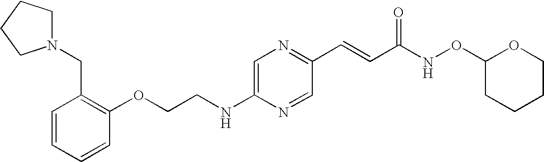 |
| 223 | 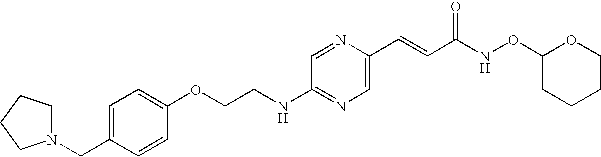 |
| 224 | 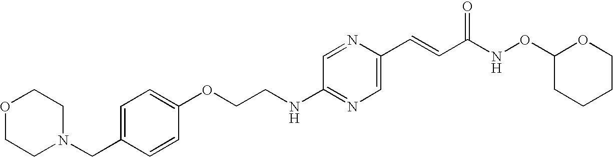 |
| 225 | 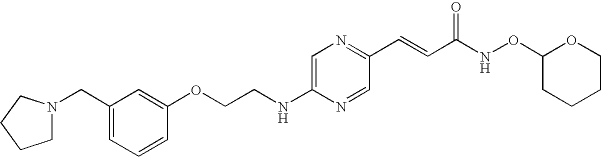 |
| 226 | 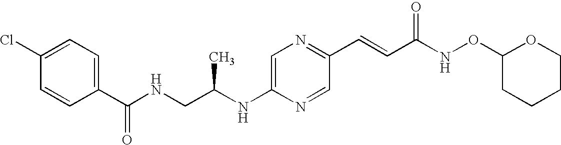 |
| 227 | 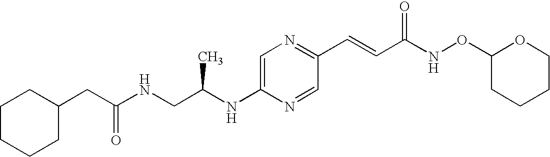 |
| 228 | 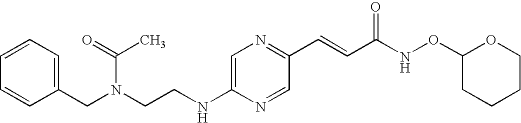 |
| 229 | 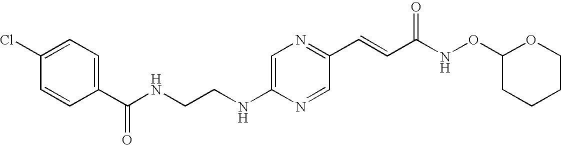 |
| 230 | 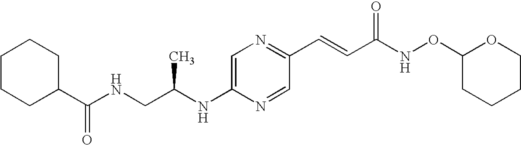 |
| 231 | 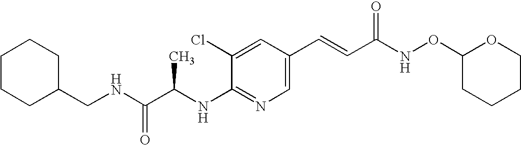 |
| 232 | 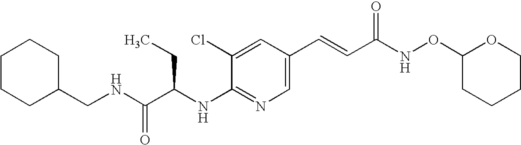 |
| 233 | 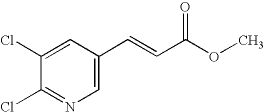 |
| 234 | 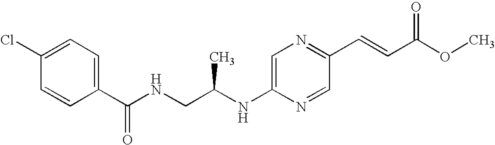 |
| 235 | 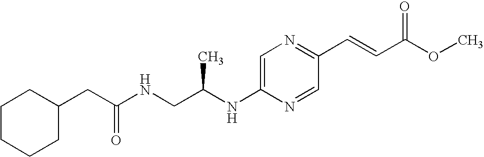 |
| 236 | 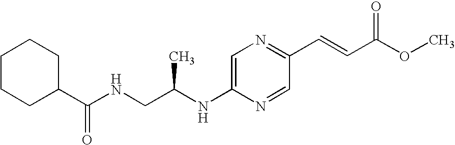 |
| 237 | 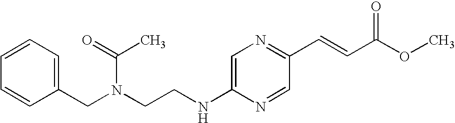 |
| 238 | 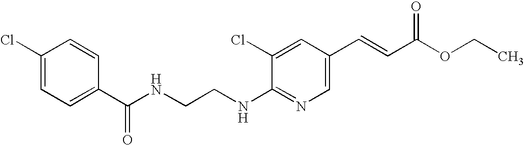 |
| 239 | 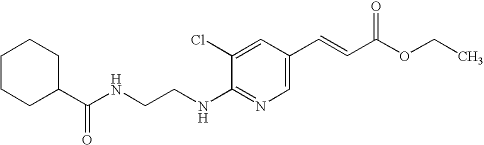 |
| 240 | 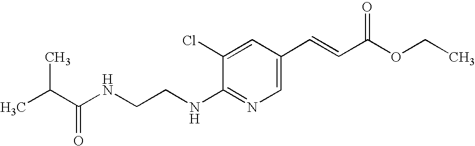 |
| 241 | 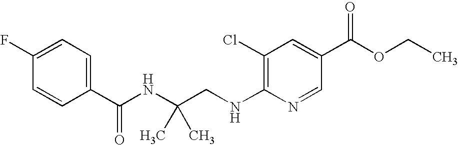 |
| 242 | 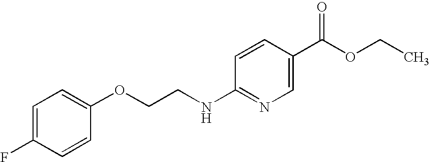 |
| 243 | 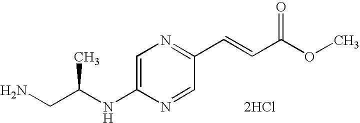 |
| 244 | 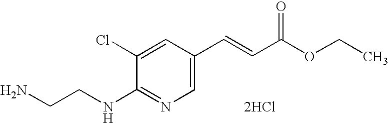 |
| 245 | 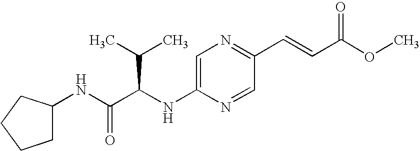 |
| 246 | 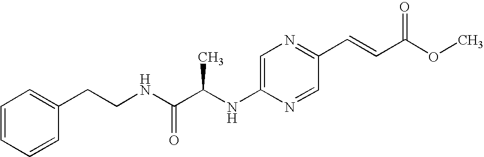 |
| 247 | 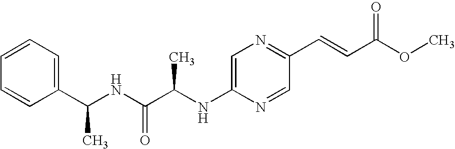 |
| 248 | 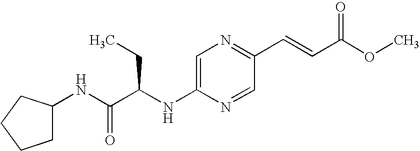 |
| 249 | 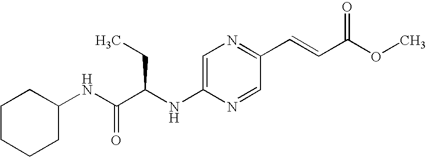 |
| 250 | 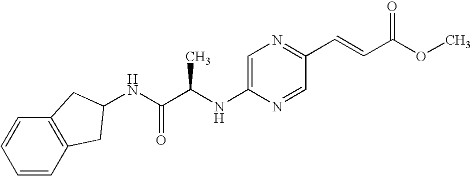 |
| 251 | 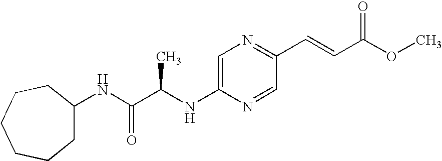 |
| 252 | 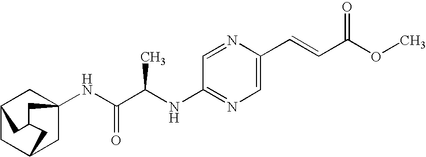 |
| 253 | 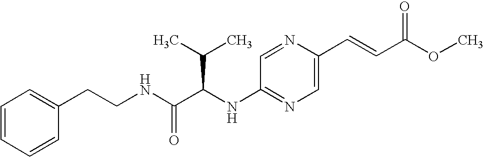 |
| 254 | 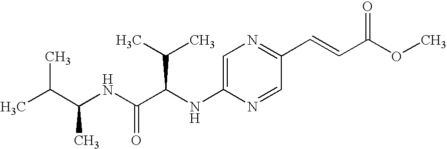 |
| 255 | 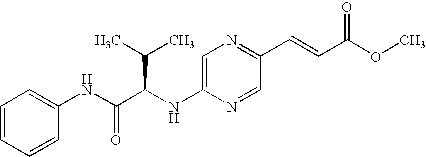 |
| 256 | 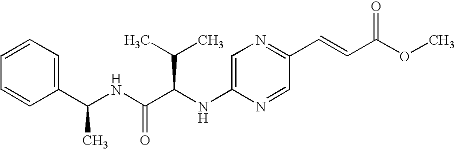 |
| 257 | 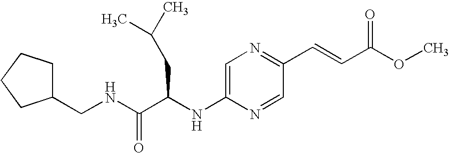 |
| 258 | 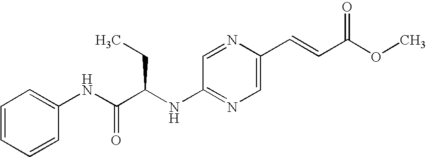 |
| 259 | 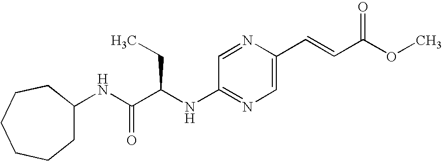 |
| 260 | 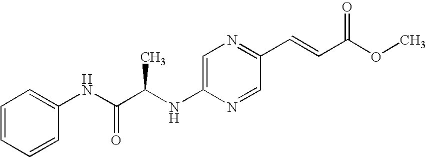 |
| 261 | 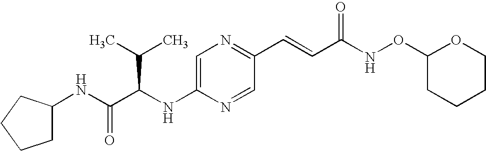 |
| 262 | 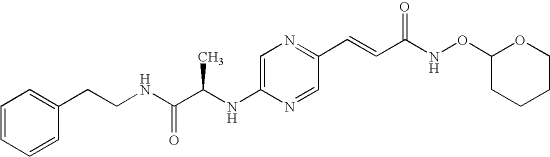 |
| 263 | 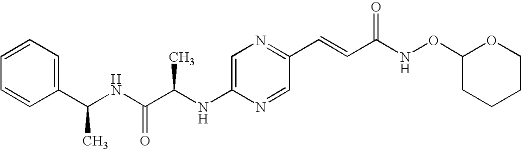 |
| 264 | 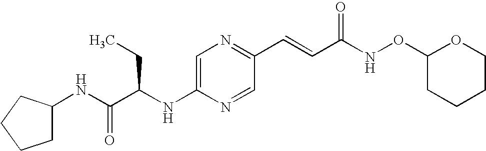 |
| 265 | 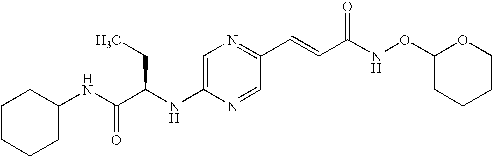 |
| 266 | 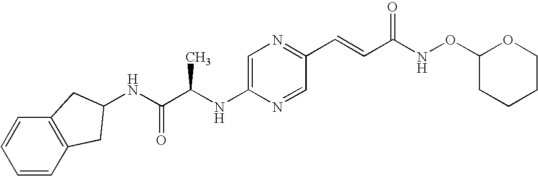 |
| 267 | 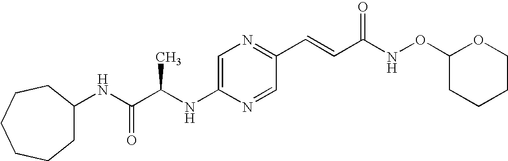 |
| 268 | 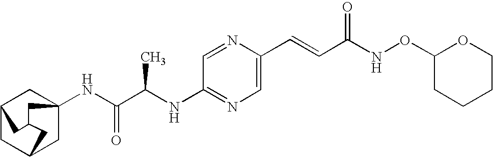 |
| 269 | 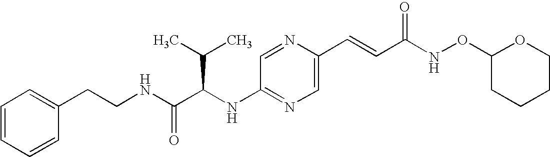 |
| 270 | 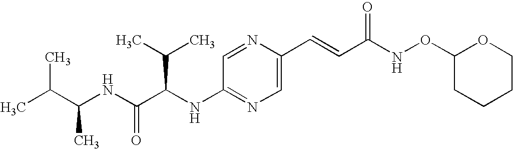 |
| 271 | 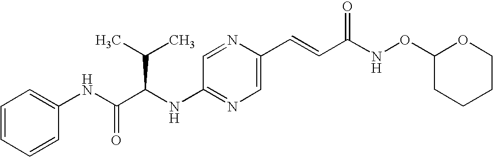 |
| 272 | 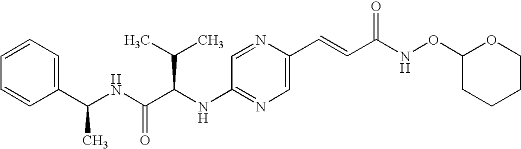 |
| 273 | 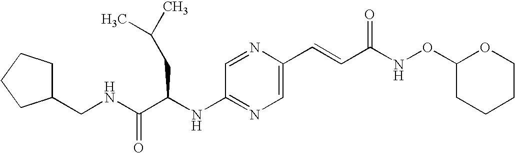 |
| 274 | 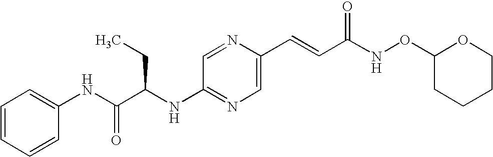 |
| 275 | 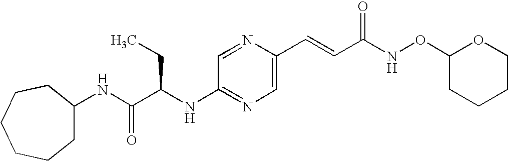 |
| 276 | 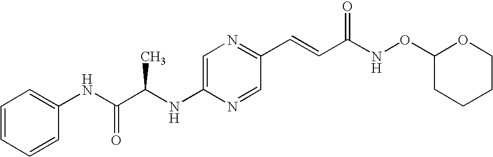 |
| 277 | 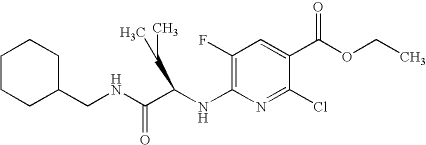 |
| 278 | 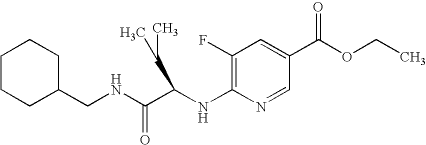 |
| 279 | 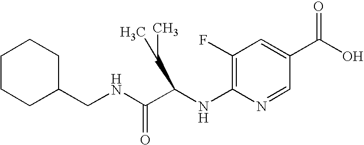 |
| 280 | 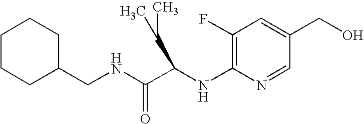 |
| 281 | 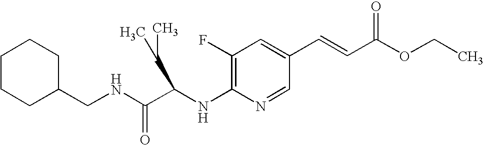 |
| 282 | 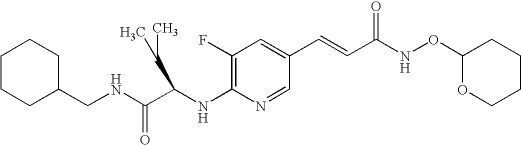 |

TABLE 3
Preparation number and analytical data

| Pr | Dat. |
|---|---|
| 1 | ESI-MS: 279 (M + H)+ |
| 2 | ESI-MS: 244 (M + H)+ |
| 3 | ESI-MS: 230 (M + H)+ |
| 4 | ESI-MS: 293 and 295 (M + H)+, 315 (M + Na)+ |
| 5 | ESI-MS: 327 and 329 (M + H)+ |
| 6 | ESI-MS: 361 and 363 (M + H)+, 383 and 385 (M + Na)+ |
| 7 | ESI-MS: 297 (M + H)+, 319 (M + Na)+ |
| 8 | ESI-MS: 354, 356 (M + H)+ |
| 9 | ESI-MS: 259 (M + H)+, 281 (M + Na)+ |
| 10 | 1H-NMR (DMSO-d6): δ 3.55-3.68 (2H, m), 4.06 (2H, t, |
| | J = 5.8 Hz), 4.30 (2H, d, J = 5.5 Hz), 4.92 (1H, t, J = 5.5 Hz), |
| | 6.52 (1H, d, J = 8.5 Hz), 6.93-7.02 (2H, m), 7.04-7.17 (2H, |
| | m), 7.35 (1H, dd, J = 2.3 Hz, 8.5 Hz), 7.92 (1H, d, J = 2.3 Hz) |
| 11 | 1H-NMR (DMSO-d6): δ 0.91 (3H, d, J = 6.2 Hz), 2.06-2.15 (5H, |
| | m), 2.91-2.99 (1H, m), 3.37-3.52 (2H, m), 7.21-7.34 (5H, m) |
| 12 | ESI-MS: 209 (M + H)+ |
| 13 | ESI-MS: 207 (M + H)+ |
| 14 | ESI-MS: 269 (M + H)+ |
| 15 | ESI-MS: 193 (M + H)+ |
| 16 | ESI-MS: 221 (M + H)+ |
| 17 | ESI-MS: 171 (M + H)+ |
| 18 | ESI-MS: 207 (M + H)+ |
| 19 | ESI-MS: 185 (M + H)+ |
| 20 | ESI-MS: 171 (M + H)+ |
| 21 | ESI-MS: 213 (M + H)+ |
| 22 | ESI-MS: 193 (M + H)+ |
| 23 | ESI-MS: 338 (M + H)+, 360 (M + Na)+ |
| 24 | ESI-MS: 324 (M + H)+ |
| 25 | ESI-MS: 312 (M + H)+ |
| 26 | ESI-MS: 338 (M + H)+, 360 (M + Na)+ |
| 27 | ESI-MS: 298 (M + H)+ |
| 28 | ESI-MS: 312 (M + H)+ |
| 29 | ESI-MS: 352 (M + H)+ |
| 30 | ESI-MS: 277 (M + H)+ |
| 31 | ESI-MS: 242 (M + H)+ |
| 32 | ESI-MS: 228 (M + H)+ |
| 33 | ESI-MS: 243 (M + H)+ |
| 34 | ESI-MS: 291 (M + H)+, 313 (M + Na)+ |
| 35 | ESI-MS: 381 and 383 (M + Na)+ |
| 36 | ESI-MS: 310 (M + H)+ |
| 37 | ESI-MS: 332 (M + Na)+ |
| 38 | ESI-MS: 352 and 354 (M + H)+ |
| 39 | ESI-MS: 336 (M + H)+, 358 (M + Na)+ |
| 40 | ESI-MS: 296 (M + H)+ |
| 41 | ESI-MS: 322 (M + H)+, 344 (M + Na)+ |
| 42 | ESI-MS: 358 (M + Na)+ |
| 43 | ESI-MS: 295 (M + H)+ |
| 44 | ESI-MS: 325 and 327 (M + H)+ |
| 45 | ESI-MS: 257 (M + H)+, 279 (M + Na)+ |
| 46 | ESI-MS: 215 (M + H)+ |
| 47 | ESI-MS: 261 (M + H)+, 283 (M + Na)+ |
| 48 | ESI-MS: 322 (M + Na)+ |
| 49 | ESI-MS: 350 (M + H)+ |
| 50 | ESI-MS: 239 (M + Na)+ |
| 51 | ESI-MS: 245 (M + H)+, 267 (M + Na)+ |
| 52 | 1H-NMR (DMSO-d6): δ 1.37 (9H, s), 3.07-3.19 (2H, m), |
| | 3.32-3.44 (2H, m), 3.32 (2H, d, J = 5.6 Hz), 5.05 (1H, t, |
| | J = 5.6 Hz), 6.40 (1H, t, J = 5.4 Hz), 6.92 (1H, t, J = 5.5 Hz), |
| | 7.52 (1H, d, J = 2.0 Hz), 7.90 (1H, d, J = 2.0 Hz) |
| 53 | ESI-MS: 407 (M + Na)+ |
| 54 | ESI-MS: 418 (M + H)+ |
| 55 | ESI-MS: 356 (M + H)+, 378 (M + Na)+ |
| 56 | ESI-MS: 522 and 524 (M + Na)+ |
| 57 | ESI-MS: 432 (M + H)+, 454 (M + Na)+ |
| 58 | ESI-MS: 477 (M + H)+, 499 (M + Na)+ |
| 59 | ESI-MS: 466 and 468 (M + H)+, 488 and 490 (M + Na)+ |
| 60 | ESI-MS: 437 (M + H)+ |
| 61 | ESI-MS: 451 (M + H)+ |
| 62 | ESI-MS: 436 (M + H)+, 458 (M + Na)+ |
| 63 | ESI-MS: 383 (M + H)+ |
| 64 | ESI-MS: 451 (M + H)+ |
| 65 | ESI-MS: 477 (M + H)+, 499 (M + Na)+ |
| 66 | ESI-MS: 493 and 495 (M + H)+ |
| 67 | ESI-MS: 463 (M + H)+, 485 (M + Na)+ |
| 68 | ESI-MS: 398 (M + H)+, 420 (M + Na)+ |
| 69 | ESI-MS: 384 (M + H)+, 406 (M + Na)+ |
| 70 | ESI-MS: 369 (M + H)+ |
| 71 | ESI-MS: 402 (M + H)+, 424 (M + Na)+ |
| 72 | ESI-MS: 479 and 482 (M + H)+, 501 and 503 (M + Na)+ |
| 73 | ESI-MS: 411 (M + H)+ |
| 74 | ESI-MS: 451 (M + H)+, 473 (M + Na)+ |
| 75 | ESI-MS: 491 (M + H)+, 513 (M + Na)+ |
| 76 | ESI-MS: 235 and 237 (M + H)+, 257 and 259 (M + Na)+ |
| 77 | ESI-MS: 392 (M + Na)+ |
| 78 | ESI-MS: 317 (M − H)− |
| 79 | ESI-MS: 270 (M + H)+ |
| 80 | ESI-MS: 394 and 396 (M + H)+ |
| 81 | ESI-MS: 444 (M + Na)+ |
| 82 | ESI-MS: 335 (M − H)− |
| 83 | ESI-MS: 376 (M − H)− |
| 84 | ESI-MS: 336 (M − H)− |
| 85 | ESI-MS: 297 (M − H)− |
| 86 | ESI-MS: 376 (M − H)− |
| 87 | ESI-MS: 362 (M − H)− |
| 88 | ESI-MS: 350 (M − H)− |
| 89 | ESI-MS: 365 and 367 (M − H)− |
| 90 | ESI-MS: 331 (M − H)− |
| 91 | ESI-MS: 284 (M + H)+ |
| 92 | ESI-MS: 283 (M − H)− |
| 93 | ESI-MS: 255 (M − H)− |
| 94 | ESI-MS: 350 (M − H)− |
| 95 | ESI-MS: 301 (M − H)− |
| 96 | ESI-MS: 390 (M − H)− |
| 97 | ESI-MS: 361 (M + H)+, 383 (M + Na)+ |
| 98 | ESI-MS: 375 (M + H)+, 397 (M + Na)+ |
| 99 | ESI-MS: 459 (M + Na)+ |
| 100 | ESI-MS: 342 (M + H)+, 364 (M + Na)+, 705 (2M + Na)+ |
| 101 | ESI-MS: 453 (M + H)+, 475 (M + Na)+ |
| 102 | ESI-MS: 389 (M + H)+, 411 (M + Na)+ |
| 103 | ESI-MS: 293 (M + H)+, 315 (M + Na)+ |
| 104 | ESI-MS: 383 (M + Na)+, 743 (2M + Na)+ |
| 105 | ESI-MS: 383 (M + Na)+, 743 (2M + Na)+ |
| 106 | ESI-MS: 494 (M + H)+, 516 (M + Na)+ |
| 107 | ESI-MS: 383 (M + Na)+, 743 (2M + Na)+ |
| 108 | ESI-MS: 355 (M + Na)+, 687 (2M + Na)+ |
| 109 | ESI-MS: 355 (M + Na)+, 687 (2M + Na)+ |
| 110 | ESI-MS: 375 (M + H)+, 397 (M + Na)+ |
| 111 | ESI-MS: 319 (M + H)+, 341 (M + Na)+ |
| 112 | ESI-MS: 361 (M + H)+, 383 (M + Na)+ |
| 113 | ESI-MS: 341 (M + H)+, 363 (M + Na)+ |
| 114 | ESI-MS: 375 (M + H)+, 397 (M + Na)+, 771 (2M + Na)+ |
| 115 | ESI-MS: 347 (M + H)+, 369 (M + Na)+ |
| 116 | ESI-MS: 383 (M + Na)+ |
| 117 | ESI-MS: 333 (M + H)+, 355 (M + Na)+ |
| 118 | ESI-MS: 347 (M + H)+, 369 (M + Na)+, 715 (2M + Na)+ |
| 119 | ESI-MS: 391 (M + Na)+ |
| 120 | ESI-MS: 405 (M + Na)+ |
| 121 | ESI-MS: 397 (M + Na)+ |
| 122 | ESI-MS: 419 (M + Na)+ |
| 123 | ESI-MS: 383 (M + Na)+ |
| 124 | ESI-MS: 419 (M + Na)+ |
| 125 | ESI-MS: 272 (M + H)+ |
| 126 | ESI-MS: 341 (M + H)+ |
| 127 | ESI-MS: 371 (M + H)+, 393 (M + Na)+ |
| 128 | ESI-MS: 369 (M + H)+, 391 (M + Na)+ |
| 129 | ESI-MS: 431 (M + H)+ |
| 130 | ESI-MS: 355 (M + H)+ |
| 131 | ESI-MS: 383 (M + H)+ |
| 132 | ESI-MS: 333 (M + H)+ |
| 133 | ESI-MS: 347 (M + H)+ |
| 134 | ESI-MS: 333 (M + H)+ |
| 135 | ESI-MS: 439 (M + H)+ |
| 136 | ESI-MS: 369 (M + H)+ |
| 137 | ESI-MS: 355 (M + H)+ |
| 138 | ESI-MS: 334 (M + H)+, 356 (M + Na)+ |
| 139 | ESI-MS: 399 (M + H)+ |
| 140 | ESI-MS: 383 (M + H)+ |
| 141 | ESI-MS: 314 (M + H)+, 336 (M + Na)+ |
| 142 | ESI-MS: 383 (M + H)+ |
| 143 | ESI-MS: 383 (M + H)+ |
| 144 | ESI-MS: 411 (M − H)− |
| 145 | ESI-MS: 300 (M + H)+, 322 (M + Na)+ |
| 146 | ESI-MS: 344 (M + H)+, 366 (M + Na)+ |
| 147 | ESI-MS: 314 (M + H)+, 336 (M + Na)+ |
| 148 | ESI-MS: 298 (M − H)− |
| 149 | ESI-MS: 359 (M + Na)+, 695 (2M + Na)+ |
| 150 | ESI-MS: 361 (M + H)+, 383 (M + Na)+ |
| 151 | ESI-MS: 313 (M + H)+ |
| 152 | ESI-MS: 321 (M + H)+ |
| 153 | ESI-MS: 286 (M + H)+ |
| 154 | ESI-MS: 396 and 398 (M + H)+ |
| 155 | ESI-MS: 339 (M + H)+ |
| 156 | 1H-NMR (DMSO-d6): δ 1.30 (3H, t, J = 7.1 Hz), 3.66-3.84 (4H, |
| | m), 4.27 (2H, q, J = 7.1 Hz), 7.24-7.32 (1H, m), |
| | 7.62-7.76 (2H, m), 7.84-7.95 (3H, m), 8.48 (1H, d, J = 2.0 Hz) |
| 157 | ESI-MS: 369 and 371 (M + H)+ |
| 158 | ESI-MS: 335 (M + H)+, 367 (M + Na)+ |
| 159 | ESI-MS: 323 (M + Na)+ |
| 160 | ESI-MS: 259 (M + H)+, 281 (M + Na)+ |
| 161 | ESI-MS: 287 (M + H)+, 309 (M + Na)+ |
| 162 | ESI-MS: 272 (M + H)+ |
| 163 | ESI-MS: 380 (M + H)+, 402 (M + Na)+ |
| 164 | ESI-MS: 366 (M + H)+, 388 (M + Na)+ |
| 165 | ESI-MS: 354 (M + H)+, 376 (M + Na)+ |
| 166 | ESI-MS: 340 (M + H)+, 362 (M + Na)+ |
| 167 | ESI-MS: 354 (M + H)+ |
| 168 | ESI-MS: 380 (M + H)+ |
| 169 | ESI-MS: 380 (M + H)+, 402 (M + Na)+ |
| 170 | ESI-MS: 394 (M + H)+, 416 (M + Na)+ |
| 171 | ESI-MS: 366 (M + Na)+ |
| 172 | ESI-MS: 284 (M − H)− |
| 173 | ESI-MS: 380 and 382 (M + H)+ |
| 174 | ESI-MS: 312 (M + H)+ |
| 175 | ESI-MS: 350 (M − H)− |
| 176 | ESI-MS: 468 (M + Na)+ |
| 177 | ESI-MS: 454 (M + Na)+ |
| 178 | ESI-MS: 482 (M + Na)+ |
| 179 | ESI-MS: 448 (M + Na)+ |
| 180 | ESI-MS: 449 (M + Na)+ |
| 181 | ESI-MS: 496 (M + Na)+ |
| 182 | ESI-MS: 482 (M + Na)+ |
| 183 | ESI-MS: 468 (M + Na)+ |
| 184 | ESI-MS: 426 (M + Na)+ |
| 185 | ESI-MS: 560 (M + Na)+ |
| 186 | ESI-MS: 601 (M + Na)+ |
| 187 | ESI-MS: 468 (M + Na)+ |
| 188 | ESI-MS: 468 (M + Na)+ |
| 189 | ESI-MS: 440 (M + Na)+ |
| 190 | ESI-MS: 468 (M + Na)+ |
| 191 | ESI-MS: 440 (M + Na)+ |
| 192 | ESI-MS: 400 (M + Na)+ |
| 193 | ESI-MS: 522 (M + H)+, 544 (M + Na)+ |
| 194 | ESI-MS: 460 (M + H)+ |
| 195 | ESI-MS: 432 (M + H)+, 454 (M + Na)+, 885 (2M + Na)+ |
| 196 | ESI-MS: 418 (M + H)+, 440 (M + Na)+, 857 (2M + Na)+ |
| 197 | ESI-MS: 446 (M + H)+, 468 (M + Na)+ |
| 198 | ESI-MS: 476 (M + Na)+ |
| 199 | ESI-MS: 490 (M + Na)+ |
| 200 | ESI-MS: 504 (M + Na)+ |
| 201 | ESI-MS: 468 (M + Na)+ |
| 202 | ESI-MS: 482 (M + Na)+ |
| 203 | ESI-MS: 504 (M + Na)+ |
| 204 | ESI-MS: 426 (M + H)+ |
| 205 | ESI-MS: 440 (M + H)+ |
| 206 | ESI-MS: 456 (M + H)+, 478 (M + Na)+ |
| 207 | ESI-MS: 454 (M + H)+, 476 (M + Na)+ |
| 208 | ESI-MS: 516 (M + H)+ |
| 209 | ESI-MS: 454 (M + H)+ |
| 210 | ESI-MS: 468 (M + H)+ |
| 211 | ESI-MS: 418 (M + H)+, 440 (M + Na)+ |
| 212 | ESI-MS: 440 (M + H)+ |
| 213 | ESI-MS: 524 (M + H)+ |
| 214 | ESI-MS: 418 (M + H)+ |
| 215 | ESI-MS: 432 (M + H)+ |
| 216 | ESI-MS: 417 (M − H)− |
| 217 | ESI-MS: 385 (M + H)+, 407 (M + Na)+ |
| 218 | ESI-MS: 498 (M + H)+, 520 (M + Na)+ |
| 219 | ESI-MS: 421 (M + Na)+ |
| 220 | ESI-MS: 451 (M + Na)+ |
| 221 | ESI-MS: 399 (M + H)+, 421 (M + Na)+ |
| 222 | ESI-MS: 468 (M + H)+ |
| 223 | ESI-MS: 468 (M + H)+ |
| 224 | ESI-MS: 484 (M + H)+ |
| 225 | ESI-MS: 468 (M + H)+ |
| 226 | ESI-MS: 482 (M + Na)+ |
| 227 | ESI-MS: 468 (M + Na)+ |
| 228 | ESI-MS: 440 (M + H)+, 462 (M + Na)+ |
| 229 | ESI-MS: 446 (M + H)+, 468 (M + Na)+ |
| 230 | ESI-MS: 454 (M + Na)+ |
| 231 | ESI-MS: 465 (M + H)+, 487 (M + Na)+ |
| 232 | ESI-MS: 501 and 503 (M + Na)+ |
| 233 | 1H-NMR (DMSO-d6): δ 3.75 (3H, s), 6.94 (1H, d, J = 16.2 Hz), |
| | 7.68 (1H, d, J = 16.2 Hz), 8.61 (1H, d, J = 2.1 Hz), 8.74 (1H, |
| | d, J = 2.1 Hz) |
| 234 | ESI-MS: 375 (M + H)+, 397 (M + Na)+ |
| 235 | ESI-MS: 361 (M + H)+, 383 (M + Na)+, 743 (2M + Na)+ |
| 236 | ESI-MS: 347 (M + H)+, 369 (M + Na)+ |
| 237 | ESI-MS: 355 (M + H)+, 377 (M + Na)+ |
| 238 | ESI-MS: 408 and 410 (M + H)+, 430 and 432 (M + Na)+ |
| 239 | ESI-MS: 380 (M + H)+, 402 (M + Na)+ |
| 240 | ESI-MS: 340 (M + H)+ |
| 241 | ESI-MS: 394 (M + H)+ |
| 242 | ESI-MS: 305 (M + H)+, 327 (M + Na)+ |
| 243 | ESI-MS: 237 (M + H)+ |
| 244 | ESI-MS: 270 (M + H)+ |
| 245 | 1H-NMR (DMSO-d6): δ 0.90 (3H, d, J = 6.8 Hz), 0.91 (3H, d, |
| | J = 6.8 Hz), 1.32-1.69 (6H, m), 1.71-1.81 (2H, m), |
| | 1.99-2.08 (1H, m), 3.70 (3H, s), 3.95-4.05 (1H, m), |
| | 4.34-4.39 (1H, m), 6.53 (1H, d, J = 15.5 Hz), 7.57 (1H, d, |
| | J = 15.5 Hz), 7.76 (1H, d, J = 8.7 Hz), 8.01 (1H, d, J = 7.2 Hz), |
| | 8.18 (1H, s), 8.19 (1H, s) |
| 246 | ESI-MS: 377 (M + Na)+ |
| 247 | ESI-MS: 355 (M + H)+, 377 (M + Na)+ |
| 248 | ESI-MS: 333 (M + H)+, 355 (M + Na)+ |
| 249 | ESI-MS: 347 (M + H)+, 369 (M + Na)+ |
| 250 | ESI-MS: 367 (M + H)+, 389 (M + Na)+ |
| 251 | ESI-MS: 347 (M + H)+, 369 (M + Na)+ |
| 252 | ESI-MS: 385 (M + H)+, 407 (M + Na)+ |
| 253 | ESI-MS: 405 (M + Na)+ |
| 254 | ESI-MS: 349 (M + H)+, 371 (M + Na)+ |
| 255 | ESI-MS: 355 (M + H)+, 377 (M + Na)+ |
| 256 | ESI-MS: 405 (M + Na)+ |
| 257 | ESI-MS: 397 (M + Na)+ |
| 258 | ESI-MS: 341 (M + H)+, 363 (M + Na)+ |
| 259 | ESI-MS: 361 (M + H)+, 383 (M + Na)+ |
| 260 | ESI-MS: 327 (M + H)+, 349 (M + Na)+ |
| 261 | ESI-MS: 454 (M + Na)+ |
| 262 | ESI-MS: 462 (M + Na)+ |
| 263 | ESI-MS: 440 (M + H)+, 462 (M + Na)+ |
| 264 | ESI-MS: 440 (M + Na)+ |
| 265 | ESI-MS: 454 (M + Na)+ |
| 266 | ESI-MS: 474 (M + Na)+ |
| 267 | ESI-MS: 454 (M + Na)+ |
| 268 | ESI-MS: 468 (M − H)− |
| 269 | ESI-MS: 490 (M + Na)+ |
| 270 | ESI-MS: 434 (M + H)+, 456 (M + Na)+ |
| 271 | ESI-MS: 440 (M + H)+, 462 (M + Na)+ |
| 272 | ESI-MS: 490 (M + H)+ |
| 273 | ESI-MS: 460 (M + H)+ |
| 274 | ESI-MS: 448 (M + Na)+ |
| 275 | ESI-MS: 468 (M + Na)+ |
| 276 | ESI-MS: 434 (M + Na)+ |
| 277 | ESI-MS: 414 (M + H)+ |
| 278 | ESI-MS: 380 (M + H)+ |
| 279 | ESI-MS: 374 (M + Na)+ |
| 280 | ESI-MS: 338 (M + H)+ |
| 281 | ESI-MS: 406 (M + H)+ |
| 282 | ESI-MS: 499 (M + Na)+ |

Pr: Preparation number;
Dat.: analytical data;

### EXAMPLE 1

2M HCl in EtOH (2.5 mL) was added to the solution of (2E)-3-{5-chloro-6-[(2-phenoxyethyl)amino]-3-pyridinyl}-N-(tetrahydro-2H-pyran-2-yloxy)acrylamide (0.70 g) in EtOH (14 ml) and the mixture was stirred at ambient temperature for 2 hours. To the reaction mixture was added AcOEt and isolated precipitate was collected by filtration to give (2E)-3-{5-chloro-6-[(2-phenoxyethyl)amino]-3-pyridinyl}-N-hydroxyacrylamide hydrochloride (0.54 g).

The compounds disclosed in Examples 2, 5, 6, 7, 8, 9, 11, 12, 13, 14, 15, 16, 17, 18, 19, 20, 21, 22, 23, 25, 26, 27, 28, 29, 30, 31, 32, 34, 35, 36, 37, 38, 39, 40, 45, 46, 47, 48, 52 and 53 were obtained in a similar manner to that of Example 1.

### EXAMPLE 3

2M HCl in EtOH (1.4 mL) was added to the solution of 4-chloro-N-{2-[(3-chloro-5-{(1E)-3-oxo-3-[(tetrahydro-2H-pyran-2-yloxy)amino]-1-propen-1-yl}-2-pyridinyl)amino]ethyl}benzamide (0.45 g) in EtOH (18 ml) and the mixture was stirred at ambient temperature for 2 hours. The solvent was removed by concentration and the The residue was triturated with a mixture of EtOH, THF and AcOEt to give 4-chloro-N-[2-({3-chloro-5-[(1E)-3-(hydroxyamino)-3-oxo-1-propen-1-yl]-2-pyridinyl}amino)ethyl]benzamide hydrochloride (0.32 g).

The compounds disclosed in Examples 4 and 10 were obtained in a similar manner to that of Example 3.

### EXAMPLE 24

2M HCl in EtOH (1.6 mL) was added to the solution of. N¹-(cyclohexylmethyl)-N²-(5-{(1E)-3-oxo-3-[(tetrahydro-2H-pyran-2-yloxy)amino]prop-1-en-1-yl}pyrazin-2-yl)-D-alaninamide (0.46 g) in MeOH (6.9 ml) and the mixture was stirred at ambient temperature for 2.5 hours. To the reaction mixture was added a solution of AcOEt and IPE and isolated precipitate was collected by filtration to give N¹-(cyclohexylmethyl)-N²-{5-[(1E)-3-(hydroxyamino)-3-oxopropl-en-1-yl]pyrazin-2-yl}-D-alaninamide hydrochloride (0.17 g).

The compounds disclosed in Examples 42, 43, 60, 64, 65, 71, 74 and 96 were obtained in a similar manner to that of Example 24.

### EXAMPLE 33

2M HCl in EtOH (1.4 mL) was added to the solution of 4-chloro-N-{(2R)-2-[(5-{(1E)-3-oxo-3-[(tetrahydro-2H-pyran-2-yloxy)amino]prop-1-en-1-yl}pyrazin-2-yl)amino]propyl}benzamide (0.42 g) in EtOH (8.4 ml) and the mixture was stirred at ambient temperature for 3.5 hours. To the reaction mixture was added a solution of AcOEt and ether and isolated precipitate was collected by filtration to give 4-chloro-N-[(2R)-2-({5-[(1E)-3-(hydroxyamino)-3-oxoprop-1-en-1-yl]pyrazin-2-yl}amino)propyl]benzamide hydrochloride (0.31 g).

The compounds disclosed in Examples 41, 44 and 66 were obtained in a similar manner to that of Example 33.

### EXAMPLE 49

2M HCl in EtOH (1.3 mL) was added to the solution N²-(3-chloro-5-{(1E)-3-oxo-3-[(tetrahydro-2H-pyran-2-yloxy)amino]prop-1-en-1-yl}pyridin-2-yl)-N¹-(cyclohexylmethyl)-D-alaninamide (0.3 g) in EtOH (3.0 ml) and the mixture was stirred at ambient temperature for 3 hours. The solvent was removed by concentration and the residue was added a mixture of AcOEt and water. The mixture was adjusted to pH 7 with saturated sodium hydrogen carbonate aq. The separated organic layer was washed with water, dried over magnesium sulfate and evaporated in vacuo. The residue was triturated with IPE to give N²-{3-chloro-5-[(1E)-3-(hydroxyamino)-3-oxoprop-1-en-1-yl]pyridin-2-yl}-N¹-(cyclohexylmethyl)-D-alaninamide (95 mg).

The compound disclosed in Example 56 was obtained in a similar manner to that of Example 49.

### EXAMPLE 50

2M HCl in EtOH (3.1 mL) was added to the solution of (2E)-3-(5-{[(1R)-1-{[benzyl(methyl)amino]methyl}-3-phenylpropyl]amino}pyrazin-2-yl)-N-(tetrahydro-2H-pyran-2-yloxy)acrylamide (0.8 g) in EtOH (4 ml) and the mixture was stirred at ambient temperature for 3 hours. To the reaction mixture was added AcOEt and isolated precipitate was collected by filtration. The precipitate was added a mixture of AcOEt, THF and water. The mixture was adjusted to pH 8 with saturated sodium hydrogen carbonate aq. The separated organic layer was dried over magnesium sulfate and evaporated in vacuo. The residue was triturated with IPE to give (2E)-3-(5-{[(1R)-1-{[benzyl(methyl)amino]methyl}-3-phenylpropyl]amino}pyrazin-2-yl)-N-hydroxyacrylamide (0.15 g).

### EXAMPLE 51

2M HCl in EtOH (2.7 mL) was added to the solution of (2R)-N-(cyclohexylmethyl)-2-[(5-{(1E)-3-oxo-3-[(tetrahydro-2H-pyran-2-yloxy)amino]prop-1-en-1-yl)pyrazin-2-yl)amino]butanamide (0.8 g) in EtOH (16 ml) and the mixture was stirred at ambient temperature for 2.5 hours. The solvent was removed by concentration and the residue was added a mixture of AcOEt and water. The mixture was adjusted to pH 5 with saturated sodium hydrogen carbonate aq. The separated organic layer was dried over magnesium sulfate and evaporated in vacuo. The residue was triturated with ether to give (2R)-N-(cyclohexylmethyl)-2-({5-[(1E)-3-(hydroxyamino)-3-oxoprop-1-en-1-yl]pyrazin-2-yl}amino)butanamide (0.45 g).

The compounds disclosed in Examples 54, 55, 57, 58, 59, 61, 62, 63, 68, 69, 70, 72, 73, 75, 76, 77, 78, 79, 80, 81, 82, 83, 84, 85, 86, 87, 88, 89, 90, 91, 92, 93, 94, and 95 were obtained in a similar manner to that of Example 51.

### EXAMPLE 67

2M HCl in EtOH (1.0 mL) was added to the solution of (2E)-3-[5-({(1R)-2-[(cyclohexylacetyl)amino]-1-methylethyl}amino)pyrazin-2-yl]-N-(tetrahydro-2H-pyran-2-yloxy)acrylamide (0.3 g) in EtOH (6.0 ml) and the mixture was stirred at ambient temperature for 2.5 hours. The solvent was removed by concentration and the residue was added a mixture of AcOEt and water. The mixture was adjusted to pH 6 with saturated sodium hydrogen carbonate aq. and isolated precipitate was collected by filtration to give (2E)-3-[5-({(1R)-2-[(cyclohexylacetyl)amino]-1-methylethyl}amino)pyrazin-2-yl]N-hydroxyacrylamide (0.21 g).

### EXAMPLE 97

To a solution of N¹-(cyclohexylmethyl)-N²-(3-fluoro-5-{(1E)-3-oxo-3-[(tetrahydro-2H-pyran-2-yloxy)amino]prop-1-en-1-yl}pyridin-2-yl)-D-valinamide (220 mg) in EtOH (3.3 mL) was added 2M HCl in EtOH (0.92 mL) at ambient temperature. The reaction mixture was stirred at ambient temperature for 2 hours, and evaporated under reduced pressure. A mixture of water and AcOEt was added to the residue, the pH of the aqueous layer was adjusted to ca.7 with aqueous sodium hydrogen carbonate. The separated organic layer was washed with brine, dried over anhydrous magnesium sulfate, filtered and evaporated under reduced pressure. The resulting residue was triturated with IPE to give N¹-(cyclohexylmethyl)-N²-{3-fluoro-5-[(1E) -3-(hydroxyamino)-3-oxoprop-1-en-1-yl]pyridin-2-yl}-D-valinamide (120 mg).

TABLE 4
example number and chemical structure

| Ex | Str. |
|---|---|
| 1 | 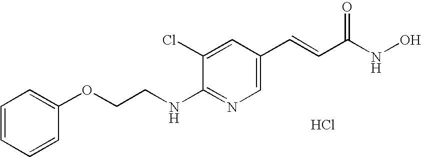 |
| 2 | 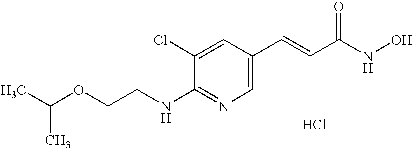 |
| 3 | 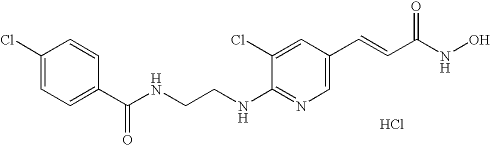 |
| 4 | 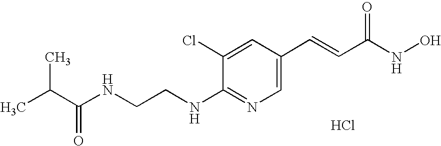 |
| 5 | 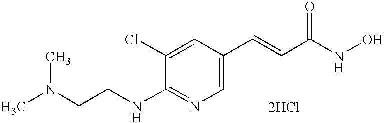 |
| 6 | 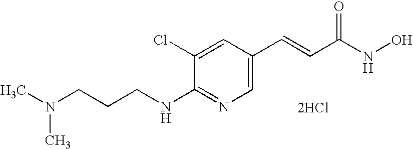 |
| 7 | 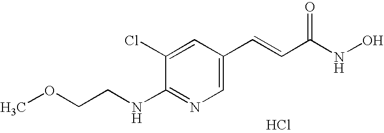 |
| 8 | 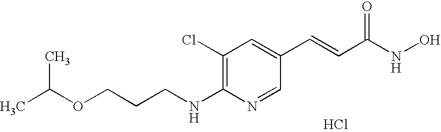 |
| 9 | 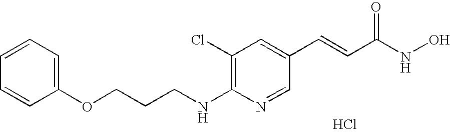 |
| 10 | 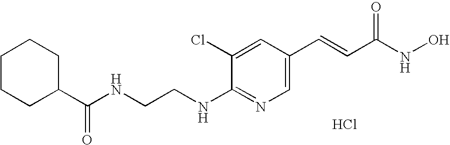 |
| 11 | 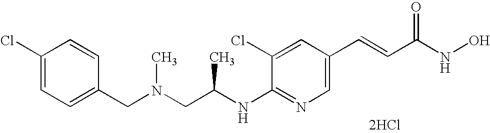 |
| 12 | 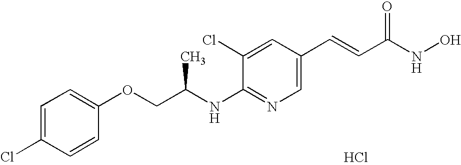 |
| 13 | 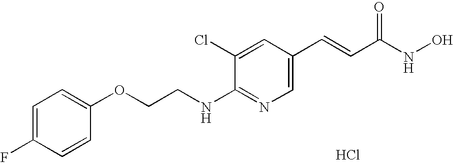 |
| 14 | 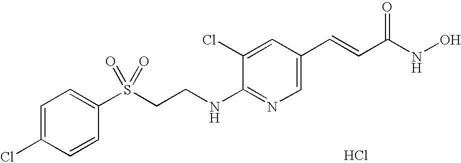 |
| 15 | 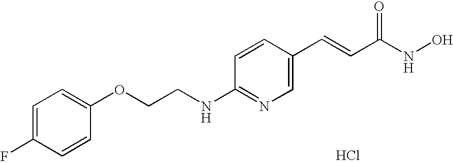 |
| 16 | 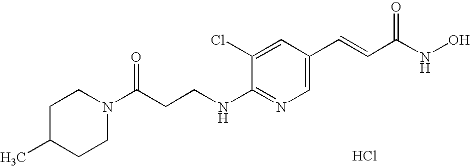 |
| 17 | 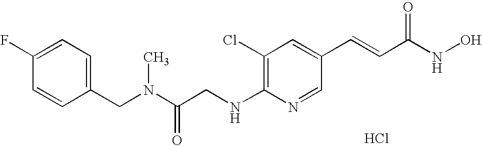 |
| 18 | 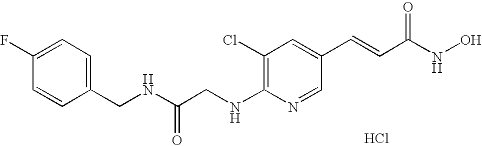 |
| 19 | 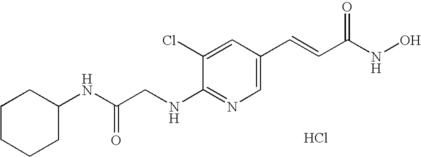 |
| 20 | 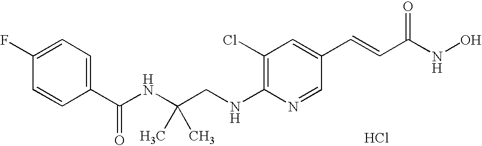 |
| 21 | 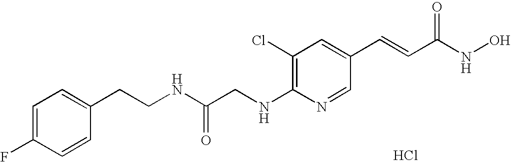 |
| 22 | 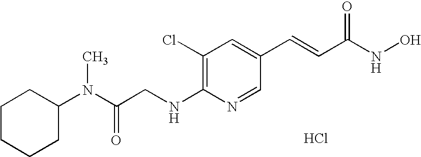 |
| 23 | 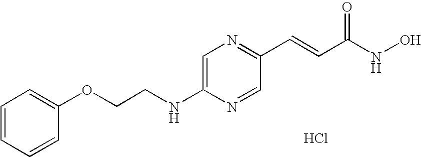 |
| 24 | 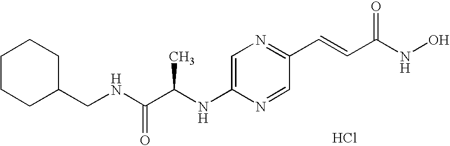 |
| 25 | 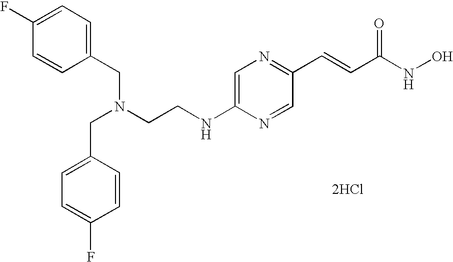 |
| 26 | 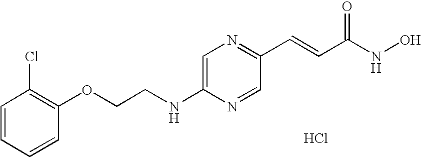 |
| 27 | 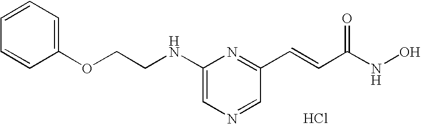 |
| 28 | 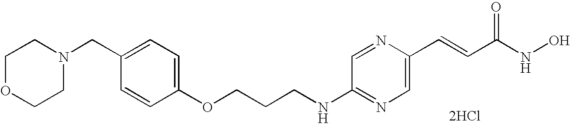 |
| 29 | 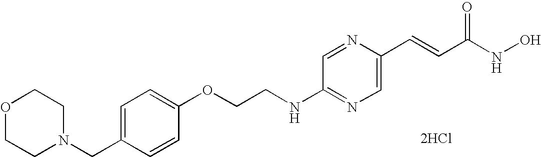 |
| 30 | 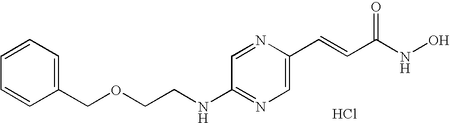 |
| 31 | 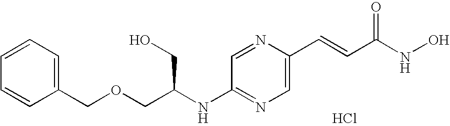 |
| 32 | 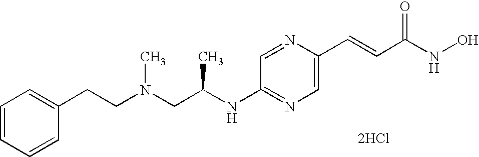 |
| 33 | 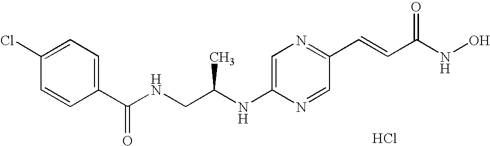 |
| 34 | 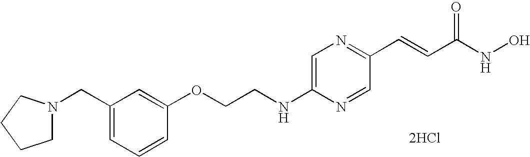 |
| 35 | 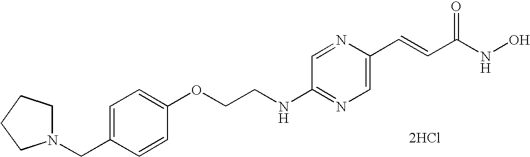 |
| 36 | 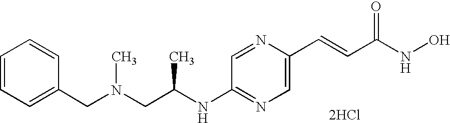 |
| 37 | 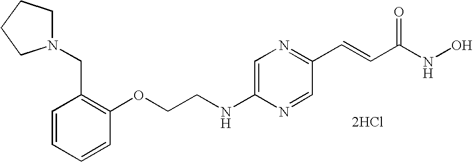 |
| 38 | 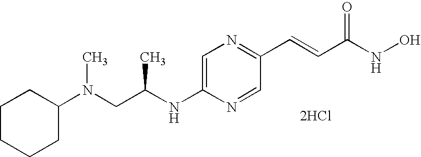 |
| 39 | 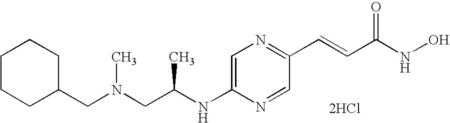 |
| 40 | 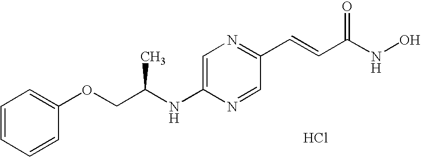 |
| 41 | 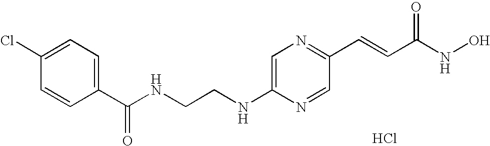 |
| 42 | 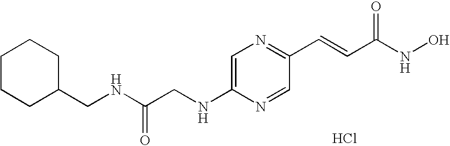 |
| 43 | 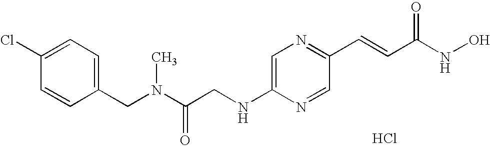 |
| 44 | 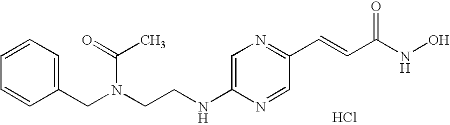 |
| 45 | 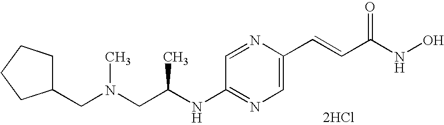 |
| 46 | 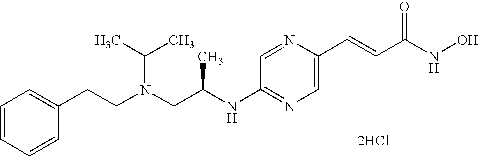 |
| 47 | 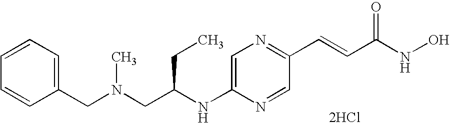 |
| 48 | 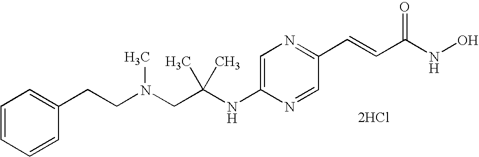 |
| 49 | 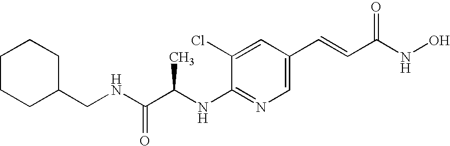 |
| 50 | 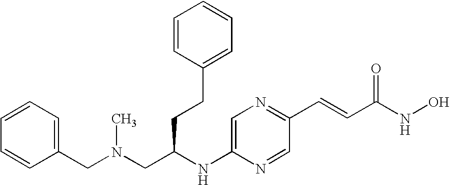 |
| 51 | 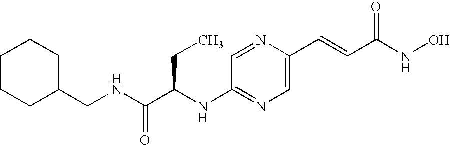 |
| 52 | 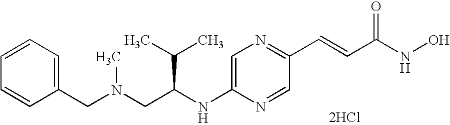 |
| 53 | 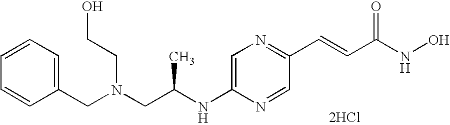 |
| 54 | 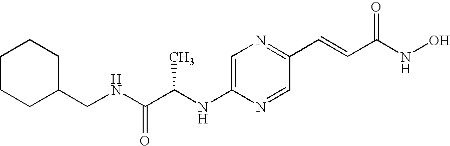 |
| 55 | 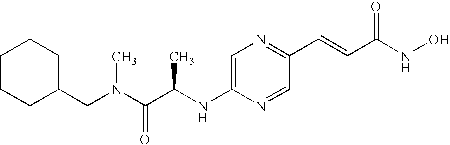 |
| 56 | 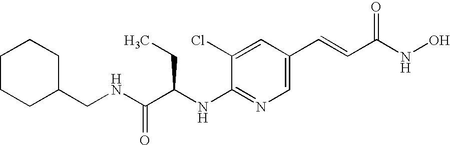 |
| 57 | 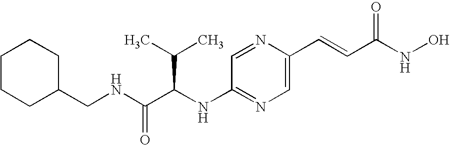 |
| 58 | 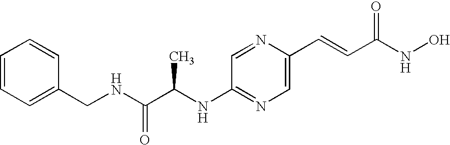 |
| 59 | 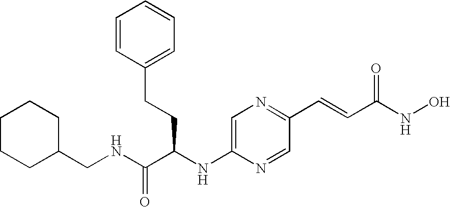 |
| 60 | 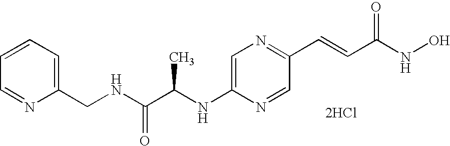 |
| 61 | 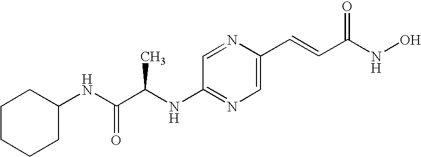 |
| 62 | 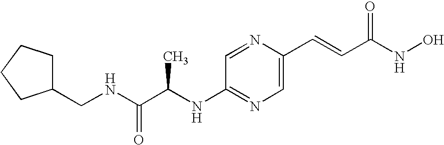 |
| 63 | 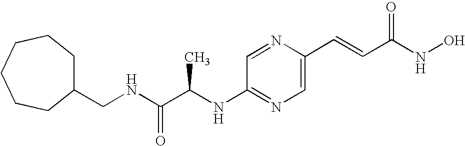 |
| 64 | 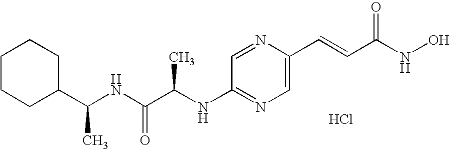 |
| 65 | 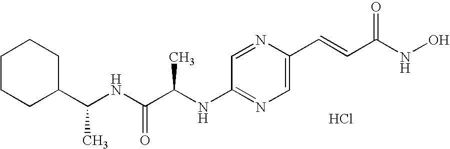 |
| 66 | 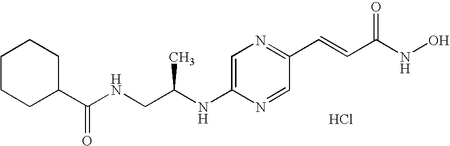 |
| 67 | 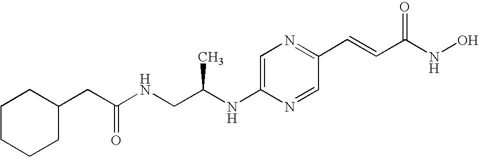 |
| 68 | 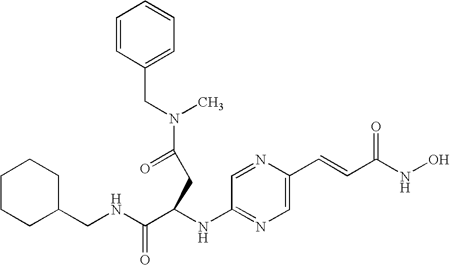 |
| 69 | 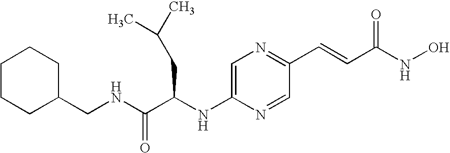 |
| 70 | 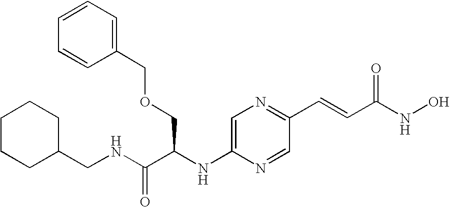 |
| 71 | 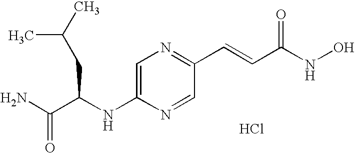 |
| 72 | 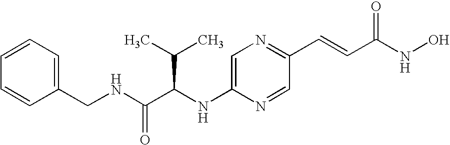 |
| 73 | 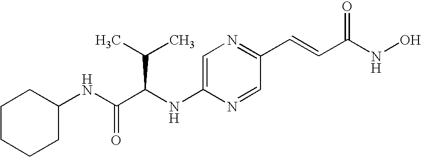 |
| 74 | 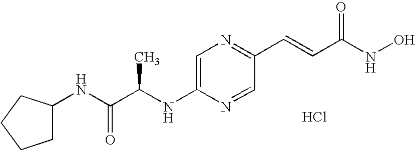 |
| 75 | 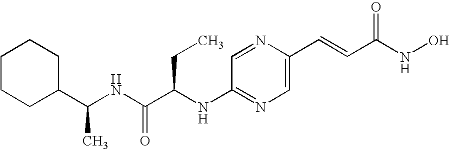 |
| 76 | 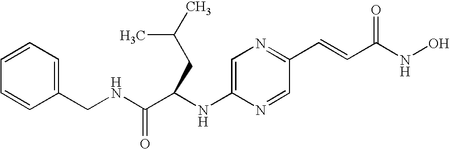 |
| 77 | 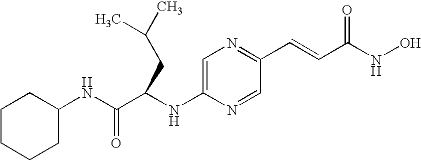 |
| 78 | 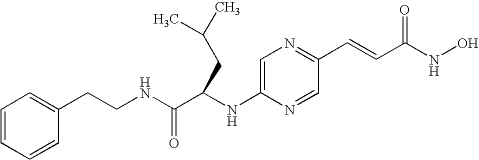 |
| 79 | 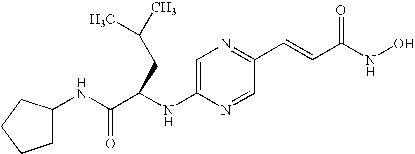 |
| 80 | 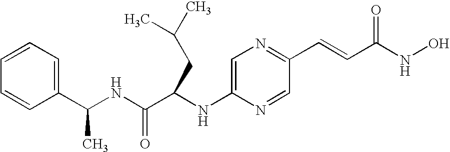 |
| 81 | 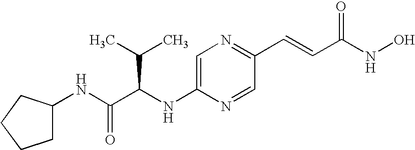 |
| 82 | 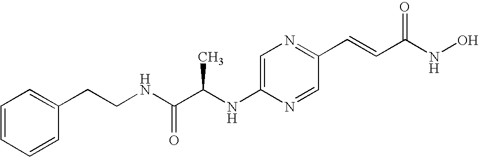 |
| 83 | 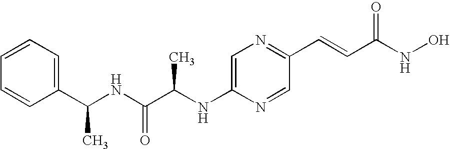 |
| 84 | 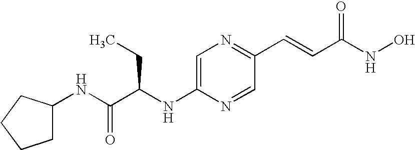 |
| 85 | 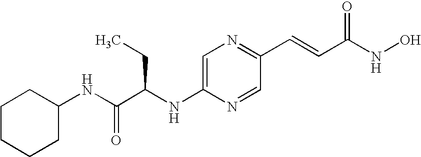 |
| 86 | 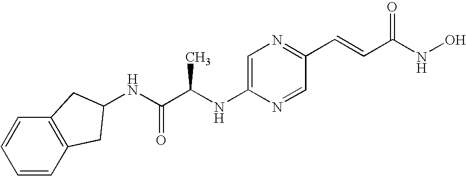 |
| 87 | 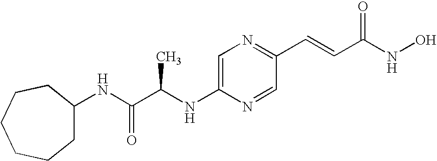 |
| 88 | 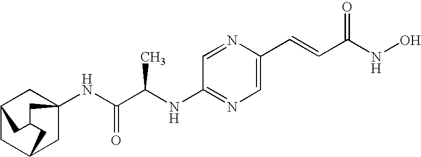 |
| 89 | 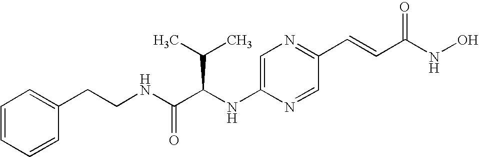 |
| 90 | 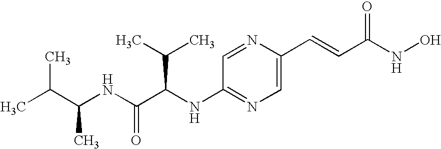 |
| 91 | 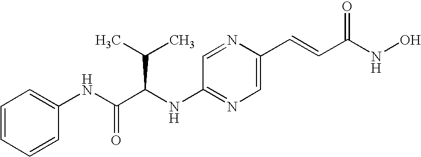 |
| 92 | 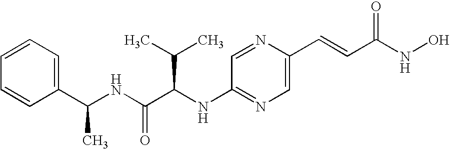 |
| 93 | 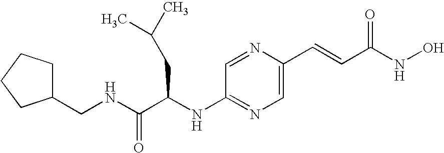 |
| 94 | 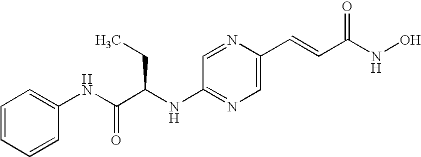 |
| 95 | 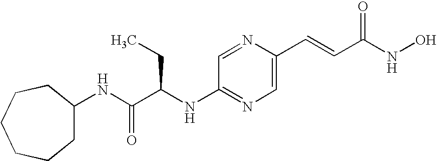 |
| 96 | 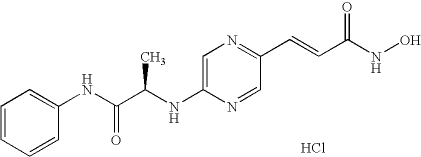 |
| 97 | 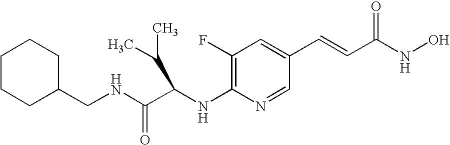 |

Ex: example number;
Str.: chemical structure;

TABLE 5
example number and analytical data

| Ex | Dat. |
|---|---|
| 1 | 1H-NMR (DMSO-d6): δ 3.86 (2H, t, J = 5.7 Hz), 4.16 (2H, t, |
| | J = 5.7 Hz), 6.42 (1H, d, J = 15.8 Hz), 6.87-7.00 (3H, m), |
| | 7.23-7.34 (2H, m), 7.38 (1H, d, J = 15.8 Hz), 8.03 (1H, s), |
| | 8.23 (1H, s), |
| | ESI-MS: 334 (M + H)+ |
| 2 | ESI-MS: 300 (M + H)+ |
| 3 | 1H-NMR (DMSO-d6): δ 3.43-3.77 (4H, m), 6.41 (1H, d, |
| | J = 15.9 Hz), 7.38 (1H, d, J = 15.9 Hz), 7.54 (2H, d, J = 8.5 Hz), |
| | 7.89 (2H, d, J = 8.5 Hz), 8.03 (1H, s), 8.20 (1H, s), 8.82 (1H, |
| | t, J = 5.1 Hz) |
| | ESI-MS: 395 and 396 (M + H)+ |
| 4 | ESI-MS: 327 (M + H)+ |
| 5 | ESI-MS: 285 (M + H)+ |
| 6 | ESI-MS: 299 (M + H)+ |
| 7 | ESI-MS: 272 (M + H)+ |
| 8 | ESI-MS: 314 (M + H)+ |
| 9 | ESI-MS: 348 (M + H)+ |
| 10 | 1H-NMR (DMSO-d6): δ 1.05-1.42 (5H, m), 1.52-1.72 (5H, m), |
| | 1.98-2.14 (1H, m), 3.20-3.34 (2H, m), 3.40-3.55 (2H, m), |
| | 6.34 (1H, d, J = 15.8 Hz), 7.35 (1H, d, J = 15.8 Hz), 7.89 (1H, |
| | t, J = 5.4 Hz), 7.95 (1H, s), 8.18 (1H, s) |
| | ESI-MS: 367 (M + H)+ |
| 11 | 1H-NMR (DMSO-d6): δ 1.15-1.34 (3H, m), 2.70-2.84 (3H, m), |
| | 3.03-3.54 (2H, m), 4.27-4.41 (2H, m), 4.61-4.83 (1H, m), |
| | 6.38 (1H, d, J = 15.7 Hz), 6.93 (1H, d, J = 8.3 Hz), |
| | 7.26-7.70 (4H, m), 7.35 (1H, d, J = 15.7 Hz), 7.88 (1H, s), |
| | 8.17 (1H, s) |
| | ESI-MS: 409 and 411 (M + H)+ |
| 12 | ESI-MS: 382 and 384 (M + H)+ |
| 13 | ESI-MS: 352 (M + H)+ |
| 14 | ESI-MS: 416 and 418 (M + H)+ |
| 15 | 1H-NMR (DMSO-d6): δ 3.76-3.92 (2H, m), 4.18 (2H, t, J = 4.9 Hz), |
| | 6.44 (1H, d, J = 15.8 Hz), 6.90-7.04 (2H, m), |
| | 7.06-7.23 (3H, m), 7.45 (1H, d, J = 15.8 Hz), 8.08 (1H, d, |
| | J = 9.2 Hz), 8.19 (1H, s), 9.34 (1H, br-s) |
| | ESI-MS: 318 (M + H)+ |
| 16 | ESI-MS: 367 (M + H)+ |
| 17 | ESI-MS: 393 (M + H)+ |
| 18 | ESI-MS: 379 (M + H)+ |
| 19 | ESI-MS: 353 (M + H)+ |
| 20 | 1H-NMR (DMSO-d6): δ 1.42 (6H, s), 3.71 (2H, d, J = 4.9 Hz), |
| | 6.35 (1H, d, J = 15.8 Hz), 7.23-7.43 (3H, m), 7.47-7.60 (1H, |
| | m), 7.85 (2H, dd, J = 5.6 Hz, 8.8 Hz), 7.96 (1H, s), 8.21 (1H, |
| | s), 8.39 (1H, s) |
| | ESI-MS: 407 (M + H)+ |
| 21 | ESI-MS: 393 (M + H)+ |
| 22 | ESI-MS: 367 (M + H)+ |
| 23 | 1H-NMR (DMSO-d6): δ 3.73 (2H, t, J = 5.4 Hz), 4.14 (2H, t, |
| | J = 5.4 Hz), 6.64 (1H, d, J = 15.2 Hz), 6.91-6.98 (3H, m), |
| | 7.26-7.32 (2H, m), 7.40 (1H, d, J = 15.2 Hz), 8.13 (1H, s), |
| | 8.16 (1H, s) |
| | ESI-MS: 301 (M + H)+ |
| 24 | 1H-NMR (DMSO-d6): δ 0.75-0.89 (2H, m), 1.02-1.20 (3H, m), |
| | 1.28-1.42 (1H, m), 1.33 (3H, d, J = 7.0 Hz), 1.53-1.68 (5H, |
| | m), 2.83-2.95 (2H, m), 4.39 (1H, q, J = 7.0 Hz), 6.62 (1H, d, |
| | J = 15.2 Hz), 7.38 (1H, d, J = 15.2 Hz), 7.92 (1H, t, J = 5.9 Hz), |
| | 8.07 (1H, s), 8.12 (1H, s) |
| | ESI-MS: 348 (M + H)+ |
| 25 | ESI-MS: 440 (M + H)+ |
| 26 | 1H-NMR (DMSO-d6): δ 3.76 (2H, t, J = 5.5 Hz), 4.22 (2H, t, |
| | J = 5.5 Hz), 6.63 (1H, d, J = 15.3 Hz), 6.96 (1H, dt, |
| | J = 1.1 Hz, 7.8 Hz), 7.19 (1H, dd, J = 1.1 Hz, 8.3 Hz), |
| | 7.27-7.32 (1H, m), 7.39 (1H, d, J = 15.3 Hz), 7.41 (1H, dd, |
| | J = 1.6 Hz, 7.8 Hz), 8.13 (2H, s) |
| | ESI-MS: 335 (M + H)+ |
| 27 | ESI-MS: 301 (M + H)+ |
| 28 | ESI-MS: 414 (M + H)+ |
| 29 | ESI-MS: 400 (M + H)+ |
| 30 | ESI-MS: 315 (M + H)+ |
| 31 | ESI-MS: 345 (M + H)+ |
| 32 | 1H-NMR (DMSO-d6): δ 1.26 (3H, s), 2.81-3.53 (9H, m), |
| | 4.55 (1H, br-s), 6.64 (1H, d, J = 15.3 Hz), 7.22-7.45 (6H, m), |
| | 8.07 (1H, s), 8.12 (1H, s) |
| | ESI-MS: 356 (M + H)+ |
| 33 | 1H-NMR (DMSO-d6): δ 1.19 (3H, d, J = 7.0 Hz), 3.39-3.46 (2H, |
| | m), 4.18-4.29 (1H, m), 6.60 (1H, d, J = 15.2 Hz), 7.37 (1H, |
| | d, J = 15.2 Hz), 7.52 (2H, d, J = 8.6 Hz), 7.75-8.17 (1H, br), |
| | 7.86 (2H, d, J = 8.6 Hz), 8.05-8.09 (2H, m), 8.71 (1H, t, J = 5.8 Hz) |
| | ESI-MS: 376 (M + H)+, 398 (M + Na)+ |
| 34 | ESI-MS: 384 (M + H)+ |
| 35 | ESI-MS: 384 (M + H)+ |
| 36 | 1H-NMR (CD3OD): δ 1.31-1.39 (3H, m), 2.87 and 3.00 (total 3H, |
| | each s), 3.23-3.52 (2H, m), 4.31-4.77 (3H, m), 6.72 (1H, d, |
| | J = 15.2 Hz), 7.42-7.59 (6H, m), 8.13 and 8.19 (total 2H, each |
| | s) |
| | ESI-MS: 342 (M + H)+ |
| 37 | ESI-MS: 384 (M + H)+ |
| 38 | ESI-MS: 334 (M + H)+ |
| 39 | 1H-NMR (DMSO-d6): δ 0.60-1.32 (5H, m), 1.23 (3H, d, |
| | J = 6.4 Hz), 1.47-1.87 (6H, m), 2.70-3.44 (7H, m), 4.45-4.58 (1H, |
| | m), 6.65 (1H, d, J = 15.2 Hz), 7.40 (1H, d, J = 15.2 Hz), |
| | 8.03-8.19 (1H, m), 8.07 (1H, s), 8.14 (1H, s), 9.57 and |
| | 9.73 (total 1H, each s) |
| | ESI-MS: 348 (M + H)+ |
| 40 | ESI-MS: 315 (M + H)+ |
| 41 | ESI-MS: 362 (M + H)+ |
| 42 | ESI-MS: 334 (M + H)+ |
| 43 | ESI-MS: 376 (M + H)+ |
| 44 | ESI-MS: 356 (M + H)+ |
| 45 | ESI-MS: 334 (M + H)+ |
| 46 | 1H-NMR (DMSO-d6): δ 1.21-1.42 (9H, m), 2.98-3.44 (6H, m), |
| | 3.65-3.82 (1H, m), 4.43-4.60 (1H, m), 6.67 (1H, d, J = 15.3 Hz), |
| | 7.18-7.36 (5H, m), 7.41 (1H, d, J = 15.3 Hz), 8.11 (1H, |
| | s), 8.14 (1H, s), 8.18-8.29 (1H, m), 9.87 and 10.15 (total |
| | 1H, each s) |
| | ESI-MS: 384 (M + H)+ |
| 47 | ESI-MS: 356 (M + H)+ |
| 48 | ESI-MS: 370 (M + H)+ |
| 49 | 1H-NMR (DMSO-d6): δ 0.76-0.90 (2H, m), 1.04-1.21 (3H, m), |
| | 1.31-1.42 (1H, m), 1.37 (3H, d, J = 6.9 Hz), 1.54-1.69 (5H, |
| | m), 2.84-2.99 (2H, m), 4.48-4.57 (1H, m), 6.31 (1H, d, |
| | J = 15.7 Hz), 6.47-6.54 (1H, m), 7.34 (1H, d, J = 15.7 Hz), |
| | 7.89 (1H, s), 7.97 (1H, t, J = 5.8 Hz), 8.17 (1H, s), 10.62 (1H, |
| | s) |
| | ESI-MS: 381 (M + H)+ |
| 50 | 1H-NMR (DMSO-d6): δ 1.64-1.75 (1H, m), 1.97-2.09 (1H, m), |
| | 2.12 (3H, s), 2.42 (2H, d, J = 6.0 Hz), 2.53-2.71 (2H, m), |
| | 3.41 (1H, d, J = 13.0 Hz), 3.52 (1H, d, J = 13.0 Hz), |
| | 4.16-4.25 (1H, m), 6.58 (1H, d, J = 15.2 Hz), 7.13-7.30 (10H, |
| | m), 7.36 (1H, d, J = 15.2 Hz), 7.42 (1H, d, J = 8.2 Hz), |
| | 7.99 (1H, s), 8.06 (1H, s), 8.96 (1H, s), 10.70 (1H, s) |
| | ESI-MS: 432 (M + H)+ |
| 51 | 1H-NMR (DMSO-d6): δ 0.76-0.94 (2H, m), 0.91 (3H, t, J = 7.4 Hz), |
| | 1.05-1.20 (3H, m), 1.30-1.41 (1H, m), 1.54-1.80 (7H, |
| | m), 2.82-2.97 (2H, m), 4.26-4.33 (1H, m), 6.59 (1H, d, |
| | J = 15.2 Hz), 7.36 (1H, d, J = 15.2 Hz), 7.60 (1H, d, J = 7.5 Hz), |
| | 7.94 (1H, t, J = 5.8 Hz), 8.05 (1H, s), 8.10 (1H, s), 8.96 (1H, |
| | s), 10.70 (1H, s) |
| 51 | ESI-MS: 362 (M + H)+, 384 (M + Na)+ |
| 52 | ESI-MS: 370 (M + H)+ |
| 53 | ESI-MS: 372 (M + H)+ |
| 54 | ESI-MS: 348 (M + H)+ |
| 55 | ESI-MS: 362 (M + H)+ |
| 56 | ESI-MS: 417 and 419 (M + Na)+ |
| 57 | 1H-NMR (DMSO-d6): δ 0.76-0.89 (2H, m), 0.91 (3H, d, |
| | J = 6.7 Hz), 0.92 (3H, d, J = 6.7 Hz), 1.05-1.19 (3H, m), |
| | 1.30-1.42 (1H, m), 1.54-1.68 (5H, m), 2.02-2.12 (1H, m), |
| | 2.81-2.98 (2H, m), 4.28-4.34 (1H, m), 6.59 (1H, d, J = 15.2 Hz), |
| | 7.36 (1H, d, J = 15.2 Hz), 7.52 (1H, d, J = 8.4 Hz), |
| | 7.96 (1H, t, J = 5.8 Hz), 8.04 (1H, s), 8.16 (1H, s), 8.95 (1H, |
| | s), 10.70 (1H, s) |
| | ESI-MS: 376 (M + H)+, 398 (M + Na)+ |
| 58 | ESI-MS: 364 (M + Na)+, 705 (2M + Na)+ |
| 59 | ESI-MS: 460 (M + Na)+ |
| 60 | ESI-MS: 341 (M − H)−, 365 (M + Na)+ |
| 61 | ESI-MS: 334 (M + H)+, 356 (M + Na)+ |
| 62 | ESI-MS: 334 (M + H)+, 356 (M + Na)+ |
| 63 | ESI-MS: 384 (M + Na)+ |
| 64 | 1H-NMR (DMSO-d6): δ 0.79-1.36 (6H, m), 0.96 (3H, d, |
| | J = 6.7 Hz), 1.32 (3H, d, J = 7.0 Hz), 1.54-1.74 (5H, m), |
| | 3.53-3.63 (1H, m), 4.40 (1H, q, J = 7.0 Hz), 6.63 (1H, d, |
| | J = 15.2 Hz), 7.38 (1H, d, J = 15.2 Hz), 7.57-8.44 (2H, br), |
| | 7.77 (1H, d, J = 8.8 Hz), 8.06 (1H, s), 8.14 (1H, s) |
| | ESI-MS: 384 (M + Na)+ |
| 65 | ESI-MS: 384 (M + Na)+ |
| 66 | 1H-NMR (DMSO-d6): δ 1.04-1.34 (5H, m), 1.11 (3H, d, |
| | J = 6.6 Hz), 1.54-1.71 (5H, m), 2.02-2.11 (1H, m), 3.14-3.22 (2H, |
| | m), 4.02-4.11 (1H, m), 6.60 (1H, d, J = 15.3 Hz), 7.38 (1H, |
| | d, J = 15.3 Hz), 7.82 (1H, t, J = 5.8 Hz), 8.05 (1H, s), |
| | 8.08 (1H, s) |
| | ESI-MS: 370 (M + Na)+ |
| 67 | 1H-NMR (DMSO-d6): δ 0.76-0.89 (2H, m), 1.00-1.20 (3H, m), |
| | 1.10 (3H, d, J = 6.6 Hz), 1.51-1.65 (6H, m), 1.92 (2H, d, |
| | J = 7.0 Hz), 3.13-3.21 (2H, m), 4.01-4.11 (1H, m), 6.57 (1H, d, |
| | J = 15.2 Hz), 7.30-7.36 (1H, m), 7.35 (1H, d, J = 15.2 Hz), |
| | 7.82 (1H, t, J = 5.8 Hz), 7.92 (1H, s), 8.07 (1H, s), 8.95 (1H, |
| | s), 10.68 (1H, s) |
| | ESI-MS: 384 (M + Na)+ |
| 68 | 1H-NMR (DMSO-d6): δ 0.72-0.87 (2H, m), 1.00-1.18 (3H, m), |
| | 1.29-1.42 (1H, m), 1.51-1.68 (5H, m), 2.75-2.96 (7H, m), |
| | 4.42-4.66 (2H, m), 4.80-4.91 (1H, m), 6.60 and 6.61 (total |
| | 1H, each d, J = each 15.2 Hz), 7.15-7.42 (6H, m), 6.67 and |
| | 7.72 (total 1H, each d, J = each 7.9 Hz), 7.87 and 7.93 (total |
| | 1H, each t, J = each 5.9 Hz), 8.05-8.13 (2H, m), 8.97 (1H, |
| | s), 10.71 (1H, s) |
| | ESI-MS: 517 (M + Na)+ |
| 69 | ESI-MS: 390 (M + H)+ |
| 70 | ESI-MS: 454 (M + H)+ |
| 71 | ESI-MS: 294 (M + H)+ |
| 72 | ESI-MS: 392 (M + Na)+ |
| 73 | ESI-MS: 362 (M + H)+ |
| 74 | ESI-MS: 320 (M + H)+ |
| 75 | ESI-MS: 376 (M + H)+ |
| 76 | ESI-MS: 406 (M + Na)+ |
| 77 | ESI-MS: 374 (M − H)−, 398 (M + Na)+ |
| 78 | ESI-MS: 420 (M + Na)+ |
| 79 | ESI-MS: 384 (M + Na)+ |
| 80 | ESI-MS: 398 (M + H)+, 420 (M + Na)+ |
| 81 | 1H-NMR (DMSO-d6): δ 0.90 (3H, d, J = 6.8 Hz), 0.91 (3H, d, |
| | J = 6.7 Hz), 1.31-1.81 (8H, m), 1.98-2.08 (1H, m), |
| | 3.95-4.06 (1H, m), 4.30-4.37 (1H, m), 6.59 (1H, d, J = 15.2 Hz), |
| | 7.36 (1H, d, J = 15.2 Hz), 7.54 (1H, d, J = 8.7 Hz), |
| | 7.99 (1H, d, J = 7.0 Hz), 8.05 (1H, s), 8.16 (1H, s), 8.96 (1H, |
| | s), 10.70 (1H, s) |
| | ESI-MS: 348 (M + H)+, 3.70 (M + Na)+ |
| 82 | 1H-NMR (DMSO-d6): δ 1.28 (3H, d, J = 7.0 Hz), 2.69 (2H, t, |
| | J = 7.1 Hz), 3.20-3.35 (2H, m), 4.30-4.39 (1H, m), 6.61 (1H, |
| | d, J = 15.2 Hz), 7.13-7.21 (3H, m), 7.21-7.28 (2H, m), |
| | 7.38 (1H, d, J = 15.2 Hz), 7.64-7.23 (1H, m), 8.01 (1H, t, |
| | J = 5.6 Hz), 8.06 (1H, s), 8.08 (1H, s), 10.72 (1H, s) |
| | ESI-MS: 356 (M + H)+, 378 (M + Na)+ |
| 83 | 1H-NMR (DMSO-d6): δ 1.26-1.41 (6H, m), 4.40-4.51 (1H, m), |
| | 4.84-4.94 (1H, m), 6.59 (1H, d, J = 15.2 Hz), 7.16-7.23 (1H, |
| | m), 7.27-7.40 (5H, m), 7.68 (1H, d, J = 7.1 Hz), 8.04 (1H, s), |
| | 8.09 (1H, s), 7.46 (1H, d, J = 8.1 Hz), 9.01-10.70 (1H, br.s) |
| | ESI-MS: 356 (M + H)+, 378 (M + Na)+ |
| 84 | 1H-NMR (DMSO-d6): δ 0.89 (3H, t, J = 7.4 Hz), 1.30-1.82 (10H, |
| | m), 3.94-4.04 (1H, m), 4.29-4.37 (1H, m), 6.59 (1H, d, |
| | J = 15.3 Hz), 7.35 (1H, d, J = 15.3 Hz), 7.59 (1H, d, J = 7.8 Hz), |
| | 7.97 (1H, d, J = 7.3 Hz), 8.06 (1H, s), 8.10 (1H, s), 9.04 (1H, |
| | br.s), 10.65 (1H, br.s) |
| | ESI-MS: 334 (M + H)+, 356 (M + Na)+ |
| 85 | 1H-NMR (DMSO-d6): δ 0.89 (3H, t, J = 7.4 Hz), 1.04-1.31 (5H, |
| | m), 1.49-1.58 (1H, m), 1.59-1.78 (6H, m), 3.48-3.58 (1H, m), |
| | 4.29-4.37 (1H, m), 6.58 (1H, d, J = 15.2 Hz), 7.35 (1H, d, |
| | J = 15.2 Hz), 7.58 (1H, d, J = 7.9 Hz), 7.88 (1H, d, J = 7.9 Hz), |
| | 8.05 (1H, s), 8.10 (1H, s), 8.51-9.67 (1H, br), |
| | 9.67-11.40 (1H, br) |
| | ESI-MS: 348 (M + H)+, 370 (M + Na)+ |
| 86 | 1H-NMR (DMSO-d6): δ 1.31 (3H, d, J = 7.0 Hz), 2.70-2.81 (2H, |
| | m), 3.11-3.21 (2H, m), 4.34-4.50 (2H, m), 6.60 (1H, d, |
| | J = 15.2 Hz), 7.11-7.17 (2H, m), 7.17-7.24 (2H, m), 7.37 (1H, |
| | d, J = 15.2 Hz), 7.68 (1H, d, J = 7.2 Hz), 8.06 (2H, s), |
| | 8.29 (1H, d, J = 7.2 Hz), 8.97 (1H, s), 10.71 (1H, s) |
| | ESI-MS: 390 (M + Na)+ |
| 87 | 1H-NMR (DMSO-d6): δ 1.28 (3H, d, J = 7.0 Hz), 1.31-1.63 (10H, |
| | m), 1.65-1.77 (2H, m), 3.65-3.74 (1H, m), 4.33-4.42 (1H, m), |
| | 6.59 (1H, d, J = 15.2 Hz), 7.36 (1H, d, J = 15.2 Hz), 7.62 (1H, |
| | d, J = 7.3 Hz), 7.86 (1H, d, J = 8.0 Hz), 8.06 (2H, s), 8.97 (1H, |
| | s), 10.70 (1H, s) |
| | ESI-MS: 370 (M + Na)+, 346 (M − H)− |
| 88 | 1H-NMR (DMSO-d6): δ 1.27 (3H, d, J = 7.0 Hz), 1.54-1.65 (6H, |
| | m), 1.84-1.95 (6H, m), 1.96-2.03 (3H, m), 4.34-4.43 (1H, m), |
| | 6.59 (1H, d, J = 15.2 Hz), 7.36 (1H, d, J = 15.2 Hz), 7.42 (1H, |
| | s), 7.56 (1H, d, J = 7.5 Hz), 8.070 (1H, s), 8.075 (1H, s), |
| | 8.96 (1H, s), 10.70 (1H, s) |
| | ESI-MS: 408 (M + Na)+ |
| 89 | 1H-NMR (DMSO-d6): δ 0.87 (3H, dd, J = 1.3, 3.4 Hz), 1.04 (3H, |
| | d, J = 6 Hz), 1.99-2.09 (1H, m), 2.63-2.75 (2H, m), |
| | 3.22-3.39 (4H, m), 4.28 (1H, dd, J = 6.6, 8.3 Hz), 6.6 (1H, d, |
| | J = 15.2 Hz), 7.14-7.25 (5H, m), 7.38 (1H, d, J = 15.2 Hz), 7.53 (1H, |
| | d, J = 8.4 Hz), 8.05 (1H, s), 8.08 (1H, t, J = 5.6 Hz), |
| | 8.16 (1H, s), 8.97 (1H, br.s), 10.71 (1H, s) |
| | ESI-MS: 406 (M + Na)+ |
| 90 | 1H-NMR (DMSO-d6): δ 0.80 (6H, d, J = 6.8 Hz), 0.89-0.98 (9H, |
| | m), 1.54-1.63 (1H, m), 2.00-2.08 (1H, m), 3.54-3.64 (1H, m), |
| | 4.25-4.31 (1H, m), 6.58 (1H, d, J = 15.3 Hz), 7.36 (1H, d, |
| | J = 15.3 Hz), 7.56 (1H, d, J = 8.4 Hz), 7.76 (1H, d, J = 8.7 Hz), |
| | 8.04 (1H, s), 8.14 (1H, s), 8.96 (1H, br.s), 10.69 (1H, s) |
| | ESI-MS: 372 (M + Na)+ |
| 91 | 1H-NMR (DMSO-d6): δ 0.995 (3H, d, J = 6.6 Hz), 1.004 (3H, d, |
| | J = 6.6 Hz), 2.12-2.21 (1H, m), 4.48-4.55 (1H, m), 6.61 (1H, |
| | d, J = 15.2 Hz), 7.04 (1H, t, J = 7.3 Hz), 7.26-7.33 (2H, m), |
| | 7.37 (1H, d, J = 15.2 Hz), 7.61 (2H, d, J = 7.8 Hz), 7.74 (1H, |
| | d, J = 8.3 Hz), 8.08 (1H, s), 8.21 (1H, s), 8.97 (1H, s), |
| | 10.15 (1H, s), 10.71 (1H, s) |
| | ESI-MS: 378 (M + Na)+ |
| 92 | 1H-NMR (DMSO-d6): δ 0.96 (3H, d, J = 6.8 Hz), 1.34 (3H, d, |
| | J = 6.9 Hz), 2.04-2.14 (1H, m), 4.36 (1H, d, J = 7 Hz), |
| | 4.94 (1H, m), 6.61 (1H, d, J = 15.2 Hz), 7.16-7.36 (5H, m), |
| | 7.39 (1H, d, J = 15.2 Hz), 6.09 (1H, s), 8.18 (1H, s), 8.56 (1H, |
| | d, J = 8.2 Hz) |
| | ESI-MS: 384 (M + H)+, 406 (M + Na)+ |
| 93 | 1H-NMR (DMSO-d6): δ 0.83-0.94 (6H, m), 1.06-1.74 (11H, m), |
| | 1.90-2.50 (1H, m), 2.88-3.03 (3H, m), 4.37-4.44 (1H, m), |
| | 6.6 (1H, d, J = 15.2 Hz), 7.37 (1H, d, J = 15.2 Hz), 8.06 (1H, s), |
| | 8.09 (1H, s), 8.2 (1H, b.s) |
| | ESI-MS: 398 (M + Na)+ |
| 94 | 1H-NMR (DMSO-d6): δ 0.99 (3H, t, J = 7.4 Hz), 1.72-1.92 (2H, |
| | m), 4.46-4.54 (1H, m), 6.60 (1H, d, J = 15.2 Hz), |
| | 7.01-7.07 (1H, m), 7.26-7.33 (2H, m), 7.35 (1H, d, J = 15.2 Hz), |
| | 7.61 (2H, d, J = 7.5 Hz), 7.82 (1H, d, J = 7.4 Hz), |
| | 8.08 (1H, s), 8.15 (1H, s), 8.69-9.36 (1H, br.s), 10.13 (1H, |
| | s), 10.36-11.02 (1H, br.s) |
| | ESI-MS: 342 (M + H)+, 364 (M + Na)+ |
| 95 | 1H-NMR (DMSO-d6): δ 0.89 (3H, t, J = 7.4 Hz), 1.31-1.77 (14H, |
| | m), 3.68-3.77 (1H, m), 4.28-4.35 (1H, m), 6.58 (1H, d, |
| | J = 15.2 Hz), 7.36 (1H, d, J = 15.2 Hz), 7.59 (1H, d, J = 7.8 Hz), |
| | 7.93 (1H, d, J = 7.9 Hz), 8.06 (1H, s), 8.10 (1H, s), 8.97 (1H, |
| | s), 10.70 (1H, s) |
| | ESI-MS: 362 (M + H)+, 384 (M + Na)+ |
| 96 | 1H-NMR (DMSO-d6): δ 1.45 (3H, d, J = 7.0 Hz), 4.60 (1H, q, |
| | J = 7.0 Hz), 6.64 (1H, d, J = 15.2 Hz), 7.04 (1H, t, J = 7.4 Hz), |
| | 7.26-7.33 (2H, m), 7.37 (1H, d, J = 15.2 Hz), 7.55-8.30 (2H, |
| | br-s), 7.63 (2H, d, J = 7.5 Hz), 8.11 (1H, s), 8.16 (1H, s), |
| | 10.21 (1H, s) |
| | ESI-MS: 328 (M + H)+, 350 (M + Na)+ |
| 97 | 1H-NMR (DMSO-d6): δ 2.28 (3H, s), 2.95-3.98 (11H, m), |
| | 4.46-4.61 (1H, m), 6.64 (1H, dd, J = 15.2 and 2.4 Hz), |
| | 7.04-7.24 (4H, m), 7.40 (1H, d, J = 15.2 Hz), 8.06 (1H, d, |
| | J = 3.3 Hz), 8.15 (1H, d, J = 9.2 Hz), 8.22 (1H, br.s), |
| | 11.05-11.15 (1H, m) |
| | ESI-MS: 415.3 (M + Na)+ |

Ex: example number;
Dat.: analytical data;

1. A compound having the following formula (I): 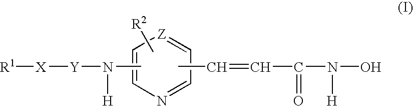
wherein
R¹ is hydrogen, optionally substituted lower alkyl, cyclo(lower)alkyl, cyclo(higher)alkyl, optionally substituted aryl, optionally substituted heterocyclyl, or aryl-fused cyclo(lower)alkyl,
R² is hydrogen or halogen,
Z is CH or N,
X is —O—, 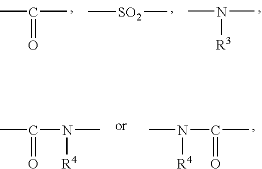
R³ is lower alkyl which may be substituted with —OH or optionally substituted aryl, or lower alkanoyl,
R⁴ is hydrogen or lower alkyl,
Y is optionally substituted lower alkylene,
or a pharmaceutically acceptable salt thereof.

2. The compound of claim 1, wherein a compound of the following formula (I′) 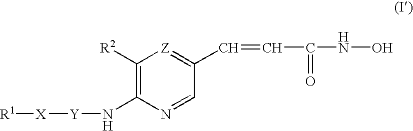
or a pharmaceutically acceptable salt thereof.

3. The compound of claim 2 wherein
R¹ is hydrogen, lower alkyl, cyclo(lower)alkyl(lower)alkyl, cyclo(higher)alkyl(lower)alkyl, optionally substituted ar(lower)alkyl, heteroaryl(lower)alkyl, cyclo(lower)alkyl, cyclo(higher)alkyl, optionally substituted aryl, lower alkyl heterocyclyl, aryl-fused cyclo(lower)alkyl,
R² is hydrogen or halogen,
Z is CH or N,
X is 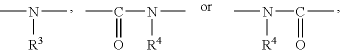
R³ is lower alkyl which may be substituted with —OH or aryl substituted with halogen, or lower alkanoyl,
R⁴ is hydrogen or lower alkyl,
Y is lower alkylene which may be substituted with hydroxy, aryl, aryl(lower)alkoxy, or carbamoyl optionally mono- or di-substituted with lower alkyl(s),
or a pharmaceutically acceptable salt thereof.

4. The compound of claim 3 wherein
R¹ is cyclo(lower)alkyl(lower)alkyl, ar(lower)alkyl which may be substituted with halogen, cyclo(lower)alkyl, cyclo(higher)alkyl, or aryl which may be substituted with halogen,
R² is hydrogen and Z is N, or R² is halogen and Z is CH,
X is 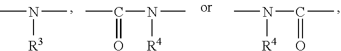
R³ is lower alkyl or lower alkanoyl,
R⁴ is hydrogen or lower alkyl,
Y is lower alkylene,
or a pharmaceutically acceptable salt thereof.

5. The compound of claim 4 wherein
R¹ is cyclohexylmethyl, benzyl, chlorobenzyl, cyclopentyl, cyclohexyl, cycloheptyl, adamantyl, phenyl or chlorophenyl,
R² is hydrogen and Z is N, or R² is fluorine or chlorine and Z is CH,
X is 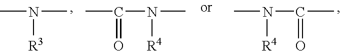
R³ is methyl or acetyl,
R⁴ is hydrogen or methyl,
Y is ethylene, methylmetylene, ethylmethylene, isopropylmethylene, propylene or isobutylmethylene,
or a pharmaceutically acceptable salt thereof.

6. A histone deacetylase inhibitor comprising the compound of claim 1.

7. A pharmaceutical composition for treating or preventing inflammatory disorders, diabetes, diabetic complications, homozygous thalassemia, fibrosis, cirrhosis, acute promyelocytic leukaemia (APL), organ transplant rejections, autoimmune diseases, protozoal infections or tumors, which comprises the compound of claim 1.

8. A pharmaceutical composition containing the compound of claim 1 as an active ingredient, in association with a pharmaceutically acceptable, substantially non-toxic carrier or excipient.

9. The compound of claim 1 for use as a medicament.

10. A method for inhibiting histone deacetylase, comprising using the compound of claim 1.

11. Use of the compound of claim 1 for the manufacture of a medicament for inhibiting histone deacetylase.

12. A method for treating or preventing inflammatory disorders, diabetes, diabetic complications, homozygous thalassemia, fibrosis, cirrhosis, acute promyelocytic leukaemia (APL), organ transplant rejections, autoimmune diseases, protozoal infections or tumors, which comprises administering an effective amount of the compound of claim 1 to a human being or an animal.

13. Use of the compound of claim 1 for the manufacture of a medicament for treating or preventing inflammatory disorders, diabetes, diabetic complications, homozygous thalassemia, fibrosis, cirrhosis, acute promyelocytic leukaemia (APL), organ transplant rejections, autoimmune diseases, protozoal infections or tumors.

14. A commercial package comprising the pharmaceutical composition of claim 1 and a written matter associated therewith, the written matter stating that the pharmaceutical composition may or should be used for treating or preventing inflammatory disorders, diabetes, diabetic complications, homozygous thalassemia, fibrosis, cirrhosis, acute promyelocytic leukaemia (APL), organ transplant rejections, autoimmune diseases, protozoal infections or tumors.
